(19) United States
(12) Patent Application Publication
(10) Pub. No.: US 2012/0034157 A1
(43) Pub. Date: Feb. 9, 2012

(54) Artificial cells

(75) Inventors: Roderick A. Hyde, Redmond, WA (US); Muriel Y. Ishikawa, Livermore, CA (US); Wayne A. Kindsvogel, Seattle, WA (US); Gary L. McKnight, Bothell, WA (US); Elizabeth A. Sweeney, Seattle, WA (US); Lowell L. Wood, JR., Bellevue, WA (US)

(73) Assignee: Searete LLC, a limited liability corporation of the State of Delaware

(21) Appl. No.: 12/806,067

(22) Filed: Aug. 3, 2010

(51) Int. Cl. A61K 51/04; A61K 9/127; C12N 5/07; A61K 49/00; A61K 9/14; A61P 37/04; A61K 39/00; C12N 5/10; B82Y 15/00

(52) U.S. Cl. 424/1.17; 424/184.1; 424/450; 435/325; 424/9.1; 424/1.21; 424/489; 977/773; 977/927

(57) ABSTRACT

The present disclosure relates to various embodiments associated with artificial cells, particularly artificial antigen presenting cells, methods of making the same, methods of administering the same, computer systems relating thereto, computer-implemented methods relating thereto, and associated computer program products.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC §119(e) for provisional patent applications, for any and all parent, grandparent, great-grandparent, etc. applications of the Related Application(s)). All subject matter of the Related Applications and of any and all parent, grandparent, great-grandparent, etc. applications of the Related Applications is incorporated herein by reference to the extent such subject matter is not inconsistent herewith.

### RELATED APPLICATIONS

For purposes of the USPTO extra-statutory requirements, the present application constitutes a continuation-in-part of United States Patent Application No. to be assigned, Docket No. 1004-002-014-000000, entitled ARTIFICIAL CELLS, naming Roderick A. Hyde, Muriel Y. Ishikawa, Wayne R. Kindsvogel, Gary L. McKnight, Elizabeth A. Sweeney and Lowell L. Wood, Jr. as inventors, filed 3 Aug. 2010, which is currently co-pending, or is an application of which a currently co-pending application is entitled to the benefit of the filing date.

For purposes of the USPTO extra-statutory requirements, the present application constitutes a continuation-in-part of United States Patent Application No. to be assigned, Docket No. 1004-002-014B-000000, entitled ARTIFICIAL CELLS, naming Roderick A. Hyde, Muriel Y. Ishikawa, Wayne R. Kindsvogel, Gary L. McKnight, Elizabeth A. Sweeney and Lowell L. Wood, Jr. as inventors, filed 3 Aug. 2010, which is currently co-pending, or is an application of which a currently co-pending application is entitled to the benefit of the filing date.

For purposes of the USPTO extra-statutory requirements, the present application constitutes a continuation-in-part of United States Patent Application No. to be assigned, Docket No. 1004-002-014C-000000, entitled ARTIFICIAL CELLS, naming Roderick A. Hyde, Muriel Y. Ishikawa, Wayne R. Kindsvogel, Gary L. McKnight, Elizabeth A. Sweeney and Lowell L. Wood, Jr. as inventors, filed 3 Aug. 2010, which is currently co-pending, or is an application of which a currently co-pending application is entitled to the benefit of the filing date.

For purposes of the USPTO extra-statutory requirements, the present application constitutes a continuation-in-part of United States Patent Application No. to be assigned, Docket No. 1004-002-014D-000000, entitled ARTIFICIAL CELLS, naming Roderick A. Hyde, Muriel Y. Ishikawa, Wayne R. Kindsvogel, Gary L. McKnight, Elizabeth A. Sweeney and Lowell L. Wood, Jr. as inventors, filed 3 Aug. 2010, which is currently co-pending, or is an application of which a currently co-pending application is entitled to the benefit of the filing date.

For purposes of the USPTO extra-statutory requirements, the present application constitutes a continuation-in-part of United States Patent Application No. to be assigned, Docket No. 1004-002-014E-000000, entitled ARTIFICIAL CELLS, naming Roderick A. Hyde, Muriel Y. Ishikawa, Wayne R. Kindsvogel, Gary L. McKnight, Elizabeth A. Sweeney and Lowell L. Wood, Jr. as inventors, filed 3 Aug. 2010, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/228,893, entitled BIOLOGICAL TARGETING COMPOSITIONS AND METHODS OF USING THE SAME, naming Roderick A. Hyde, Muriel Y. Ishikawa, Edward K. Y. Jung, William Gates, Alois A. Langer, Eric C. Leuthardt, Royce A. Levien, Clarence T. Tegreene, Thomas A. Weaver, Charles Whitmer, Lowell L. Wood, Jr. and Victoria Y. H. Wood as inventors, filed 13 Aug. 2008, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/228,892, entitled BIOLOGICAL TARGETING COMPOSITIONS AND METHODS OF USING THE SAME, naming Roderick A. Hyde, Muriel Y. Ishikawa, Edward K. Y. Jung, William Gates, Alois A. Langer, Eric C. Leuthardt, Royce A. Levien, Clarence T. Tegreene, Thomas A. Weaver, Charles Whitmer, Lowell L. Wood, Jr. and Victoria Y. H. Wood as inventors, filed 13 Aug. 2008, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/228,880, entitled BIOLOGICAL TARGETING COMPOSITIONS AND METHODS OF USING THE SAME, naming Roderick A. Hyde, Muriel Y. Ishikawa, Edward K. Y. Jung, William Gates, Alois A. Langer, Eric C. Leuthardt, Royce A. Levien, Clarence T. Tegreene, Thomas A. Weaver, Charles Whitmer, Lowell L. Wood, Jr. and Victoria Y. H. Wood as inventors, filed 13 Aug. 2008, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/228,869, entitled BIOLOGICAL TARGETING COMPOSITIONS AND METHODS OF USING THE SAME, naming Roderick A. Hyde, Muriel Y. Ishikawa, Edward K. Y. Jung, William Gates, Alois A. Langer, Eric C. Leuthardt, Royce A. Levien, Clarence T. Tegreene, Thomas. A. Weaver, Charles Whitmer, Lowell L. Wood, Jr. and Victoria Y. H. Wood as inventors, filed 13 Aug. 2008, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/228,868, entitled BIOLOGICAL TARGETING COMPOSITIONS AND METHODS OF USING THE SAME, naming Roderick A. Hyde, Muriel Y. Ishikawa, Edward K. Y. Jung, William Gates, Alois A. Langer, Eric C. Leuthardt, Royce A. Levien, Clarence T. Tegreene, Thomas A. Weaver, Charles Whitmer, Lowell L. Wood, Jr. and Victoria Y. H. Wood as inventors, filed 13 Aug. 2008, which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http.//www.uspto.gov////////.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary and/or admission as to whether or not the present application contains any new matter in addition to the matter of its parent application(s).

All subject matter of the Related Applications and of any and all parent, grandparent, great-grandparent, etc. applications of the Related Applications is incorporated herein by reference to the extent such subject matter is not inconsistent herewith.

### SUMMARY

The present disclosure relates to artificial cells, devices for administering the same, as well as computer systems and computer-implemented methods for administering the same.

For example, in an embodiment, a composition includes a lipid surface including at least one artificial antigen presenting cell complex, the artificial antigen presenting cell complex including at least one epitope joined to at least one MHC receptor component, and at least one immunomodulatory molecule component joined to the at least one MHC receptor component.

In an embodiment, a composition includes a lipid surface including at least one actively controllable artificial antigen presenting cell complex, the at least one actively controllable antigen presenting cell complex including at least one epitope joined to at least one MHC receptor component.

In an embodiment, a composition includes a lipid surface including at least one actively controllable artificial antigen presenting cell complex, the at least one actively controllable antigen presenting cell complex including at least one epitope joined to at least one MHC receptor component.

In an embodiment, a composition includes a modified cell including at least one artificial antigen presenting cell complex, the at least one artificial antigen presenting cell complex including at least one epitope joined to at least one MEC receptor component; and at least one nanotube operably linked to a NKG2D receptor on the modified cell.

In an embodiment, a composition includes a polymeric vehicle including at least one artificial antigen presenting cell complex, the at least one artificial antigen presenting cell complex including at least one epitope joined to at least one MHC receptor component.

In an embodiment, a composition includes a lipid surface including at least one suite of artificial antigen presenting cell complexes, each suite including at least two artificial antigen presenting cell complexes, each artificial antigen presenting cell complex including at least one epitope joined to at least one MEC receptor component, wherein at least two artificial antigen presenting cell complexes include epitopes of different antigens.

In an embodiment, a composition includes a lipid surface including at least one -suite of artificial antigen presenting cell complexes, each suite including at least two artificial antigen presenting cell complexes, each artificial antigen presenting cell complex including at least one epitope joined to at least one MHC receptor component, wherein at least two artificial antigen presenting cell complexes include different epitopes of the same antigen.

In an embodiment, a composition includes a red blood cell including at least one artificial antigen presenting cell complex including at least one epitope joined to at least one MHC receptor.

In an embodiment, a composition includes a lipid surface including at least one artificial antigen presenting cell complex, the artificial antigen presenting cell complex including at least one epitope joined to at least one MHC receptor component, and at least one cell death-initiating component.

In an embodiment, a composition includes a lipid or polymeric vehicle (e.g., liposome, etc.) including at least one nanoparticle, and at least one antigen presenting cell complex including at least one epitope joined to at least one MHC receptor component.

Certain aspects include methods of making or administering a composition described herein.

Certain aspects relate to devices, computer systems, computer program products, and computer-implemented methods related to administering a composition described herein.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a schematic diagram illustrating the activation of a target-binding agent upon binding to a target molecule and exposure to light of a suitable wavelength and power.

FIG. 2 is a schematic diagram illustrating the interaction of a modified red blood cell with a target cell. The target-binding agent is activated upon binding to the target cell and singlet oxygen radical species is generated upon exposure to electromagnetic radiation of a suitable wavelength and power.

FIG. 3 is a schematic diagram illustrating the interaction of multiple target-binding agents with a target cell. The target-binding agents are activated upon binding to the target cell.

FIG. 4 is a schematic diagram illustrating the interaction of a population of modified red blood cells with a population of target cells. The target-binding agent is activated upon binding to the target cell.

FIG. 5 is a schematic diagram illustrating the interaction of a modified red blood cell with a target cell. Exposure of the activated target-binding agent to the electromagnetic radiation of a suitable wavelength and power produces a singlet oxygen radical molecule which, in turn, results in damage or death to the target cell.

FIG. 6 is a schematic diagram illustrating the interaction of a modified red blood cell with a target cell. The target-binding agent is activated upon binding of the target recognition moiety to the target molecule. Exposure of the activated target-binding agent to electromagnetic radiation of a suitable wavelength produces a singlet oxygen radical molecule which results in lysis of the modified red blood cell and release of one or more therapeutic agents.

FIG. 7 is a schematic diagram illustrating the interaction of a modified red blood cell with a target cell. The modified red blood cell comprises multiple target recognition moieties on its surface.

FIG. 8 is a schematic diagram illustrating the interaction of a modified red blood cell with a target cell. The target cell becomes internalized within the modified red blood cell, thereby activating the target recognition moiety of the target-binding agent.

FIG. 9 is a schematic diagram illustrating the interaction of a modified red blood with a target cell. The modified red blood cell becomes internalized into the target cell, where target molecules become bound to the target-binding agent, thereby activating the target recognition moiety of the target-binding agent.

FIG. 10 is a schematic diagram illustrating the interaction of a modified red blood cell with a target cell. The modified red blood cell becomes internalized into the target cell, where target molecules become bound to the target-binding agent, thereby activating the target recognition moiety of the target-binding agent. The activated target-binding agent produces a singlet oxygen radical molecule when it is exposed to electromagnetic radiation of a suitable wavelength and power, which results in lysis of the modified red blood cell and release of one or more therapeutic agents.

FIG. 11 is a schematic drawing illustrating particular aspects of an embodiment described herein.

FIG. 12 is a schematic drawing illustrating particular aspects of a vector in a cell or other vehicle.

FIG. 13 is a schematic drawing illustrating particular aspects of an inducible nucleic acid construct.

FIG. 14 illustrates a partial view of a particular embodiment of a device described herein.

FIG. 15 illustrates a partial view of a particular embodiment of a device described herein.

FIG. 16 illustrates a partial view of a particular embodiment of a device described herein.

FIG. 17 illustrates a partial view of a particular embodiment of a device described herein.

FIG. 18 illustrates a partial view of a particular embodiment of a device described herein.

FIG. 19 illustrates a partial view of a particular embodiment of a device described herein.

FIG. 20 illustrates a partial view of a particular embodiment of a device described herein.

FIG. 21 illustrates a partial view of a particular embodiment of a device described herein.

FIG. 22 illustrates a partial view of a particular embodiment of a device described herein.

FIG. 23 illustrates a partial view of a particular embodiment of a device described herein.

FIG. 24 illustrates a partial view of a particular embodiment of a device described herein.

FIG. 25 illustrates a partial view of a particular embodiment of a device described herein.

FIG. 26 illustrates a partial view of a particular embodiment of a system described herein.

FIG. 27 illustrates a partial view of a particular embodiment of a system described herein.

FIG. 28 illustrates a partial view of a particular embodiment of a system described herein.

FIG. 29 illustrates a partial view of a particular embodiment of a system described herein.

FIG. 30 illustrates a partial view of a particular embodiment of a system described herein.

FIG. 31 illustrates a partial view of a particular embodiment of a computer program product described herein.

FIG. 32 illustrates a partial view of a particular embodiment of a computer program product described herein.

FIG. 33 illustrates a partial view of a particular embodiment of a computer-implemented method described herein.

FIG. 34 illustrates a partial view of a particular embodiment of a computer-implemented method described herein.

FIG. 35 illustrates a partial view of a particular embodiment of a computer-implemented method described herein.

FIG. 36 illustrates a partial view of a particular embodiment of a computer-implemented method described herein.

FIG. 37 illustrates a partial view of a particular embodiment described in FIG. A, Prophetic Example 5, and a partial view of a particular embodiment described in FIG. A, Prophetic Example 7.

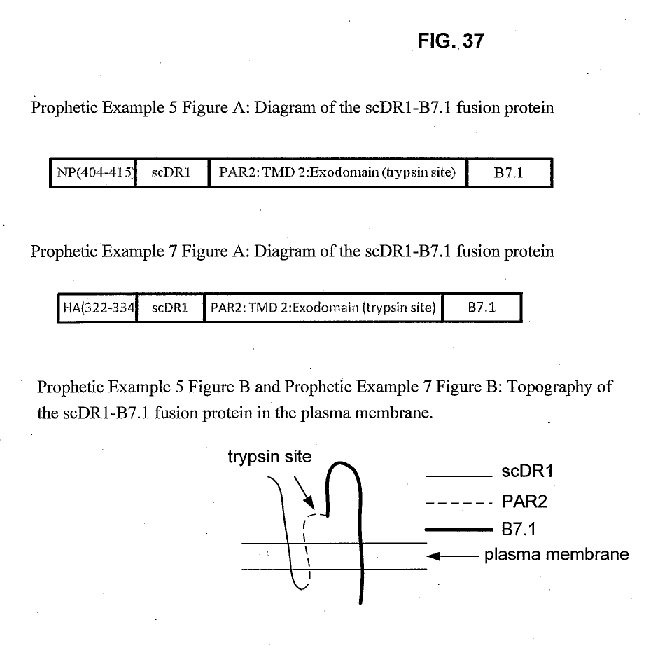

FIG. 38 illustrates a partial view of a particular embodiment described in FIG. A, Prophetic Example 6, a partial view of a particular embodiment described in FIG. B, Prophetic Example 6, and a partial view of a particular embodiment described in FIG. C, Prophetic Example 6.

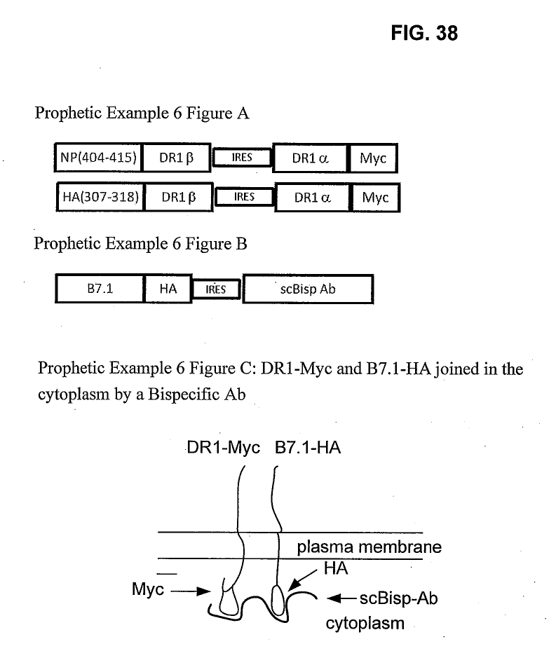

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The present disclosure relates to artificial antigen presenting cells, devices for administering the same, as well as computer systems and computer-implemented methods for administering the same. For example, artificial antigen presenting cells disclosed herein include major histocompatibility complex receptors that can be pre-loaded with a particular epitope, or epitopes, for administration as needed or as directed. In an embodiment, such epitopes relate to self-antigens, (e.g., in the case of autoimmune disease), or non-self antigens (e.g., in the case of organ or tissue transplantation, vaccination, allergy treatment, etc.). In an embodiment, the expression of the MHC plus epitope is configured to be passively or actively regulated.

Certain aspects described herein relate to artificial antigen presenting cells, which include lipid surfaces, polymeric vehicles, modified cells, or other vehicles bearing at least one artificial antigen presenting cell complex. In an embodiment, the at least one artificial antigen presenting cell complex includes at least one of an exogenous antigen presenting cell complex, synthetic antigen presenting cell complex, or endogenous antigen presenting cell complex that has been manipulated (for example, in vitro, ex vivo, in vivo, etc.). In an embodiment, the artificial antigen presenting cell complex includes at least one epitope joined to at least one MHC receptor component. In an embodiment, the artificial antigen presenting cell complex further includes at least one immunomodulatory molecule component joined to the at least one MHC receptor component.

In an embodiment, at least one of the epitope joined to the at least one MHC receptor component, or the at least one immunomodulatory molecule component joined to the at least one MHC receptor component is joined by at least one linker or linking component. In an embodiment, the linker includes, among other things, a cleavable linker (e.g., chemically cleavable linker, thermally cleavable linker, optically cleavable linker, enzymatically cleavable linker, etc.). In an embodiment, the linker includes at least one of an intracellular linker, extracellular linker, or a linker embedded in the lipid surface.

In an embodiment, at least one of the epitope joined to the at least one MHC receptor component, or the at least one immunomodulatory molecule component joined to the at least one MHC receptor component is joined by way of being a continuous molecule.

In an embodiment, at least one of the epitope joined to the at least one MHC receptor component, or the at least one immunomodulatory molecule component joined to the at least one MHC receptor component is joined by one or more of a fusion construct, antibody or portion thereof, chemical cross-linking, magnetic force, electrostatic force, hydrogen bond, hydrophobic force, van der Waals force, peptide bond, non-natural peptide bond, non-natural metallic bond, non-natural polymeric bond, or another physical or chemical means.

In an embodiment, two or more different immunomodulatory components are bound to the at least one MHC receptor component. In an embodiment, two or more different epitope-MHC receptor component combinations are bound to the at least one immunomodulatory component.

In an embodiment, at least a portion of the at least one artificial antigen presenting cell complex is bound, or anchored, to the lipid surface (e.g., by way of cholera toxin). See, for example, U.S. Patent App. Pub. No. 20040224009, which is incorporated herein by reference. In an embodiment, at least a portion of the at least one artificial antigen presenting cell complex is not bound to the lipid surface. In an embodiment, at least a portion of the at least one artificial antigen presenting cell complex is embedded in the lipid surface. In an embodiment, at least a portion of the at least one artificial antigen presenting cell complex transverses the lipid surface.

In an embodiment, the at least one immunomodulatory molecule component includes at least a portion of one or more of a co-stimulatory molecule, accessory molecule, adhesion molecule, cytokine, cytokine receptor, chemokine, chemokine receptor, anergy-inducing molecule, cell death-inducing molecule, or differentiation-inducing molecule.

In an embodiment, the lipid surface includes at least one of a two-dimensional or three-dimensional surface. In an embodiment, at least one of the at least one epitope joined to the at least one MHC receptor component or the at least one immunomodulatory molecule component joined to the at least one MHC receptor component of the at least one artificial antigen presenting cell complex is exposed to the interior of the three-dimensional surface. In an embodiment, at least one of the at least one epitope joined to the at least one MHC receptor component or the at least one immunomodulatory molecule component joined to the at least one MHC receptor component of the at least one artificial antigen presenting cell complex is exposed to the exterior of the three-dimensional surface. In an embodiment, both the at least one epitope joined to the at least one MHC receptor component and the at least one immunomodulatory molecule component joined to the at least one MHC receptor component of the at least one artificial antigen presenting cell complex are exposed to the interior of the three-dimensional surface. In an embodiment, both the at least one epitope joined to the at least one MHC receptor component and the at least one immunomodulatory molecule component joined to the at least one MHC receptor component of the at least one artificial antigen presenting cell complex are exposed to the exterior of the three-dimensional surface. That is, in an embodiment, the at least one epitope joined with the at least one MHC receptor component is directionally aligned with the at least one immunomodulatory component (if present), and directed toward either the interior or exterior of the vehicle (e.g., lipid surface, cell, polymeric vehicle, etc.). In an embodiment, the at least one epitope joined with the at least one MHC receptor component is not directionally aligned with the at least one immunomodulatory component (if present), and one component is directed toward the interior of the vehicle, while the other component is directed toward the exterior of the vehicle.

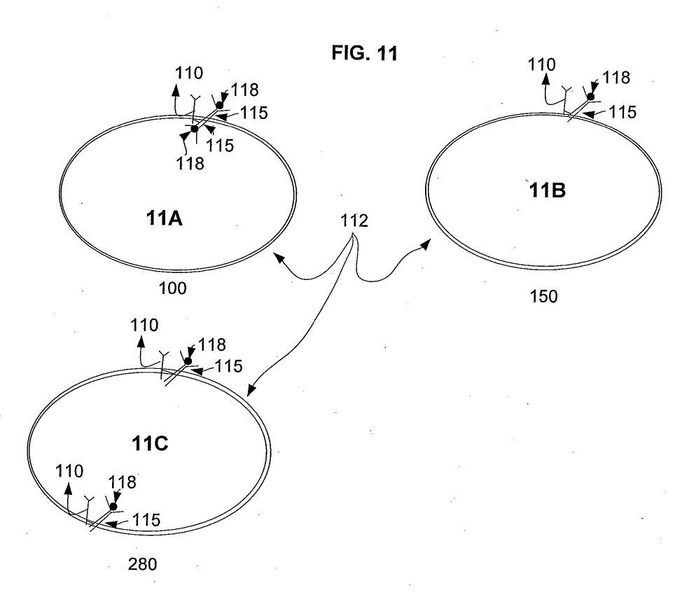

In an embodiment, the artificial antigen presenting cell complex is bi-directional or bi-functional, that is the artificial antigen presenting cell complex includes at least one first epitope joined with at least one first MHC receptor component (e.g., exposed to one side of the vehicle surface), and including at least one second epitope (which may be the same epitope or a different epitope as the first epitope, or which may be part of the same antigen or part of a different antigen from which the first epitope is derived) joined with at least one second MHC receptor component (e.g., exposed to the other side of the vehicle surface). See, for example, FIG. 11A.

For example, in an embodiment, the lipid surface includes at least a portion of at least one of a liposome, lipid droplet, chemical emulsion, phase separation, exosome, micelle, platelet, chip, device, cell, cerasome, lipid monolayer, lipid bilayer, or red blood cell ghost. In an embodiment, the cell includes at least one modified eukaryotic cell. In an embodiment the cell includes at least one of a bone cell, bone marrow cell, bone marrow stem cell, liver cell, liver stem cell, spleen cell, red blood cell, white blood cell, adipose cell, adipose stem cell, embryonic cell, embryonic stem cell, fetal cell, fetal stem cell, megakaryocyte, precursor cell, tumor cell, neuronal cell, mucosal cell, stomach cell, kidney cell, blood vessel cell, blood cell, skin cell, corneal cell, hair cell, antigen presenting cell, fungal cell, plant cell, egg cell, sperm cell, or other eukaryotic cell.

In an embodiment, the composition includes at least one nanotube operably linked to a NKG2D receptor on the cell. In an embodiment, the nanotube is sufficient for initiating cell-mediated death of the cell. In an embodiment, the nanotube is sufficient for cell-mediated release of intracellular contents of the cell. In an embodiment, release of intracellular contents of the cell is sufficient to initiate at least one immune response in a biological tissue or subject. In an embodiment, the at least one nanotube operably linked to a NKG2D receptor is actively controllable. In an embodiment, the at least one actively controllable nanotube is configured to be actively controlled by at least one of a change in pH, change in conductance, change in temperature, exposure to ultraviolet light, exposure to electromagnetic radiation, exposure to magnetic field, exposure to electrostatic charge, removal of magnetic field, removal of electrostatic charge, or exposure to at least one therapeutic agent.

In an embodiment, the cell further includes at least one cell death-initiating component. In an embodiment, the at least one cell death-initiating component includes at least one cell death-initiating nucleic acid construct. In an embodiment, the at least one cell death-initiating nucleic acid construct includes at least one inducible regulatory element. In an embodiment, the at least one cell death-initiating nucleic acid construct encodes at least one gene product sufficient to initiate death of the modified cell. In an embodiment, the at least one cell death-initiating nucleic acid construct is configured to initiate programmed cell death of the cell. In an embodiment, the at least one cell death-initiating nucleic acid construct is configured to initiate at least one of necrosis, pyroptosis, autophagocytosis, or apoptosis of the cella In an embodiment, the at least one cell death-initiating nucleic acid construct encodes at least one programmed cell death gene product.

In an embodiment, the at least one cell death-initiating nucleic acid construct encodes at least one of programmed cell death 1 gene (PDCD1), programmed cell death 2 gene (PDCD2), programmed cell death 3 gene (PDCD3), programmed cell death 4 gene (PDCD4), programmed cell death 5 gene (PDCD5), programmed cell death 6 gene (PDCD6), programmed cell death 7 gene (PDCD7), programmed cell death 8 gene (PDCD8), programmed cell death 9 gene (PDCD9), programmed cell death 10 gene (PDCD10), programmed cell death 11 gene (PDCD11), programmed cell death 12 gene (PDCD12), caspase gene, rel gene, hok gene, sok gene, diaminopimelate gene, nuclease gene, methylase gene, DNA ligase gene, DNA gyrase gene, toxin-antitoxin module, relF gene, triclosan, lysine, lysine-holin, Bcl-2-associated X protein (Bax), Bcl-2-associated death promoter (BAD), Bcl-2-homologous antagonist/killer (Bak), Bcl-2-related ovarian killer protein (Bok), Fas ligand, Fas receptor, or a foreign histocompatibility gene. In an embodiment, the toxin-antitoxin module includes at least one of masEF, chpBIK, relBE, yefM-yoeB, dinJ-yaf1, or ecnA-ecnB. In an embodiment, the at least one cell death-initating nucleic acid construct includes at least one gene product configured to lyse the at least one cell. In an embodiment, the at least one gene product to lyse the at least one cell includes at least one of a nuclease gene, or lysis gene E. In an embodiment, the at least one cell death-initating nucleic acid construct encodes at least one gene product configured to interfere with the utilization of at least one cellular metabolite.

In an embodiment, the at least one cell death-initiating component includes at least one receptor configured to allow entry of at least one of a toxin or pathogen. In an embodiment, the at least one cell death-initiating component includes at least one energy absorbing structure. In an embodiment, the energy absorbing structure includes at least one of a x-ray absorber, metal nanoparticle, or ultrasound absorber.

In an embodiment, the at least one artificial antigen presenting cell complex is actively controllable. For example, in an embodiment, the actively controllable artificial antigen presenting cell complex is configured to be actively controlled by at least one of a change in pH, change in conductance, change in temperature, exposure to ultraviolet light, exposure to electromagnetic radiation, exposure to magnetic field, exposure to electrostatic charge, removal of magnetic field, removal of electrostatic charge, or exposure to at least one therapeutic agent. In an embodiment, the actively controllable artificial antigen presenting cell complex includes at least one switchable complex (for example, by utilizing a switchable surface).

In an embodiment, a composition includes a polymeric vehicle including at least one artificial antigen presenting cell complex, the at least one artificial antigen presenting cell complex including at least one epitope joined to at least one MHC receptor component. In an embodiment, the at least one polymeric vehicle includes at least one of polyester, polylactic acid, polylactic-co-glycolic acid, cellulose, nitrocellulose, urea, urethane, phosphatidylcholine, cholesterol, phosphatidylethanolamine, hospholipid, ganglioside, dioleoylphosphatidylethanolamine, surfactant, or other polymer. In an embodiment, the at least one polymeric vehicle is at least one of biocompatible, biodegradable, or non-toxic.

In an embodiment, a composition includes a lipid surface including at least one suite of artificial antigen presenting cell complexes, each suite including at least two artificial antigen presenting cell complexes, each artificial antigen presenting cell complex including at least one epitope joined to at least one MHC receptor component, wherein at least two artificial antigen presenting cell complexes include epitopes of different antigens. In an embodiment, the epitopes include a different genetic variant. In an embodiment, at least one of the two or more antigen presenting cell complexes further includes at least one immunomodulatory molecule joined to the at least one MHC receptor component. In an embodiment, at least two of the two or more antigen presenting cell complexes include different immunomodulatory molecues joined to the at least one MHC receptor component. In an embodiment, at least two of the two or more antigen presenting cell complexes are different from each other. In an embodiment, a composition includes multiple lipid surfaces, each lipid surface including at least one epitope joined to at least one MHC receptor component, wherein at least two of the epitopes are different epitopes of the same antigen.

In an embodiment, a composition includes a lipid or polymeric vehicle (e.g., liposome, polymer, etc.) including at least one nanoparticle, and at least one antigen presenting cell complex including at least one epitope joined to at least one MHC receptor component. In an embodiment, the at least one nanoparticle includes at least one electronic identification device. In an embodiment, the at least one electronic identification device includes at least one radio frequency identification device (RFID).

Also described herein are modified red blood cells. More particularly, described herein include compositions comprising a red blood cell associated with a target recognition moiety and a fusion protein. In an embodiment, the modified red blood cell includes a photoactivatable molecule and a quencher molecule, wherein the target-binding agent emits at least one singlet oxygen radical molecule upon exposure to electromagnetic radiation (e.g., light) of a suitable wavelength when the target-binding agent is bound to a target molecule. Also described are targeted delivery of imaging agents, drugs, and peptide and protein pharmaceuticals using modified red blood cells. Processes for preparing the modified red blood cells, pharmaceutical and diagnostic compositions containing the same and methods of diagnosis and treatment involving the modified red blood cells are described. The specific compositions and methods described herein are intended as merely illustrative of their more general counterparts.

In this disclosure, many conventional techniques in molecular biology, protein biochemistry, cell biology, immunology, microbiology and recombinant DNA are described or referenced. These techniques are well-known and are explained in, e.g., Current Protocols in Mol. Biol., Vols. I-III, Ausubel, Ed. (1997); Sambrook et al., Mol. Cloning: A Lab. Manual, Second Ed. (Cold Spring Harbor Laboratory Press, Cold Spring Harbor, N.Y., 1989); DNA Cloning: A Practical Approach, Vols. I and II, Glover, Ed. (1985); Oligonuchotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Mol. Cloning; the series, Meth. Enzymol., (Academic Press, Inc., 1984); Gene Transfer Vectors for Mammalian Cells, Miller & Calos, Eds. (Cold Spring Harbor Laboratory, NY, 1987); and Meth. Enzymol., Vols. 154 and 155, Wu & Grossman, and Wu, Eds., respectively, and Strachan & Read, Human Mol. Genetics, Second Edition. (John Wiley and Sons, Inc., NY, 1999)).

As used in this specification and the appended claims, the singular forms “a,” “an” and “the” include plural referents unless the content clearly dictates otherwise. For example, reference to “a cell” include a single cell or may include a combination of two or more cells, and the like. Generally, the nomenclature used herein and the laboratory procedures in cell culture, molecular genetics, organic chemistry, analytical chemistry and nucleic acid chemistry and hybridization described below are those well known and commonly employed in the art.

Units, prefixes, and symbols may be denoted in their accepted SI form. Unless otherwise indicated, nucleic acids are written left to right in 5′ to 3′ orientation; amino acid sequences are written left to right in amino to carboxy orientation. Amino acids may be referred to herein by either their commonly known three letter symbols or by the one-letter symbols recommended by the IUPAC-IUBMB Nomenclature Commission. Nucleotides, likewise, may be referred to by their commonly accepted single-letter codes.

References cited herein are incorporated herein by reference to the extent not inconsistent with the instant disclosure and for all purposes to the same extent as if each individual publication, patent, or patent application was specifically and individually incorporated by reference.

In an embodiment, the composition including the at least one artificial antigen presenting cell is formulated for administration to at least one biological tissue by at least one route, including, among others, peroral, topical, transdermal, epidermal, intravenous, intraocular, tracheal, transmucosal, intracavity, subcutaneous, intramuscular, inhalation, fetal, intrauterine, intragastric, placental, intranasal, interdermal, intradermal, enteral, parenteral, surgical, or injection. In an embodiment, the intracavity route includes at least one of oral, vaginal, uterine, rectal, nasal, peritoneal, ventricular, or intestinal.

In an embodiment, the composition including the at least one antigen presenting cell is formulated for administration to at least one location in the at least one biological tissue and is translocatable to at least one other location in the at least one biological tissue.

In an embodiment, the composition includes one or more of a suspension, mixture, solution, sol, clathrate, colloid, emulsion, microemulsion, aerosol, ointment, capsule, micro-encapsule, powder, tablet, suppository, cream, device, paste, resin, liniment, lotion, ampule, elixir, spray, syrup, foam, pessary, tincture, detection material, polymer, biopolymer, buffer, adjuvant, diluent, lubricant, disintegration agent, suspending agent, solvent, light-emitting agent, colorimetric agent, glidant, anti-adherent, anti-static agent, surfactant, plasticizer, emulsifying agent, flavor, gum, sweetener, coating, binder, filler, compression aid, encapsulation aid, preservative, granulation agent, spheronization agent, stabilizer, adhesive, pigment, sorbent, nanoparticle, microparticle, prodrug, or gel.

In an embodiment, the composition further includes at least one nanoparticle. In an embodiment, the at least one nanoparticle includes at least one taggant, contrast agent, sensor, semiconductor, or electronic identification device. In an embodiment, the at least one electronic identification device includes at least one radio frequency identification device (RFID). In an embodiment, the at least one nanoparticle includes at least one of a diamagnetic particle, ferromagnetic particle, paramagnetic particle, super paramagnetic particle, particle with altered isotope, or other magnetic particle. In an embodiment, the at least one nanoparticle includes at least one quantum dot, silica nanoparticle, nanotube, or x-ray absorber. In an embodiment, the at least one nanoparticle includes at least one heavy metal. In an embodiment, the composition further comprises at least one radioactive, luminescent, colorimetric, or odorous substance. In an embodiment, the composition further comprises at least one photoactivatable molecule. In an embodiment, the at least one photoactivatable molecule includes psoralen. In an embodiment, the composition further comprises at least one quencher molecule. In an embodiment, the composition further comprises at least one target-binding agent.

As described herein, an antibody includes a polypeptide comprising a framework region from an immunoglobulin gene or fragments thereof that specifically binds and recognizes an antigen. Use of the term antibody is meant to include whole antibodies, including single-chain antibodies, antibody fragments, and antibody-related polypeptides. Antibody includes bispecific antibodies and multispecific antibodies so long as they exhibit the desired biological activity or function.

An antibody-related polypeptide includes antigen-binding antibody fragments, including single-chain antibodies that can comprise the variable region(s) alone, or in combination, with all or part of the following polypeptide elements: hinge region, CH₁, CH₂, and CH₃ domains of an antibody molecule. Also included are any combinations of variable region(s) and hinge region, CH₁, CH₂, and CH₃ domains. Antibody-related molecules useful as binding agents include, e.g., but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Examples include, but are not limited to: (i) a Fab fragment, a monovalent fragment consisting of the V_{L}, V_{H}, C_{L} and CH₁ domains; (ii) a F(ab′)₂ fragment, a bivalent fragment comprising two Fab fragments linked by a disulfide bridge at the hinge region; (iii) a Fd fragment consisting of the V_{H} and CH₁ domains; (iv) a Fv fragment consisting of the V_{L} and V_{H} domains of a single arm of an antibody, (v) a dAb fragment (Ward et al., Abstract, Nature 341: 544-546 (1989)), which consists of a V_{H} domain; and (vi) an isolated complementarity determining region (CDR). As such, antibody fragments may comprise a portion of a full length antibody, the antigen binding or variable region thereof. Examples of antibody fragments include, but are not limited to, Fab, Fab′, F(ab′)₂, and Fv fragments; diabodies; linear antibodies; single-chain antibody molecules; and multispecific antibodies formed from antibody fragments. Single-chain antibody molecules may comprise a polymer with a number of individual molecules, for example, dimmer, trimer or other polymers.

A biological sample includes sample material derived from or contacted by living cells. The term “biological sample” is intended to include tissues, cells and biological fluids isolated from a subject, as well as tissues, cells and fluids present within a subject. Biological samples include, e.g., but are not limited to, whole blood, plasma, semen, saliva, tears, urine, fecal material, sweat, buccal, skin, cerebrospinal fluid, and hair. Biological samples can also be obtained from biopsies of internal organs or from cancers. Biological samples can be obtained from subjects for diagnosis or research or can be obtained from undiseased individuals, as controls or for basic research.

A antineoplastic agent includes a chemical compound that can be used effectively to treat a neoplastic cell. An effective amount or pharmaceutically effective amount or therapeutically effective amount of a composition, includes a quantity of material sufficient to reasonably achieve a desired therapeutic and/or prophylactic effect. For example, it may include an amount that results in the prevention of, treatment of, or a decrease in, the symptoms associated with a disease or condition that is being treated, e.g., the diseases or medical conditions associated with a target polypeptide. The amount of a therapeutic composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds.

Electromagnetic radiation of a suitable wavelength includes one or more frequencies of electromagnetic radiation having one or more characteristics that taken as a whole are not considered unduly harmful to the subject. In illustrative non-limiting examples, such electromagnetic energy may include frequencies of optical light, optionally including visible light (detected by the human eye between approximately 400 nm and 700 nm) as well as infrared (longer than 700 nm) and limited spectral regions of ultraviolet light, such as UVA light (between approximately 320 nm and 400 nm). Electromagnetic energy includes, but is not limited to, single photon electromagnetic energy, two photon electromagnetic energy, multiple wavelength electromagnetic energy, and extended-spectrum electromagnetic energy.

An epitope includes any segment on an antigen to which an antibody or other ligand or binding molecule binds. An epitope may consist of chemically active surface groupings of molecules such as amino acids or sugar side chains and usually have specific three dimensional structural characteristics, as well as specific charge characteristics.

A monoclonal antibody includes an antibody obtained from a population of substantially homogeneous antibodies, i.e., the individual antibodies comprising the population are identical except for possible naturally occurring mutations that may be present in minor amounts. For example, a monoclonal antibody can be an antibody that is derived from a single clone, including any eukaryotic, prokaryotic, or phage clone, and not the method by which it is produced. A monoclonal antibody composition displays a single binding specificity and affinity for a particular epitope. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen.

A non-target tissue includes tissues of the subject which are not intended to be impaired or destroyed by the treatment method. These non-target tissues include but are not limited to healthy blood cells, and other normal tissue, not otherwise identified to be targeted.

A photoactivatable molecule or photosensitizing agent includes a chemical compound that upon exposure to photoactivating electromagnetic radiation is activated to release a singlet oxygen molecule. In an embodiment, the photoactivatable molecule itself, or some other species, is converted into a cytotoxic form, whereby target cells are killed or their proliferative potential diminished. Thus, photoactivatable molecule may exert their effects by a variety of mechanisms, directly or indirectly. For example, certain photoactivatable molecules become toxic when activated by light, for example by generating toxic species, e.g., oxidizing agents such as singlet oxygen or oxygen-derived free radicals, which are extremely destructive to cellular material and biomolecules such as lipids, proteins and nucleic acids. Porphyrins are of photosensitizing agents that act by generation of toxic oxygen species. Typically, the chemical compound is nontoxic to the animal to which it is administered or is capable of being formulated in a nontoxic composition, and the chemical compound in its photodegraded form is also nontoxic. A listing of representative photosensitive chemicals may be found in Kreimer-Bimbaurn, Sem. Hematol. 26:157-73 (1989).

A quencher, quencher molecule, or quenching molecule includes a moiety capable of preventing activation of the photoactivatable molecule when the target-binding agent is not bound to the target. Alternatively, the quencher may be capable of preventing the release of singlet oxygen from the target-binding agent when the target-binding agent is not bound to the target. In a suitable embodiment, the photoactivatable molecule is a porphyrin, and the quencher includes one or more suitable functional groups that coordinate to the axial position of the metal coordinated within the photoactivatable molecule. The target recognition moiety is positioned in the agent in such a way that the interaction of the target recognition moiety with the target disrupts the association of the axial ligand to the metal, releasing the quenching agent and allowing the porphyrin or porphyrin derivative tetrapyrrole to be activated when irradiated.

A subject includes, but is not limited to, a mammal, such as a human, but can also be an animal, e.g., domestic animals (e.g., dogs, cats and the like), farm animals (e.g., cows, sheep, pigs, horses and the like) and laboratory animals (e.g., monkey, rats, mice, rabbits, guinea pigs and the like). In an embodiment, a subject includes at least one of a bird, reptile, amphibian, fish, nonvertebrate, or plant.

A target includes the object that is intended to be detected, diagnosed, impaired or destroyed by the methods provided herein, and includes target cells, target tissues, and target compositions. Target cells are cells in target tissue, and the target tissue includes, but is not limited to, vascular endothelial tissue, abnormal vascular walls of tumors, solid tumors such as (but not limited to) tumors of the head and neck, tumors of the eye, tumors of the gastrointestinal tract, tumors of the liver, tumors of the breast, tumors of the prostate, tumors of the lung, nonsolid tumors and malignant cells of the hematopoietic and lymphoid tissue, neovascular tissue, other lesions in the vascular system, bone marrow, and tissue or cells related to autoimmune disease. Also included among target cells are cells undergoing substantially more rapid division as compared to non target cells, as well as pathogens such as bacteria, fungi, viruses, and parasites.

A target recognition moiety includes a molecule that is configured to specifically bind with a target. In an embodiment, the target recognition moiety is a member of a specific binding pair, e.g., an antigen; ligand; receptor; polyamide; peptide; carbohydrate; oligosaccharide; polysaccharide; low density lipoprotein (LDL) or an apoprotein of LDL; steroid; steroid derivative; hormone; hormone-mimic; lectin; drug; antibiotic; aptamer; DNA; RNA; lipid; or an antibody or antibody-related polypeptide. In an embodiment, the target recognition moiety includes at least one artificial antigen presenting cell complex.

In an embodiment, a therapeutic agent includes a compound or molecule that, when present in an effective amount, produces a desired therapeutic effect on a subject in need thereof. In an embodiment, a therapeutic agent includes at least a portion of one of an organic or inorganic small molecule, proteinoid, nucleic acid, peptide, polypeptide, protein, glycopeptide, glycolipid, lipoprotein, lipopolysaccharide, sphingolipid, glycosphingolipid, glycoprotein, peptidoglycan, lipid, carbohydrate, metalloprotein, proteoglycan, vitamin, mineral, amino acid, polymer, copolymer, monomer, prepolymer, cell receptor, adhesion molecule, cytokine, chemokine, immunoglobulin, antibody, antigen, extracellular matrix constituent, cell ligand, oligonucleotide, element, hormone, transcription factor, or contrast agent. In an embodiment, the at least one, therapeutic agent includes at least one converting enzyme responsive to the at least one prodrug or precursor compound. In an embodment the at least one enzyme includes at least one of glucuronidase or cytosine deaminase. In an embodiment, the at least one enzyme includes a nitrogen-reducing enzyme. In an embodiment, the nitrogen-reducing enzyme includes nitroreductase or a nitroreductase-like compound (e.g., an enzyme that uses FMN as a cofactor).

### I. Preparation of Modified Red Blood Cells

A. Preparation of Red Blood Cells

1. Isolation of Red Blood Cells

Mature red blood cells for use in generating the modified red blood cells may be isolated using various methods such as, for example, a cell washer, a continuous flow cell separator, density gradient separation, fluorescence-activated cell sorting (FACS), Miltenyi immunomagnetic depletion (MACS), or a combination of these methods (See, e.g., van den Berg et al., Clin. Chem. 33:1081-1082 (1987); Bar-Zvi et al., J. Biol. Chem. 262:17719-17723 (1987); Goodman et al., Abstract, Exp. Biol. Med. 232:1470-1476 (2007), each of which is incorporated herein by reference).

Red blood cells may be isolated from whole blood by simple centrifugation (See, e.g., van den Berg et al., Clin. Chem. 33:1081-1082 (1987)). For example, EDTA-anticoagulated whole blood may be centrifuged at 800×g for 10 min at 4° C. The platelet-rich plasma and buffy coat are removed and the red blood cells are washed three times with isotonic saline solution (NaCl, 9 g/L).

Alternatively, red blood cells may be isolated using density gradient centrifugation with various separation mediums such as, for example, Ficoll, Hypaque, Histopaque, Percoll, Sigmacell, or combinations thereof. For example, a volume of Histopaque-1077 is layered on top of an equal volume of Histopaque-1119. EDTA-anticoagulated whole blood diluted 1:1 in an equal volume of isotonic saline solution (NaCl, 9 g/L) is layered on top of the Histopaque and the sample is centrifuged at 700×g for 30 min at room temperature. Under these conditions, granulocytes migrate to the 1077/1119 interface, lymphocytes, other mononuclear cells and platelets remain at the plasma/1077 interface, and the red blood cells are pelleted. The red blood cells are washed twice with isotonic saline solution.

Alternatively, red blood cells may be isolated by centrifugation using a Percoll step gradient (See, e.g., Bar-Zvi et al., J. Biol. Chem. 262:17719-17723 (1987), which is incorporated herein by reference). As such, fresh blood is mixed with an anticoagulant solution containing 75 mM sodium citrate and 38 mM citric acid and the cells washed briefly in Hepes-buffered saline. Leukocytes and platelets are removed by adsorption with a mixture of α-cellulose and Sigmacell (1:1). The red blood cells are further isolated from reticulocytes and residual white blood cells by centrifugation through a 45/75% Percoll step gradient for 10 min at 2500 rpm in a Sorvall SS34 rotor. The red blood cells are recovered in the pellet while reticulocytes band at the 45/75% interface and the remaining white blood cells band at the 0/45% interface. The Percoll is removed from the red blood cells by several washes in Hepes-buffered saline. Other materials that may be used to generate density gradients for isolation of red blood cells include OptiPrep™, a 60% solution of iodixanol in water (from Axis-Shield, Dundee, Scotland).

Red blood cells may be separated from reticulocytes, for example, using flow cytometry (See, e.g., Goodman et al., Exp. Biol. Med. 232:1470-1476 (2007), which is incorporated herein by reference). In this instance, whole blood is centrifuged (550×g, 20 min, 25° C.) to separate cells from plasma. The cell pellet is resuspended in phosphate buffered saline solution and further fractionated on Ficoll-Paque (1.077 density), for example, by centrifugation (400×g, 30 min, 25° C.) to separate the red blood cells from the white blood cells. The resulting cell pellet is resuspended in RPMI supplemented with 10% fetal bovine serum and sorted on a FACS instrument such as, for example, a Becton Dickinson FACSCalibur (BD Biosciences, Franldin Lakes, N.J., USA) based on size and granularity.

Red blood cells may be isolated by immunomagnetic depletion (See, e.g., Goodman, et al., (2007) Exp. Biol. Med. 232:1470-1476, which is incorporated herein by reference). In this instance, magnetic beads with cell-type specific antibodies are used to eliminate non-red blood cells. For example, red blood cells are isolated from the majority of other blood components using a density gradient as described above followed by immunomagnetic depletion of any residual reticulocytes. The cells are pre-treated with human antibody serum for 20 min at 25° C. and then treated antibodies against reticulocyte specific antigens such as, for example, CD71 and CD36. The antibodies may be directly attached to magnetic beads or conjugated to PE, for example, to which magnetic beads with anti-PE antibody will react. As such, the antibody-magnetic bead complex is able to selectively extract residual reticulocytes, for example, from the red blood cell population.

Red blood cells may also be isolated using apheresis. The process of apheresis involves removal of whole blood from a patient or donor, separation of blood components using centrifugation or cell sorting, withdrawal of one or more of the separated portions, and transfusion of remaining components back into the patient or donor. A number of instruments are currently in use for this purpose such as for example the Amicus and Alyx instruments from Baxter (Deerfield, Ill., USA), the Trima Accel instrument from Gambro BCT (Lakewood, Colo., USA), and the MCS+9000 instrument from Haemonetics (Braintree, Mass., USA). Additional purification methods, such as those described above, may be necessary to achieve the appropriate degree of red blood cell purity.

2. Allogenic and Autologous Modified Red Blood Cells

In an embodiment, the modified red blood cells are autologous and/or allogeneic to the subject. In an embodiment, erythrocytes allogeneic to the subject include one or more of one or more blood type specific erythrocytes or one or more universal donor erythrocytes. In an embodiment, the modified red blood cells are fusion erythrocytes between erythrocytes autologous to the subject and one or more allogeneic erythrocytes, liposomes, and/or artificial vesicles.

For autologous transfusion, red blood cells, reticulocytes or hematopoietic stem cells from an individual are isolated and modified by methods described herein and retransfused into the individual.

For allogeneic transfusions, red blood cells, reticulocytes or hematopoietic stem cells are isolated from a donor, modified by methods described herein and transfused into another individual. In the instance where allogeneic cells are used for transfusion, care needs to be taken to use a compatible ABO blood group to prevent an acute intravascular hemolytic transfusion reaction. The latter is characterized by complement activation and lysis of incompatible red blood cells. The ABO blood types are defined based on the presence or absence of the blood type antigens A and B, monosaccharide carbohydrate structures that are found at the termini of oligosaccharide chains associated with glycoproteins and glycolipids on the surface of the red blood cells (reviewed in Liu et al., Abstract, Nat. Biotech. 25:454-464 (2007), which is incorporated herein by reference). Group O red blood cells lack either of these antigenic monosaccharide structures.

Individuals with group A red blood cells have naturally occurring antibodies to group B red blood cells whereas individuals with group B red blood cells have antibodies to group A red blood cells. Blood group AB individuals have neither antibody and blood group O individuals have both. Individuals with either anti-A and/or anti-B antibodies cannot receive a transfusion of blood containing the corresponding antigen. Because group O red blood cells contain neither A nor B antigens, they can be safely transfused into recipients of any ABO blood group, i.e., group A, B, AB, or O recipients. As such, group O red blood cells are considered “universal” and may be used in all blood transfusions. In contrast, group A red blood cells may be given to group A and AB recipients, group B red blood cells may be given to group B and AB recipients, and group AB red blood cells may only be given to AB recipients. As such, the modified red blood cells with an activatable molecular marker are matched for compatibility with the recipient.

In some instances, it may be beneficial to convert a non-group O modified red blood cell to a universal blood type. Enzymatic removal of the immunodominant monosaccharides on the surface of group A and group B red blood cells is one approach to generating a group O-like red blood cell population (See, e.g., Liu et al., Nat. Biotech. 25:454-464 (2007), which is incorporated herein by reference). Group B red blood cells may be converted using an α-galactosidase derived from green coffee beans, for example. Alternatively, α-N-acetylgalactosaminidase and α-galactosidase enzymatic activities derived from E. meningosepticum bacteria may be used to respectively remove the immunodominant A and B antigens (Liu et al., Nat. Biotech. 25:454-464 (2007), which is incorporated herein by reference). As such, packed red blood cells isolated as described above, are incubated in 200 mM glycine (pH 6.8) and 3 mM NaCl in the presence of either α-N-acetylgalactosaminidase and α-galactosidase (˜300 μg/ml packed red blood cells) for 60 min at 26° C. After treatment, the red blood cells are washed by 3-4 rinses in saline with centrifugation and ABO-typed according to standard blood banking techniques.

3. Derivation of Erythrocytes from Reticulocytes

In an embodiment, the red blood cells are differentiated ex vivo and/or in vivo from one or more reticulocytes. Modified reticulocytes may be used to generate mature red blood cells with monitoring and/or therapeutic properties. Reticulocytes are immature red blood cells and compose approximately 1% of the red blood cells in the human body. Reticulocytes develop and mature in the bone marrow. Once released into circulation, reticulocytes rapidly undergo terminal differentiation to mature red blood cells. Like mature red blood cells, reticulocytes do not have a cell nucleus. Unlike mature red blood cells, reticulocytes maintain the ability to perform protein synthesis. As such, the introduction of foreign messenger RNA (mRNA) into reticulocytes may facilitate synthesis and expression of exogenous proteins and/or peptides.

Reticulocytes of varying age may be isolated from peripheral blood based on the differences in cell density as the reticulocytes mature. As such, reticulocytes may be isolated from peripheral blood using differential centrifugation through various density gradients. For example, Percoll gradients may be used to isolate reticulocytes (See, e.g., Noble et al., Blood 74:475-481 (1989), which is incorporated herein by reference). Sterile isotonic Percoll solutions of density 1.096 and 1.058 g/ml are made by diluting Percoll (Sigma-Aldrich, Saint Louis, Mo., USA) to a final concentration of 10 mM triethanolamine, 117 mM NaCl, 5 mM glucose, and 1.5 mg/ml bovine serum albumin (BSA). These solutions have an osmolarity between 295 and 310 mOsm. Five milliliters, for example, of the first Percoll solution (density 1.096) is added to a sterile 15 ml conical centrifuge tube. Two milliliters, for example, of the second Percoll solution (density 1.058) is layered over the higher density first Percoll solution. Two to four milliliters of whole blood are layered on top of the tube. The tube is centrifuged at 250×g for 30 min in a refrigerated centrifuge with swing-out tube holders. Reticulocytes and some white cells migrate to the interface between the two Percoll layers. The cells at the interface are transferred to a new tube and washed twice with phosphate buffered saline (PBS) with 5 mM glucose, 0.03 mM sodium azide and 1 mg/ml BSA. Residual white blood cells are removed by chromatography in PBS over a size exclusion column.

Alternatively, reticulocytes may be isolated by positive selection using an immunomagnetic separation approach (See, e.g., Brun et al., Blood 76:2397-2403 (1990), which is incorporated herein by reference). This approach takes advantage of the large number of transferrin receptors that are expressed on the surface of reticulocytes relative to erythrocytes prior to maturation. As such, magnetic beads coated with an antibody to the transferrin receptor may be used to selectively isolate reticulocytes from a mixed red cell population. Antibodies to the transferrin receptor of a variety of mammalian species, including human, are available from commercial sources (e.g., Affinity BioReagents, Golden, Colo., USA; Sigma-Aldrich, Saint Louis, Mo., USA). The transferrin antibody may be directly linked to the magnetic beads. Alternatively, the transferrin antibody may be indirectly linked to the magnetic beads via a secondary antibody. For example, mouse monoclonal antibody 10D2 (Affinity BioReagents, Golden, Colo., USA) against human transferrin may be mixed with immunomagnetic beads coated with a sheep anti-mouse immunoglobulin G (Dynal/Invitrogen, Carlsbad, Calif., USA). The immunomagnetic beads are then incubated with a leukocyte-depleted red blood cell (RBC) fraction. The beads and RBCs are incubated at 22° C. with gentle mixing for 60-90 min followed by isolation of the beads with attached reticulocytes using a magnetic field. The isolated reticulocytes may be removed from the magnetic beads using, for example, DETACHaBEAD® solution (from Invitrogen, Carlsbad, Calif., USA). Alternatively, reticulocytes may be isolated from in vitro growth and maturation of CD34+ hematopoietic stem cells using the methods described below.

In general, the purity of the isolated reticulocytes may be assessed using microscopy in that reticulocytes are characterized by a reticular (mesh-like) network of ribosomal RNA that becomes visible under a microscope with certain stains such as new methylene blue or brilliant cresyl blue. Alternatively, analysis of creatine and hemoglobin A_{1C} content and pyruvate kinase, aspartate aminotransferase, and porphobilinogen deaminase enzyme activity may be used to assess properties of the isolated reticulocytes relative to mature erythrocytes (See, e.g., Brun et al., Blood 76:2397-2403 (1990), which is incorporated herein by reference). For example, the activity of porphobilinogen deaminase is nearly 9 fold higher whereas the hemoglobin A_{1C} content is nearly 10 fold less in reticulocytes relative to mature erythrocytes.

Modified reticulocytes may be transfused into an animal and allowed to differentiate into mature erythrocytes in vivo. Alternatively, modified reticulocytes may be differentiated into mature erythrocytes in vitro prior to transfusion. Maturation of reticulocytes in vitro may be carried out over several days using standard cell culture methods (See, e.g., Noble et al., Blood 74:475-481 (1998), which is incorporated herein by reference). For example, isolated reticulocytes are cultured for 3-5 days at 37° C. in Alpha-minimum essential medium (MEM) supplemented with 25 mM HEPES, 20 mg/dL glucose, 5% fetal bovine serum, 100 U/ml penicillin, and 0.1 mg/ml streptomycin, pH 7.5 at which time several assays may be done to assess maturation. For example, new methylene blue staining in combination with microscopy may be used to assess decline in the RNA-derived reticular network. Alternatively, the decline in the transferrin receptor expression as a function of maturation may be monitored using transferrin labeled, for example, with ¹²⁵I or FITC (Noble et al., Blood 74:475-481 (1998), which is incorporated herein by reference). In some instances, the analysis of creatine and hemoglobin A_{1C} content and pyruvate kinase, aspartate aminotransferase, and porphobilinogen deaminase enzyme activity may be used to assess maturation as described herein.

4. Differentiation of Red Blood Cells from Hematopoeitic Stem Cells

In an embodiment, the red blood cells are differentiated ex vivo and/or in vivo from one or more stem cells. In an embodiment, the one or more stem cells are one or more hematopoietic stem cells.

Red blood cells for use in generating one or more modified red blood cells may be derived from hematopoietic stem cells. Hematopoietic stem cells give rise to all of the blood cell types found in mammalian blood including myeloid (monocytes and macrophages, neutorphils, basophils, eosinophils, erythrocytes, megakaryocytes/platelets, dendritic cells) and lymphoid lineages (T-cells, B-cells, NK-cells). Hematopoietic stem cells may be isolated from the bone marrow of adult bones including, for example, femur, hip, rib, or sternum bones. Cells may be obtained directly from the hip, for example, by removal of cells from the bone marrow using aspiration with a needle and syringe. Alternatively, hematopoietic stem cells may be isolated from normal peripheral blood following pre-treatment with cytokines such as, for example, granulocyte colony stimulating factor (G-CSF). G-CSF mobilizes the release of cells from the bone marrow compartment into the peripheral circulation. Other sources of hematopoietic stem cells include umbilical cord blood and placenta.

Isolated hematopoietic stem cells may be cultured, expanded and differentiated ex vivo. For example, hematopoietic stem cells isolated from bone marrow, cytokine-stimulated peripheral blood or umbilical cord blood may be expanded and differentiated ex vivo into mature erythrocytes (Giarratana et al., Nature Biotech. 23:69-74 (2005); U.S. Patent Application 2007/0218552, each of which is incorporated herein by reference). As such, CD34+ cells are isolated from bone marrow or peripheral or cord blood using, for example, magnetic microbead selection and Mini-MACS columns (Miltenyi Biotech). The cells are subsequently cultured in modified serum-free medium supplemented with 1% bovine serum albumin (BSA), 120 μg/ml iron-saturated human transferrin, 900 ng/ml ferrous sulfate, 90 ng/ml ferric nitrate and 10 μg/ml insulin and maintained at 37° C. in 5% carbon dioxide in air. Expansion and differentiation of the cell culture may occur in multiple steps. For example, in the initial growth step following isolation, the cells may be expanded in the medium described herein in the presence of multiple growth factors including, for example, hydrocortisone, stem cell factor, IL-3, and erythropoietin. In the second stage, the cells may be co-cultured, for example, on an adherent stromal layer in the presence of erythropoietin. In a third stage, the cells may be cultured on an adherent stromal layer in culture medium in the absence of exogenous factors. The adherent stromal layer may be murine MS-5 stromal cells, for example. Alternatively, the adherent stromal layer may be mesenchymal stromal cells derived from adult bone marrow. The adherent stromal cells may be maintained in RPMI supplemented with 10% fetal calf serum, for example.

In some instances, it may be desirable to expand and partially differentiate the CD34+ hematopoietic stem cells in vitro and to allow terminal differentiation into mature erythrocytes to occur in vivo (See, e.g., Neildez-Nguyen et al., Nature Biotech. 20:467-472 (2002), which is incorporated herein by reference). As such, isolated CD34+ hematopoietic stem cells may be expanded in vitro in the absence of the adherent stromal cell layer in medium containing various factors including, for example, Flt3 ligand, stem cell factor, thrombopoietin, erythropoietin, and insulin growth factor. The resulting erythroid precursor cells, as judged by surface expression of CD36 and GPA, may be transfused into an animal where upon terminal differentiation to mature erythrocytes is allowed to occur.

Various assays may be performed to confirm the ex vivo differentiation of cultured hematopoietic stem cells into reticulocytes and erythrocytes, including, for example, microscopy, hematology, flow cytometry, deformability measurements, enzyme activities, and hemoglobin analysis and functional properties (Giarratana et al., Nature Biotech. 23:69-74 (2005), which is incorporated herein by reference). The phenotype of cultured hematopoietic stem cells may be assessed using microscopy of cells stained, for example, with Cresyl Brilliant blue. Reticulocytes, for example, exhibit a reticular network of ribosomal RNA under these staining conditions whereas erythrocytes are devoid of staining. Enucleated cells may also be monitored for standard hematological variables including mean corpuscular volume (MCV; fl), mean corpuscular hemoglobin concentration (MCHC; %) and mean corpuscular hemoglobin (MCH; pg/cell) using, for example, an XE2100 automat (Sysmex, Roche Diagnostics).

For the deformability measurements, for example, presumptive reticulocytes may be separated from nucleated cells on day 15 of culture, for example, by passage through a deleukocyting filter (e.g., Leucolab LCG2, Macopharma) and subsequently assayed using ektacytometry. As such, the enucleated cells are suspended in 4% polyvinylpyrrolidone solution and then exposed to an increasing osmotic gradient from 60 to 450 mosM, for example. Changes in the laser diffraction pattern (deformability index) of the cells are recorded as a function of osmolarity, to assess the dynamic deformability of the cell membrane. The maximum deformability index achieved at a physiologically relevant osmolarity is related to the mean surface area of red blood cells.

Alternatively, assays of hemoglobin may be used to assess the phenotype of differentiated cells (Giarratana et al., Nature Biotech. 23:69-74 (2005), which is incorporated herein by reference). For example, high performance liquid chromatography (HPLC) using a Bio-Rad Variant II Hb analyzer (Bio-Rad Laboratories) may be used to assess the percentage of various hemoglobin fractions. Oxygen equilibrium may be measured using a continuous method with a double-wavelength spectrophotometer (e.g., Hemox analyzer, TCS). The binding properties of hemoglobin may be assessed using flash photolysis. In this method, the rebinding of CO to intracellular hemoglobin tetramers are analyzed at 436 nm after photolysis with a 10 nanosecond pulse at 532 nm.

B. Target Recognition Moieties

The target-binding agents typically include one or more target recognition moieties for the selective binding of the composition to a target molecule. The target recognition moiety is configured to specifically bind to a target molecule of a particular cell, tissue, receptor, infecting agent or an area of the body of the subject to be treated, such as a target cell, target tissue or target composition.

Examples of target recognition moieties include, but are not limited to, an antigen; ligand; receptor; one member of a specific binding pair; polyamide; peptide; carbohydrate; oligosaccharide; polysaccharide; low density lipoprotein (LDL) or an apoprotein of LDL; steroid; steroid derivative; hormone; hormone-mimic; lectin; drug; antibiotic; aptamer; DNA; RNA; lipid; an antibody; an artificial antigen presenting cell complex, or an antibody-related polypeptide. In particular embodiments, the target recognition moiety is an antibody or antibody-related polypeptide. For example, antibodies useful as target recognition moieties include antibodies in general and monoclonal antibodies. The target recognition moiety can include a polypeptide having an affinity for a polysaccharide target, for example, a lectin (such as a seed, bean, root, bark, seaweed, fungal, bacterial, or invertebrate lectin). Particularly useful lectins include concanavalin A, which is obtained from jack beans, and lectins obtained from the lentil, Lens culinaris. The target recognition moiety can be a molecule or a macromolecular structure (e.g., a liposome, a micelle, a lipid vesicle, or the like) that preferentially associates or binds to a particular tissue, receptor, infecting agent or other area of the body of the subject to be treated.

Such targeting methods are contemplated herein for use in the instant target-binding agents. For non-limiting examples of targeting methods, See, e.g., U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542 and 5,709,874, each of which is incorporated herein by reference.

1. Antibodies as Target Recognition Moieties

Antibodies most ideal for use in subjects are those that are non-immunogenic when administered to the subject. Such antibodies have the advantages of exerting minimal side-effects, having long serum and biologic half-life, having wide bio-distribution, having high target specificity and high activity in engaging the effector phase of the immune system. These antibodies, when intended for human subjects, are commonly referred to as “humanized,” “human,” “chimeric,” or “primatized” antibodies; these are substantially (>70%) homologous to human amino acid sequences.

The target recognition moiety may be an antibody or an antigen binding antibody fragment configured to specifically bind to at least one epitope on the target molecule(s) associated with, produced by or on the surface of a target cell or tissue. The antibody or antibody fragment may be monospecific or multispecific. Both polyclonal and monoclonal antibodies may be used, as well as certain recombinant antibodies, such as chimeric and humanized antibodies and fusion proteins.

The target recognition moiety may be univalent, multivalent and/or multispecific. By “multivalent” it is meant that the target recognition moiety may bind more than one target, which may have the same or a different structure, simultaneously. By “multispecific” it is meant that the subject agents may bind to at least two targets which are of different structure. For example, a target recognition moiety having two different specificities would be considered multivalent and multispecific because it can bind two structurally different targets.

In some instances, the targeting antibody may be part of a multispecific antibody complex with one or more components that bind directly to a specific protein on the surface of the target cell (See, e.g., U.S. Pat. Nos. 5,470,570 and 5,843,440; U.S. Patent Applications 2003/0215454 A1 and 2006/0018912 A1, each of which is incorporated herein by reference). For example, the targeting antibody may be associated with a second antibody, such as a red blood cell binding antibody, that recognizes a protein on the surface of the red blood cell, e.g., α-N-acetylgalactosaminyltransferase, complement C4, aquaporin, complement decay-accelerating factor, band3 anion transport protein, Duffy antigen, glycophorin A, B and/or C, galactoside 2-L-fucosyltransferase 1, galactoside 2-L-fucosyltransferase 2, galactoside 3(4)-L-fusosyltransferase, CD44, Kell blood group glycoprotein, urea transporter, complement receptor protein (CR1), membrane transport protein XK, Landsteiner-Wiener blood group glycoprotein, Lutheran blood group glycoprotein, blood group RH (CE) polypeptide, blood group RH (D) polypeptide, Xg glycoprotein, acetylcholinesterase, anion exchanger, and/or insulin receptor (See, e.g., U.S. Patent Application 2006/0018912 A1, which is incorporated herein by reference). These multispecific antibodies are useful for the assembly of the modified red blood cells.

The antibodies within the multispecific antibody complex may be two or more intact antibodies and/or two or more antibody fragments such as, for example, Fab′, F(ab′)₂ and/or F, that are linked in some way to one another. The two or more antibodies may be fused by chemical conjugation, crosslinking and/or linker moieties. For example, polypeptides may be covalently bonded to one another through functional groups associated with the polypeptides such as, for example, carboxylic acid or free amine groups.

Alternatively, two or more antibodies may be linked through disulfide bonds. For example, the targeting antibody is reacted with N-succinimidyl S-acetylthioacetate (SATA) and subsequently deprotected by treatment with hydroxylamine to generate an SH-antibody with free sulfhydryl groups (See, e.g., U.S. Patent Application 2003/0215454 A1). The red blood cell binding antibody is reacted with sulfosuccinimidyl 4-(N-maelimidomethyl)cyclohexane-1-carboxylate (sSMCC). The two antibodies treated as such are purified by gel filtration and then reacted with one another to form a bispecific antibody complex.

Alternatively, the antibodies may be chemically cross-linked to form a heteropolymerized complex using, for example, SPDP [N-succinimidyl-3-(2-pyridyldithio)propionate] (See, e.g., Liu et al., Proc. Nat'l Acad. Sci. USA 82:8648-8652 (1985); U.S. Pat. No. 5,470,570, each of which is incorporated herein by reference). To generate the complex, the targeting antibody (1-2 mg/ml), for example, is incubated with a 7-fold molar excess of SPDP in phosphate buffered saline (PBS) for 45 minutes at room temperature. Excess SPDP is removed by dialysis overnight against two changes of PBS. Thiol groups are attached to the red blood cell binding antibody, for example, by incubating the antibody (1-3 mg/ml) with a 1000-fold molar excess of 2-iminothiolane in 12.5 mM sodium borate/PBS for 45 min at room temperature. Excess 2-iminothiolane is removed by dialysis as above. Equimolar amounts of the modified antibodies are incubated for 7 h at room temperature and the resulting heteropolymerized complex is separated from the uncoupled antibodies based on molecular weight using a standard sizing column.

Fab′ fragments from one or more antibodies may be generated, mixed together, and naturally occurring disulfide linkages reformed by oxidation. As such, a subset of the products will contain a Fab′ fragment from each antibody. Alternatively, Fab′ fragments from the targeting antibody, for example, may be activated with a bis-maleimide linker such as 1,1′-(methylenedi-4,1-phenylene)bis-maleimide and then linked to the Fab′ fragments from the red blood cell binding antibody through a disulfide bond (See, e.g., U.S. Patent Application 2003/0215454 A1, which is incorporated herein by reference).

Alternatively, the two antibody binding activities may be incorporated into a single fusion protein using recombinant DNA approaches (See, e.g., U.S. Pat. No. 6,132,992, which is incorporated herein by reference). For example, cDNA encoding the variable regions (V_{L} and V_{H}) of two antibodies directed against separate and distinct antigens, for example, may be combined into a linear expression construct from which a bispecific single-chain antibody may be produced (See, e.g., Haisma et al., Cancer Gene Ther. 7:901-904 (2000), which is incorporated herein by reference). As such, cDNA encoding the variable regions (V_{L} and V_{H}) of the targeting antibody and of the red blood cell binding antibody, for example, may be manipulated to form a bispecific single-chain antibody.

C. Photoactivatable Molecules

In an embodiment, the target-binding agents may include, but not be limited to, one or more photoactivatable molecules, such as a photosensitizer. Typically, the photoactivatable molecule becomes activated upon exposure to electromagnetic radiation. Various photoactivatable molecules are useful over the wavelength range of about 350 to about 1300 nm, the exact range being dependent upon the particular photosensitizer. In suitable embodiments, photoactivatable molecules are those useful in the range of about 650-1000 nm (i.e., in the near infrared (“NIR”)). For example, pyropheophorbide and bacteriochloin are useful in about the 650-900 nm range.

A photoactivatable molecule is a chemical compound that upon exposure to photoactivating light is activated, releasing a singlet oxygen species. The photoactivatable molecules of the target-binding agents disclosed herein can be any of the variety of synthetic and naturally occurring photosensitizing agents known in the art, including but not limited to, porphyrins; chlorins; bacteriochlorins; isbacteriochlorins; phthalocyanines; napthalocyanines; porphycenes; porphycyanines; tetra-macrocyclic compounds; poly-macrocyclic compounds; pyropheo-phorbides; pentaphyrin; sapphyrins; texaphyrins; metal complexes; tetrahydrochlorins; phonoxazine dyes; phenothiazines; chaloorganapyrylium dyes; rhodamines; fluorescenes; azoporphyrins; benzochlorins; purpurins; chlorophylls; verdins; triarylmethanes; angelicins; chalcogenapyrillium dyes; chlorins; chlorophylls; coumarins; cyanines; ceratin daunomycin; daunomycinone; 5-iminodauno-mycin; doxycycline; furosemide; gilvocarcin M; gilvocarcin V; hydroxy-chloroquine sulfate; lumidoxycycline; mefloquine hydrochloride; mequitazine; merbromin (mercurochrome); primaquine diphosphate; quinacrine dihydrochloride; quinine sulfate; and tetracycline hydrochloride; certain flavins and related compounds such as alloxazine; flavin mononucleotide; 3-hydroxyflavone; limichrome; limiflavin; 6-methylalloxazine; 7-methylalloxazine; 8-methylalloxazine; 9-methylalloxazine; 1-methyl limichrome; methyl-2-methoxybenzoate; 5-nitrosalicyclic acid; proflavine; and riboflavin; metallo-porphyrins; metallophthalocyanines; methylene blue derivatives; naphthalmides; naphthalocyanines; pheophorbides; pheophytins; photosensitizer dimers and conjugates; phthalocyanines; porphycenes; quinones; retinoids; rhodamines; thiophenes; verdins; vitamins; and xanthene dyes. Generally, any polypyrrolic macrocyclic photosensitive compound that is hydrophobic can be used.

The release of reactive oxygen species, such as singlet oxygen, may disrupt active cellular metabolism and cause photodamage by apoptosis. Some photoactivatable molecules, such as phthalocyanines have been shown to cause necrosis by a metabolism-independent mechanism (See, e.g., Prasad, Introduction to Biophotonics, John Wiley & Sons, Inc. Hoboken, N.J. (2003), which is incorporated herein by reference). Oxidative degradation of membrane lipids can produce loss of membrane integrity resulting in impairment of membrane transport, rupturing of membrane, increased permeability, and crosslinking/inactivation of membrane associated polypeptides such as receptors, enzymes and ion channels. Chlorin, benzoporphyrin, and some phthalocyanine photosensitizers have been shown to cause damage to lysosomes. (See, e.g., Prasad, Intro. to Biophotonics, John Wiley & Sons, Inc. Hoboken, N.J. (2003), which is incorporated herein by reference)

Photoexcitation of the photoactivatable molecules by linear absorption (as opposed to excitation by a nonlinear, two-photon absorption) does not require a high peak power or a coherent light source. As such, tungsten and/or mercury or xenon arc lamps may be used to activate the photoactivatable molecules. Alternatively, lasers may be used for this purpose. Examples include a dye laser with rhodamine B as lasing medium and pumped by an argon-ion laser or an intracavity KTP-doubled Nd:Vanadate laser, both producing a CW dye laser output in the range of 1-4 W. Alternatively, pulse laser sources providing high repetition rates in the kilohertz range may be used and include gold vapor lasers, copper-pumped dye lasers, and quasi-CW Q-switched Nd:YAG laser-pumped dye lasers. In some instances, a solid-state diode laser may be used with CW and quasi-CW powers in the range of 1-4 W with a single emitter source in the range of 780-850 nm. Other laser sources include, but are not limited to tunable solid-state lasers such a the Ti:sapphire laser (690-1100 nm) and the Alexandrite lasers (720-800 nm) (See, e.g., Prasad, Introduction to Biophotonics, John Wiley & Sons, Inc. Hoboken, N.J. (2003), which is incorporated herein by reference).

The photoactivatable molecule itself may be monitored by quantitative fluorometry or reflectance spectophotometry. Activation of the photoactivatable molecules may be assessed, for example, by measuring singlet oxygen production at about 1270 nm (See, e.g., Lee et al., “Optical Methods for Tumor Treatment and Detection: Mechanisms and Techniques in Photodynamic therapy,” XV Biomedical Optics (BiOS) Symposium, San Jose, Calif. (2006), which is incorporated herein by reference).

A modified red blood cell may be loaded with a photosensitive reagent such as, for example, a derivative of hematoporphyrin and subsequently irradiated to release a therapeutic agent (See, e.g., Flynn et al., Cancer Lett. 82:225-229 (1994), which is incorporated herein by reference). For example, modified red blood cells are suspended in a physiological buffer such as Ringer's Lactate Solution or saline solution with 5% dextrose (w/v) to which is added hematoporphyrin at a concentration of about 250 μg/ml. The cell suspension is incubated at 4° C. for 90 min and subsequently washed with the physiological buffer. The cells may be loaded with a therapeutic agent before, after, or concomitant with hematophorphyrin loading. The modified red blood cells maybe irradiated with a 10 mW output HeNe laser, for example, to induce disruption of modified red blood cells and release of the therapeutic agent (See, e.g., Flynn et al., Cancer Lett. 82:225-229 (1994), which is incorporated herein by reference).

Examples of some classes of photoactivatable molecules include, but are not limited to, angelicins, chalcogenapyrillium dyes, chlorins, chlorophylls, coumarins, cyanines, ceratin daunomycin; daunomycinone; 5-iminodauno-mycin; doxycycline; furosemide; gilvocarcin M; gilvocarcin V; hydroxy-chloroquine sulfate; lumidoxycycline; mefloquine hydrochloride; mequitazine; merbromin (mercurochrome); primaquine diphosphate; quinacrine dihydrochloride; quinine sulfate; and tetracycline hydrochloride, certain flavins and related compounds such as alloxazine; flavin mononucleotide; 3-hydroxyflavone; limichrome; limiflavin; 6-methylalloxazine; 7-methylalloxazine; 8-methylalloxazine; 9-methylalloxazine; 1-methyl limichrome; methyl-2-methoxybenzoate; 5-nitrosalicyclic acid; proflavine; and riboflavin, metallo-porphyrins, metallophthalocyanines, methylene blue derivatives, naphthalmides, naphthalocyanines, pheophorbides, pheophytins, photosensitizer dimers and conjugates, phthalocyanines, porphycenes, porphyrins, psoralens, purpurins, quinones, retinoids, rhodamines, thiophenes, verdins, vitamins and xanthene dyes (Redmond and Gamlin, Photochem. Photobiol., 70(4):391-475 (1999), which is incorporated herein by reference).

1. Porphyrins

Some non-limiting examples of porphyrins include 5-azaprotoporphyrin dimethylester; bis-porphyrin; coproporphyrin III; coproporphyrin III tetramethylester; deuteroporphyrin; deuteroporphyrin IX dimethylester; diformyldeutero-porphyrin IX dimethylester; dodecaphenylporphyrin; hematoporphyrin; hematoporphyrin; hematoporphyrin; hematoporphyrin; hematoporphyrin; hematoporphyrin; hematoporphyrin; hematoporphyrin; hematoporphyrin IX; hematoporphyrin monomer; hematoporphyrin dimer; hematoporphyrin derivative; hematoporphyrin derivative; hematoporphyrin derivative; hematoporphyrin derivative A; hematoporphyrin IX dihydrochloride; hematoporphyrin dihydrochloride; hematoporphyrin IX dimethylester; haematoporphyrin IX dimethylester; mesoporphyrin dimethylester; mesoporphyrin IX dimethylester; monoformyl-monovinyl-deuteroporphyrin IX dimethylester; monohydroxyethylvinyl deuteroporphyrin; 5,10,15,20-tetra(o-hydroxyphenyl)porphyrin; 5,10,15,20-tetra(m-hydroxyphenyl)porphyrin; 5,10,15,20-tetralds-(m-hydroxyphenyl)-porphyrin; 5,10,15,20-tetra(p-hydroxyphenyl)porphyrin; 5,10,15,20-tetralds (3-methoxyphenye-porphyrin; 5,10,15,20-tetrakis (3,4-dimethoxyphenyl)porphyrin; 5,10,15,20-tetrakis (3,5-dimethoxyphenyl)porphyrin; 5,10,15,20-tetralds (3,4,5-trimethoxyphenyl)porphyrin; 2,3,7,8,12,13,17,18-oetaethyl-5,10,15,20-tetraphenylporphyrin; Photofrin®; Photofrin II; porphyrin c; protoporphyrin; protoporphyrin IX; protoporphyrin dimethylester; protoporphyrin IX dimethylester, protoporphyrin propylaminoethylformamide iodide; protoporphyrin N,N-dimethylaminopropyl-formamide; protoporphyrin propylaminopropylformamide iodide; protoporphyrin butylformamide; protoporphyrin N,N-dimethylamino-formamide; protoporphyrin fonnamide; sapphyrin 13,12,13,22-tetraethyl-2,7,18,23 tetramethyl sapphyrin-8,17-dipropanol; sapphyrin 23,12,13,22-tetraethyl-2,7,18,23 tetramethyl sapphyrin-8-monoglycoside; sapphyrin 3; meso-tetra-(4-N-carboxyphenyl)-porphine; tetra-(3-methoxyphenyl)-porphine; tetra-(3-methoxy-2,4-difluorophenyl)-porphine; 5,10,15,20-tetrakis(4-N-methylpyridyl)porphine; meso-tetra-(4-N-methylpyridyl)-porphine tetrachloride; meso-tetra(4-N-methylpyridyl)-porphine; meso-tetra-(3-N-methylpyridyl)-porphine; meso-tetra-(2-N-methylpyridyl)-porphine; tetra(4-N,N,N-trimethylanilinium)porphine; meso-tetra-(4-N,N,N″-trimethylamine-phenyl)porphine tetrachloride; tetranaphthaloporphyrin; 5,10,15,20-tetraphenylporphyrin; tetraphenylporphyrin; meso-tetra-(4-N-sulfonatophenyl)-porphine; tetraphenylporphine tetrasulfonate; meso-tetra(4-sulfonatophenyl)-porphine; tetra(4-sulfonatophenyl)porphine; tetraphenylporphyrin sulfonate; meso-tetra(4-sulfonatophenyl)porphine; tetrakis(4-sulfonatophenyl)porphyrin; meso-tetra(4-sulfonatophenyl)porphine; meso(4-sulfonatophenyl)porphine; meso-tetra(4-sulfonatophenyl)porphine; tetrakis(4-Sulfonatophenyl)porphyrin; meso-tetra(4-N-trimethylanilinium)-porphine; uroporphyrin; uroporphyrin I; uroporphyrin IX; and uroporphyrin I.

2. Metalloporphyrins

Some non-limiting examples of metalloporphyrins include cobalt meso-tetra-(4-N-methylpyridyl)-porphine; cobalt (II) meso(4-sulfonatophenyl)-porphine; copper hematoporphyrin; copper meso-tetra-(4-N-methylpyridyl)-porphine; copper (II) meso(4-sulfonatophenyl)-porphine; Europium (III) dimethyltexaphyrin dihydroxide; gallium tetraphenylporphyrin; iron meso-tetra(4-N-methylpyridyl)-porphine; lutetium (III) tetra(N-methyl-3-pyridyl)-porphyrin chloride; magnesium (II) meso-diphenyl tetrabenzoporphyrin; magnesium tetrabenzoporphyrin; magnesium tetraphenylporphyrin; magnesium (II) meso(4-sulfonatophenyl)-porphine; magnesium (II) texaphyrin hydroxide metalloporphyrin; magnesium meso-tetra-(4-N-methylpyridyl)-porphine; manganese meso-tetra-(4-N-methyl-pyridyl)-porphine; nickel meso-tetra(4-N-methylpyridyl)-porphine; nickel (II) meso-tetra(4-sulfonatophenyl)-porphine; palladium (II) meso-tetra-(4-N-methylpyridyl)-porphine; palladium meso-tetra-(4-N-methylpyridyl)-porphine; palladium tetraphenylporphyrin; palladium (II) meso(4-sulfonatophenyl)-porphine; platinum (II) meso(4-sulfonatophenyl)-porphine; samarium (II) dimethyltexaphyrin dihydroxide; silver (II) meso(4-sulfonatophenyl)-porphine; tin (IV) protoporphyrin; tin meso-tetra-(4-N-methylpyridyl)-porphine; tin meso-tetra(4-sulfonatophenyl)-porphine; tin (IV) tetrakis(4-sulfonatophenyl)porphyrin dichloride; cadmium (II) chlorotexaphyrin nitrate; cadmium (II) meso-diphenyl tetrabenzoporphyrin; cadmium meso-tetra-(4-N-methylpyridyl)-porphine; cadmium (II) texaphyrin; cadmium (II) texaphyrin nitrate; zinc (II) 15-aza-3,7,12,18-tetramethyl-porphyrinato-13,17-diyl-dipropionic acid-dimethylester; zinc (II) chlorotexaphyrin chloride; zinc coproporphyrin III; zinc (II) 2,11,20,30-tetra-(1,1-dimethyl-ethyl)tetranaphtho (2,3-b:2′,3′-g:2″3″-1:-2′″3′″-q)porphyrazine; zinc (II) 2-(3)-pyridyloxy)benzo[b]-10,19,28-tri(1,1-dimethylethyl)trinaphtho[2′,3′- -g:2″3″1::2′″,3′″-q]porphyrazine; zinc (II) 2,18-bis-(3-pyridyloxy)dibenzo[b,1]-10,26-di(1,1-dimethyl-ethyl)dinaphtho-[2′,3′-g:2′″,3′″-q]porphyrazine; zinc (II) 2,9-bis-(3-pyridyloxy)dibenzo[b,g]-17,26-di(1,1-dimethyl-ethyl)dinaphtho[-2″,3″-1:2′″,3′″-q]porphyrazine; zinc (II) 2,9,16-tris-(3-pyridyloxy)tribenzo[b,g,1]-24=(1,1-dimethyl-ethyl)naphtho[-2′″,3′″-q]porphyrazine; zinc (II) 2,3-bis-(3-pyridyloxy)benzo[b]-10,19,28-tri(1,1-dimethyl-ethyl)trinaphtho-[2′,3′-g:2″,3″1:2′″,3′″-q]porphyrazine; zinc (II) 2,3,18,19-tetrakis-(3-pyridyloxy)dibenzo[b,1]-10,26-di(1,1-dimethyl-ethyl-)trinaphtho[2′,3′-g:2′″,3′″-q]porphyrazine; zinc (II) 2,3,9,10-tetralcis-(3-pyridyloxy)dibenzo[b,g]-17,26-di(1,1-dimethyl-ethyl)-dinaphtho[2″,3″-1:2′″,3′″-q]porphyrazine; zinc (II) 2,3,9,10,16,17-hexalcis-(3-pyridyloxy)tribenzo[b,g,1]-24(1,1-dimethyl-ethy-1)naphtho[2′″,3′″-q]porphyrazine; zinc (II) 2-(3-N-methyl)pyridyloxy)benzo[b]-10,19,28-tri(1,1-dimethyl-ethyl)trinaph-tho[2′,3′-g:2″,3″1:2′″,3′″-q]porphyrazine monoiodide; zinc (II) 2,18-bis-(3-(N-methyl)pyridyloxy)dibenzo[b,1]-10,26-di(1,1-dimethylethyl)-dinaphtho[2′,3′-g:2′″,3′″-q]porphyrazine diiodide; zinc (II) 2,9-bis-(3-(N-methyl)pyridyloxy)dibenzo[b,g]-17,26-di(1,1-dimethylethyl)d-inaphtho[2″,3″-1:2′″,3′″-q]porphyrazine diiodide; zinc (II) 2,9,16-tris-(3-(N-methyl-pyridyloxy)tribenzo[b,g,1]-24-(1,1-dimethylethyl-)naphtho[2′″,3′″-q]porphyrazine triiodide; zinc (II) 2,3-bis-(3-(N-methyl)pyridyloxy)benzo[b]-10,19,28-tri(1,1-dimethylethyl)t-rinaphtho[2′,3′-g:2″,3″-1:2′″,3′″-q]porphyrazine diiodide; zinc (II) 2,3,18,19-tetrakis-(3-(N-methyl)pyridyloxy)dibenzo[b,1]-10,26-di(1,1-dimethyl)dinaphtho[2′,3′-g:2′,3′″-q]porphyrazine tetraiodide; zinc (II) 2,3,9,10-tetralkis-(3-(N-methyl)pyridyloxy)dibenzo[g,g]-17,26-di(1,1-dimet-hylethyl)dinaphtho[2″,341-1:2′″,3′″-q]porphyrazine tetraiodide; zinc (II) 2,3,9,10,16,17-hexakis-(3-(N-methyl)pyridyloxy)tribenzo[b,g,1]-24-(1-,1-dimethylethyl)naphtho[2′″,3′″-q]porphyrazine hexaiodide; zinc (II) meso-diphenyl tetrabenzoporphyrin; zinc (II) meso-triphenyl tetrabenzoporphyrin; zinc (II) meso-tetralds(2,6-dichloro-3-sulfonatophenyl)porphyrin; zinc (II) meso-tetra-(4-N-methylpyridyl)-porphine; zinc (II) 5,10,15,20-meso-tetra(4-octyl-phenylpropynyl)-porphine; zinc porphyrin c; zinc protoporphyrin; zinc protoporphyrin IX; zinc (II) meso-triphenyl-tetrabenzoporphyrin; zinc tetrabenzoporphyrin; zinc (II) tetrabenzoporphyrin; zinc tetranaphthaloporphyrin; zinc tetraphenylporphyrin; zinc (II) 5,10,15,20-tetraphenylporphyrin; zinc (II) meso (4-sulfonatophenyl)-porphine; and zinc (II) texaphyrin chloride.

3. Pheophorbides

Some non-limiting examples of pheophorbides include pheophorbide a; methyl 13-1-deoxy-20-formyl-7,8-vic-dihydro-bacterio-meso-pheophorbide a; methyl-2-(1-dodecyloxyethyl)-2-devinyl-pyropheophorbide a; methyl-2-(1-heptyl-oxyethyl)-2-devinyl-pyropheophorbide a; methyl-2-(1-hexyl-oxyethyl)-2-devinyl-pyropheophorbide a; methyl-2-(1-methoxy-ethyl)-2-devinyl-pyropheophorbide a; methyl-2-(1-pentyl-oxyethyl)-2-devinyl-pyropheophorbide a; magnesium methyl bacteriopheophorbide d; methyl-bacteriopheophorbide d; and pheophorbide.

4. Psoralens

Some non-limiting examples of psoralens include psoralen; 5-methoxypsoralen; 8-methoxy-psoralen; 5,8-dimethoxypsoralen; 3-carbethoxypsoralen; 3-carbethoxy-pseudopsoralen; 8-hydroxypsoralen; pseudopsoralen; 4,5′,8-trimethyl-psoralen; allopsoralen; 3-aceto-allopsoralen; 4,7-dimethyl-allopsoralen; 4,7,4′-trimethyl-allopsoralen; 4,7,5′-trimethyl-allopsoralen; isopseudopsoralen; 3-acetoisopseudopsoralen; 4,5′-dimethyl-isopseudo-psoralen; 5′,7-dimethyl-isopseudopsoralen; pseudoisopsoralen; 3-aceto-seudoisopsoralen; 3/4′,5′-trimethyl-aza-psoralen; 4,4′,8-trimethyl-5′-amino-methylpsoralen; 4,4′,8-trimethyl-phthalamyl-psoralen; 4,5′,8-trimethyl-4′-aminomethyl psoralen; 4,5′,8-trimethyl-bromopsoralen; 5-nitro-8-methoxy-psoralen; 5′-acetyl4,8-dimethyl-psoralen; 5′-aceto-8-methyl-psoralen; and 5′-aceto-4,8-dimethyl-psoralen. Examples of purpurins include octaethylpurpurin; octaethylpurpurin zinc; oxidized octaethylpurpurin; reduced octaethylpurpurin; reduced octaethylpurpurin tin; purpurin 18; purpurin-18; purpurin-18-methyl ester; purpurin; tin ethyl etiopurpurin I; Zn(II) aetio-purpurin ethyl ester; and zinc etiopurpurin.

5. Quinones

Some non-limiting examples of quinones include 1-amino-4,5-dimethoxy anthraquinone; 1,5-diamino-4,8-dimethoxy anthraquinone; 1,8-diamino-4,5-dimethoxy anthraquinone; 2,5-diamino-1,8-dihydroxy anthraquinone; 2,7-diamino-1,8-dihydroxy anthraquinone; 4,5-diamino-1,8-dihydroxy anthraquinone; mono-methylated 4,5- or 2,7-diamino-1,8-dihydroxy anthraquinone; anthralin (keto form); anthralin; anthralin anion; 1,8-dihydroxy anthraquinone; 1,8-dihydroxy anthraquinone (Chrysazin); 1,2-dihydroxy anthraquinone; 1,2-dihydroxy anthraquinone (Alizarin); 1,4-dihydroxy anthraquinone (Quinizarin); 2,6-dihydroxy anthraquinone; 2,6-dihydroxy anthraquinone (Anthraflavin); 1-hydroxy anthraquinone (Erythroxy-anthraquinone); 2-hydroxy-anthraquinone; 1,2,5,8-tetra-hydroxy anthraquinone (Quinalizarin); 3-methyl-1,6,8-trihydroxy anthraquinone (Emodin); anthraquinone; anthraquinone-2-sulfonic acid; benzoquinone; tetramethyl benzoquinone; hydroquinone; chlorohydroquinone; resorcinol; and 4-chlororesorcinol.

6. Retinoids

Some non-limiting examples of retinoids include all-trans retinal; C₁₇ aldehyde; C₂₂ aldehyde; 11-cis retinal; 13-cis retinal; retinal; and retinal palmitate.

7. Rhodamines

Some non-limiting examples of rhodamines include 4,5-dibromo-rhodamine methyl ester; 4,5-dibromo-rhodamine n-butyl ester; rhodamine 101 methyl ester; rhodamine 123; rhodamine 6G; rhodamine 6G hexyl ester; tetrabromo-rhodamine 123; and tetramethyl-rhodamine ethyl ester.

8. Other Photoactivatable Molecules

Other non-limiting examples of photoactivatable molecules that may be useful in the target-binding agents are bacteriochlorophyll-A derivatives, described in U.S. Pat. Nos. 5,171,741 and 5,173,504; photosensitizing Diels-Alder porphyrin derivatives, described in U.S. Pat. No. 5,308,608; porphyrin-like compounds, described in U.S. Pat. Nos. 5,405,957, 5,512,675, and 5,726,304; imines of porphyrin and porphyrin derivatives, as described in U.S. Pat. Nos. 5,424,305 and 5,744,598; alkyl ether analogs of benzoporphyrin derivatives, as described in U.S. Pat No. 5,498,710; purpurin-18, bacteriopurpurin-18 and related compounds, as described in U.S. Pat. No. 5,591,847; meso-substituted chorins, isobacteriochlorins and bacteriochlorins, as described in U.S. Pat. No. 5,648,485; meso-substituted tetramacrocyclic compounds, as described in U.S. Pat. No. 5,703,230; carbodiimide analogs of chlorins and bacteriochlorins, as described in U.S. Pat. No. 5,770,730; meso-substituted chlorins, isobacteriochlorins and bacteriochlorins, as described in U.S. Pat. No. 5,831,088; polypyrrolic macrocycles from meso-substituted tripyrrane compounds, described in U.S. Pat. Nos. 5,703,230, 5,883,246, and 5,919,923; isoimides of chlorins and bacteriochlorins, described in U.S. Pat. No. 5,864,035; alkyl ether analogs of chlorins having an N-substituted imide ring, as described in U.S. Pat. No. 5,952,366; ethylene glycol esters, described in U.S. Pat. No. 5,929,105; carotene analogs of porphyrins, chlorins and bacteriochlorins, as described in U.S. Pat. No. 6,103,751; fatty acid ester derivatives of porphyrin, chlorin, or bacteriochlorin, as described in U.S. Pat. No. 6,245,811; indium photosensitizers, as described in U.S. Pat. No. 6,444,194; porphyrins, chlorins, bacteriochlorins, and related tetrapyrrolic compounds described in U.S. Pat. No. 6,534,040; 1,3-propane diol ester and ether derivatives of porphyrins, chlorins and bacteriochlorins, as described in U.S. Pat. No. 6,555,700; trans beta substituted chlorins, as described in U.S. Pat. No. 6,559,374; and palladium-substituted bacteriochlorophyl derivatives, as described in U.S. Pat. No. 6,569,846; and the photosensitizer entities disclosed in Wilson et al., (Curr. Micro. 25:77-81 (1992)) and in Okamoto et al., (Lasers in Surg. Med. 12:450-458 (1992)), each of which is incorporated herein by reference. Generally any hydrophobic or hydrophilic photosensitizing agent, that absorbs in the ultra-violet, visible and infra-red spectroscopic ranges, would be useful in the disclosed conjugates.

D. Quencher Molecules

In various embodiments, the target-binding agents include a quencher molecule. In an embodiment, a light quencher is provided to prevent activation of the photoactivatable molecule if the targeting composition is not bound to a target molecule. Alternatively, the quencher may capture singlet oxygen from the photoactivatable molecule in situations where the target-binding agent is not bound to the target.

In an embodiment, the quencher molecule quenches the excited state of the photoactivatable molecule. For example, upon binding of the target-binding agent to its target, the three dimensional structure of the target-binding agent is altered in such a way that the quenching agent is no longer positioned close enough to quench the excited state of the photoactivatable molecule, thus allowing the photoactivatable molecule to function as required for generation of singlet oxygen. The singlet oxygen is then available to destroy the target or lyse the modified red blood cell. The quenching agent serves to prevent the generation of false positive signals from the photoactivatable molecule when it is not bound to the target.

In a specific embodiment, the photoactivatable molecule is a porphyrin or porphyrin derivative tetrapyrrole that includes a metal atom in its central coordination cavity and the quencher comprises one or more suitable functional groups that coordinate to the axial position of the metal coordinated within the photoactivatable molecule. The target recognition moiety is positioned in the agent in such a way that the interaction of the target recognition moiety with the target disrupts the association of the axial ligand to the metal, releasing the quenching agent and allowing the porphyrin or porphyrin derivative tetrapyrrole to be activated when irradiated.

In an embodiment, the quencher molecule is a light quencher, which prevents light of a suitable wavelength from exciting the photoactivable molecule. For instance, the quencher may absorb photons of a particular wavelength before those photons activate the photoactivatable molecule. Suitable light quenchers may include 4-(4′-dimethylamino-phenylazo)benzoic acid (Dabcyl) or dark quenchers, such as black hole quenchers sold under the tradename “BHQ” (e.g., BHQ-0, BHQ-1, BHQ-2, and BHQ-3, Biosearch Technologies, Novato, Calif.). Dark quenchers also may include quenchers sold under the tradename “QXL™” (Anaspec, San Jose, Calif.). Dark quenchers also may include DNP-type non-fluorophores that include a 2,4-dinitrophenyl group.

In an embodiment, the quencher molecule is an antioxidant which captures singlet oxygen produced by the photoactivatable molecule before it can cause damage to Surround cells or tissues. Suitable quenchers for singlet oxygen include, but are not limited to, glutathione, trolox, flavonoids, vitamin C, vitamin E, cysteine and ergothioneine and other non-toxic quenchers.

E. Molecules

In an embodiment, the red blood cells may be modified with fusion molecules or fusogens known to facilitate fusion with other cells. Upon fusion, the modified red blood cell may release its loaded content such as, for example, an anti-cancer therapeutic agent or a photosensitive reagent. For instance, breast cancer cells have been shown to express an endogenous retroviral envelope protein, syncytin-1, that enables the tumor cells to fuse in vivo with endothelial cells expressing a corresponding D-type retroviral receptor, the Na+-dependent neutral amino acid transporter ASCT2 (See, e.g., Larsson et al., Scientific World Journal 7:1193-1197 (2007), which is incorporated herein by reference). Syncytin-1 is also expressed by endometrial carcinomas. As such, red blood cells may be modified with a syncytin-1 interacting receptor such as, for example, ASCT2 that would enable the modified red blood cells to fuse with cancer cells. For example, cDNA encoding human ASCT2 may be cloned using sequence information available in NCBI/GenBank (See, e.g., accession number NP_{—}005619).

Alternatively, cDNA encoding human ASCT2 may be acquired from a commercial source (e.g., OriGene Technologies, Inc., Rockville, Md., USA). The cDNA is cloned into an appropriate expression vector and subsequently transfected into cultured hematopoietic stem cells. Alternatively, the cDNA encoding ASCT2 may be transcribed to generate mRNA which is subsequently introduced into isolated reticulocytes as described above.

Methods of making compositions described herein. In an embodiment, a method of making at least one artificial antigen presenting cell includes joining at least two members of at least one antigen presenting cell complex, and optionally displaying the at least one antigen presenting cell complex on the surface of the vehicle of the composition. As described herein, in an embodiment, the vehicle includes at least one of a biological cell, lipid surface, polymeric vehicle, chemical emulsion, phase separation, device, micelle, chip, red blood cell ghost, cerasome, liposome, lipid bilayer, lipid monolayer, lipid multilayer, platelet, exosome, lipid droplet, or other vehicle. In an embodiment, the at least one artificial antigen presenting cell complex is internalized by the vehicle (e.g., by electroporation, endocytosis, cellular swelling, etc.). In an embodiment, the at least one artificial antigen presenting cell complex remains internalized until the vehicle is ruptured or dissociates to release or expose the complex. In an embodiment, the at least one artificial antigen presenting cell complex is displayed on the inner or outer surface of the vehicle.

### II. Assembly of the Target-Binding Agents

A. Attachment of a Target Recognition Moiety to a Photoactivatable Molecule and a Quencher Molecule

In an embodiment, the target recognition moiety of the target-binding agent is conjugated to a photoactivable molecule and a quencher molecule. Upon binding of the target recognition moiety to the target molecule, the quencher molecule is released or otherwise separated from the photoactivateable molecule. In the “unquenched” state, the photoactivatable molecule may be activated by light of a suitable wavelength. The conjugation of these molecules is typically by way of attachment sites. Most attachments are conveniently effected via sulfhydryl or amine interactions. Synthetic and commercial alternatives are available depending on the selected photoactivable molecule, or quencher molecule. The distance between the photoactivatable molecule and the quencher molecule is selected so that interaction of the target recognition moiety results in repositioning of the quencher molecule. If the photoactivatable molecule and the quencher are too close, then interaction of the target recognition moiety with the target may not end quenching of photoactivatable molecule. If the distance between the photoactivatable molecule and the quencher molecule is too great, then the quencher molecule may not prevent all electromagnetic radiation from reaching the photoactivatable molecule. The distances can be determined by any method, such as by calculation or empirically.

Techniques in synthetic chemistry provide methods for the attachment of photoactivatable molecule and/or quencher molecule to the target recognition moiety. For example, synthetic linkage techniques are known that allow incorporation of various types of molecules, including a photoactivatable molecule and an quencher molecule within an oligonucleotide (See U.S. Pat. No. 4,996,143, which is incorporated herein by reference). There is extensive guidance in the literature for derivatizing photoactivatable and quencher molecules for covalent attachment via readily available reactive groups that can be added to a molecule. The diversity and utility of chemistries available for conjugating molecules and surfaces is exemplified by the extensive body of literature on preparing nucleic acids derivatized with fluorophores. See, for example, Ullhman et al., U.S. Pat. No. 3,996,345 and Khanna et al., U.S. Pat. No. 4,351,760, each of which is incorporated herein by reference.

The target-binding agents disclosed herein can be conjugated by using a coupling agent. Any bond which is capable of linking the components such that they are stable under physiological conditions for the time needed for administration and treatment is suitable, but covalent linkages are preferred. The link between two components may be direct, e.g., where a photoactivatable molecule is linked directly to a target recognition moiety, or indirect, e.g., where a photoactivatable molecule is linked to a linking component and that linking component being linked to the target recognition moiety.

A coupling agent should function under conditions of temperature, pH, salt, solvent system, and other reactants that substantially retain the chemical stability of the photoactivatable molecule, the quencher molecule and the target recognition moiety. Coupling agents should link the component moieties stably, but such that there is only minimal or no denaturation or deactivation of the photoactivatable molecule, quencher molecule or the target recognition moiety. Many coupling agents react with an amine and a carboxylate, to form an amide, or an alcohol and a carboxylate to form an ester. Coupling agents are known in the art (See, e.g., Bodansky, Principles of Peptide Synthesis, 2nd ed, John Wiley, NY (1991), and Greene & Wuts, Protective Groups in Organic Synthesis, 2nd ed, John Wiley; NY (1991), each of which is incorporated herein by reference). Representative combinations of such groups are amino with carboxyl to form amide linkages, or carboxy with hydroxy to form ester linkages or amino with alkyl halides to form alkylamine linkages, or thiols with thiols to form disulfides, or thiols with maleimides or alkyl halides to form thioethers. Obviously, hydroxyl, carboxyl, amino and other functionalities, where not present may be introduced by known methods.

The target-binding agents provided herein can be prepared by coupling the photoactivatable molecule to a target recognition moiety, such as an antibody, by cleaving an available ester moiety on the photoactivatable molecule and coupling the compound via peptide linkages to an antibody through an N terminus, or by other methods known in the art. A variety of coupling agents, including cross-linking agents, can be used for covalent conjugation. Examples of cross-linking agents include N,N′-dicyclohexylcarbodiimide (DCC), N-succinimidyl-S-acetyl-thioacetate (SATA), N-succinimidyl-3-(2-pyridyldithio)propionate (SPDP); ortho-phenylene-dimaleimide (o-PDM), and sulfosuccinimidyl 4N-maleimido-methyl)-cyclohexane-1-carboxylate (sulfo-SMCC). See, e.g., Karpovsky et al., J Exp. Med. 160:1686 (1984); and Liu M A et al., Proc. Natl. Acad. Sci. USA 82: 8648 (1985), each of which is incorporated herein by reference. Other methods include those described by Brennan et al., Science 229: 81-83 (1985) and Glennie et al., J. Immunol. 139: 2367-2375 (1987), each of which is incorporated herein by reference. A large number of coupling agents for peptides and proteins, along with buffers, solvents, and methods of use, are described in the Pierce Chemical Co. catalog, pages O-90 to O-110 (1995, Pierce Chemical Co., 3747 N. Meridian Rd., Rockford Ill., 61105, U.S.A.), which is incorporated herein by reference.

For example, DCC is a useful coupling agent that cane be used to promote coupling of the alcohol NHS to chlorin e6 in DMSO forming an activated ester which can be cross-linked to polylysine. DCC is a carboxy-reactive cross-linker commonly used as a coupling agent in peptide synthesis. Another useful cross-linking agent is SPDP, a heterobifunctional cross-linker for use with primary amines and sulfhydryl groups. SPDP has a molecular weight of 312.4, a spacer arm length of 6.8 angstroms, is reactive to NHS-esters and pyridyldithio groups, and produces cleavable cross-linking such that, upon further reaction, the agent is eliminated so the photoactivatable molecule can be linked directly to a linking component or target recognition moiety. Other useful conjugating agents are SATA for introduction of blocked SH groups for two-step cross-linking, which is deblocked with hydroxylamine-HCl, and sulfo-SMCC, reactive towards amines and sulfhydryls. Other cross-linking and coupling agents are also available from Pierce Chemical Co. Additional compounds and processes, particularly those involving a Schiff base as an intermediate, for conjugation of proteins to other proteins or to other compositions, for example to reporter groups or to chelators for metal ion labeling of a protein, are disclosed in EPO 243,929 A2 (published Nov. 4, 1987), which is incorporated herein by reference.

Reactive Groups. The photoactivatable molecule or target recognition moiety can be conjugated, directly or through a linking component, to the quencher molecule using reactive groups, either on the donor molecule or on the acceptor molecule or the targeting moiety. For example, molecules that contain carboxyl groups can be joined to lysine-amino groups in the target polypeptides either by preformed reactive esters (such as N-hydroxy succinimide ester) or esters conjugated in situ by a carbodiimide-mediated reaction. The same applies to molecules that contain sulfonic acid groups, which can be transformed to sulfonyl chlorides which react with amino groups. Molecules that have carboxyl groups can be joined to amino groups, such as on a polypeptide, by an in situ carbodiimide method. Molecules can also be attached to hydroxyl groups of serine or threonine residues or to sulfhydryl groups of cysteine residues.

Methods of joining components of a target-binding agent can use heterobifunctional cross-linking reagents. These agents bind a functional group in one chain and to a different functional group in the second chain. These functional groups typically are amino, carboxyl, sulfhydryl, and aldehyde. There are many permutations of appropriate moieties which will react with these groups and with differently formulated structures, to conjugate them together. (See Merrifield et al., Ciba Found Symp. 186: 5-20 (1994), which is incorporated herein by reference).

The photoactivatable molecule of the target-binding agent may be optionally functionalized so as to include a linking component which allows the photoactivatable molecule to be linked to a target recognition moiety, such as an analyte, antigen, antibody or other molecule. For example, the linking component may include, but is not limited to, an oligonucleotide, a polynucleotide, a nucleic acid, an oligosaccharide, a polysaccharide or a diaminoalkane linking species, such as 1,3-diaminopropane. A variety of linking components which are suited to this purpose have been described. For example, see Kricka, Ligand-Binder Assays; Labels and Analytical Strategies, pp. 15-51, Marcel Dekker, Inc., New York, N.Y. (1985), which is incorporated herein by reference). The photoactivatable molecule is linked to the linking component and the linking component is linked to the analyte, antigen, antibody or other molecule using conventional techniques.

Reactive Groups and Reactions. Reactive groups and classes of reactions useful in preparing the disclosed conjugates are generally those that are well known in the art of bioconjugate chemistry. Classes of reactions include those that proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction). These and other useful reactions are discussed in, for example, Morrison et al., Organic Chemistry, 4th Ed., Allyn and Bacon, Inc. (1983), and Hermanson, Bioconjugate Techniques, Academic Press, San Diego (1996), each of which is incorporated herein by reference.

For example, useful reactive functional groups include: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups, which can be converted to esters, ethers, aldehydes, etc.; (c) haloalkyl groups, wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups, which are capable of participating in Diels-Alder reactions such as, for example, maleimido groups; (e) carbonyl groups, such that subsequent derivatization is possible via formation of carbonyl derivatives such as, for example, imines, hydrazones, semicarbazones or oximes, or via such mechanisms as Grignard addition or alkyllithium addition; (f) sulfonyl groups for subsequent reaction with amines, for example, to form sulfonamides; (g) thiol groups, which can be converted to disulfides or reacted with acyl halides; (h) amine or sulfhydryl groups, which can be, for example, acylated, alkylated or oxidized; (i) alkenes, which can undergo, for example, cycloadditions, acylation, Michael addition, etc; (j) epoxides, which can react with, for example, amines and hydroxyl compounds; and (k) phosphoramidites and other standard functional groups useful in nucleic acid synthesis.

B. Placement of the Photoactivatable Molecule and Quencher Molecule

The photoactivatable molecule and quencher molecule of the target-binding agents disclosed herein are positioned to be in a configuration so that the agent is in a “quenched state” when it is not interacting with a target molecule. When the agent interacts with a target via the target recognition moiety, the photoactivatable molecule and the quencher molecule are separated. Thus, the spatial rearrangement of the photoactivatable molecule and quencher in the target-binding agent occurs only after interaction of the target recognition moiety with its target. Hence, the target recognition moiety is selected and positioned in the conjugate so that when the target recognition moiety interacts with its target, the spatial arrangement of the agent is changed such that the photoactivatable molecule is are no longer in a quenched state.

C. Conjugation of Target-binding Agents and/or Fusion Molecules to Red Blood Cells

In an embodiment, the target-binding agent may be bound to the surface of a modified red blood cell through a biotin-streptavidin bridge. For example, a biotinylated antibody may be linked to a non-specifically biotinylated cell surface through a streptavidin bridge. In an embodiment, the target-binding agent is attached to the red blood cell via the target recognition moiety, e.g., antibody. Antibodies can be conjugated to biotin by a number of chemical means (See, e.g., Hirsch et al., Methods Mol. Biol. 295: 135-154 (2004), which is incorporated herein by reference). The surface membrane proteins of a red blood cell may be biotinylated using an amine reactive biotinylation reagent such as, for example, EZ-Link Sulfo-NHS-SS-Biotin (sulfosuccinimidyl 2-(biotinamido)-ethyl-1,3-dithiopropionate; Pierce-Thermo Scientific, Rockford, Ill., USA; See, e.g., Jaiswal et al., Nature Biotech. 21:47-51 (2003), which is incorporated herein by reference). Isolated red blood cells may be incubated for 30 min at 4° C. in 1 mg/ml solution of sulfo-NHS-SS in phosphate-buffered saline. Excess biotin reagent is removed by washing the cells with Tris-buffered saline, for example. The biotinylated cells are then reacted with the biotinylated antibody in the presence of streptavidin to form the modified red blood cells.

In another embodiment, the target-binding agent may be attached to the surface of the modified red blood with a bispecific antibody, for example, with both target cell and red blood cell binding activities. The number of antigen binding sites on the modified red blood cell may range from about 0 to over 1000 sites, for example, depending upon the binding conditions (See, e.g., U.S. Pat. No. 5,470,570, which is incorporated herein by reference). The red blood cells may be further modified as described herein and re-introduced into an individual.

Alternatively, the bispecific antibody, for example, may be added directly to the bloodstream where it optimally binds in vivo to the modified red blood cell and to the target cell (See, e.g., U.S. Patent Application 2003/0215454, which is incorporated herein by reference). Alternatively, a unique receptor molecule may be expressed on the surface of a modified red blood cell that is detected by the bispecific antibody to ensure the selectivity of bispecific antibody to the modified red blood cell.

For example, the following receptors can be used to target macrophages: the complement receptor (Rieu et al., J Cell Biol. 127:2081-2091 (1994), which is incorporated herein by reference), the scavenger receptor (Brasseur et al., Photochem. Photobiol. 69:345-352 (1999), which is incorporated herein by reference), the transferrin receptor (Dreier et al., Bioconjug. Chem. 9:482-489 (1998); Hamblin et al., J. Photochem. Photobiol. 26:45-56 (1994)); the Fc receptor (Rojanasakul et al., Pharm. Res. 11:1731-1736 (1994)); the mannose receptor (Frankel et al., Carbohydr. Res. 300:251-258 (1997); Chakraborty et al., J. Protozool. 37:358-364 (1990), each of which is incorporated herein by reference). Target recognition moieties that can be conjugated with photoactivatable molecules, for example to target to macrophages, include low density lipoproteins (Mankertz et al., Biochem. Biophys. Res. Commun. 240:112-115 (1997); von Baeyer et al., Int. J. Clin. Pharmacol. Ther. Toxicol. 31:382-386 (1993)), very low density lipoproteins (Tabas et al., J. Cell Biol. 115:1547-1560 (1991)), mannose residues and other carbohydrate moieties (Pittet et al., Nucl. Med. Biol. 22:355-365 (1995)), poly-cationic molecules, such as poly-L-lysine (Hamblin et al., J. Photochem. Photobiol. 26:45-56 (1994)), liposomes (Bakker-Woudenberg et al., J. Drug Target. 2:363-371 (1994); Betageri et al., J. Pharm. Pharmacol. 45:48-53 (1993)), antibodies (Gruenheid et al., J. Exp. Med. 185:717-730, (1997)), and 2-macroglobulin (Chu et al., J. Immunol. 152:1538-1545 (1994), each of which is incorporated herein by reference).

In another embodiment, the target-binding agent is attached to the red blood cell via a covalent attachment. For example, the target recognition moiety may be derivatized and bound to the red blood cell using a coupling compound containing an electrophilic group that will react with nucleophiles on the red blood cell to form the interbonded relationship. Representative of these electrophilic groups are α,β unsaturated carbonyls, alkyl halides and thiol reagents such as substituted maleimides. In addition, the coupling compound can be coupled to the target recognition moiety via one or more of the functional groups in the target recognition moiety such as amino, carboxyl and tryosine groups. For this purpose, coupling compounds should contain free carboxyl groups, free amino groups, aromatic amino groups, and other groups capable of reaction with enzyme functional groups. Highly charged derivatives of target recognition moiety can also be prepared for immobilization on erythrocytes through electrostatic bonding. Examples of these derivatives would include polylysyl and polyglutamyl enzymes.

The choice of the reactive group embodied in the derivative depends on the reactive conditions employed to couple the electrophile with the nucleophilic groups on the red blood cell for immobilization. A controlling factor is the desire not to inactivate the coupling agent prior to coupling of the target recognition moiety immobilized by the attachment to the red blood cell.

Such coupling immobilization reactions can proceed in a number of ways. Typically, a coupling agent can be used to form a bridge between the macromolecule and the red blood cell. In this case, the coupling agent should possess a functional group such as a carboxyl group which can be caused to react with the target recognition moiety. One pathway for preparing the macromolecular derivative comprises the utilization of carboxyl groups in the coupling agent to form mixed anhydrides which react with the target recognition moiety, in which use is made of an activator which is capable of forming the mixed anhydride. Representative of such activators are isobutylchloroformate or other chi oroformates which give a mixed anhydride with coupling agents such as 5,5′-(dithiobis(2-nitrobenzoic acid) (DTNB), p-chloromercuribenzoate (CMB), or m-maleimidobenzoic acid (MBA). The mixed anhydride of the coupling agent reacts with the target recognition moiety to yield the reactive derivative which in turn can react with nucleophilic groups on the red blood cell to immobilize the macromolecule.

Functional groups on the target recognition moiety such as carboxyl groups can be activated with carbodiimides and the like activators. Subsequently, functional groups on the bridging reagent, such as amino groups, will react with the activated group on the target recognition moiety to form the reactive derivative. In addition, the coupling agent should possess a second reactive grouping which will react with appropriate nucleophilic groups on the red blood cell to form the bridge. Typical of such reactive groupings are alkylating agents such as iodoacetic acid, α,β unsaturated carbonyl compounds, such as acrylic acid and the like, thiol reagents, such as mercurials, substituted maleimides and the like.

Alternatively, functional groups on the target recognition moiety can be activated so as to react directly with nucleophiles on red blood cells to obviate the need for a bridge-forming compound. For this purpose, beneficial use is made of an activator such as Woodward's Reagent K or the like reagent which brings about the formation of carboxyl groups in the target recognition moiety into enol esters, as distinguished from mixed anhydrides. The enol ester derivatives of target recognition moieties will subsequently react with nucleophilic groups on the red blood cell to effect immobilization of the macromolecule.

D. Genetically Engineered Red Blood Cells

In an embodiment, red blood cell precursor cells are genetically engineered to express one or more protein- or RNA-based pharmaceuticals and/or one or more imaging agents (e.g., a fluorescent protein). This section describes the transformation of reticulocytes and hematopoietic stem cells, which are both precursor cells for mature erythrocytes.

1. Transformation of Reticulocytes

Isolated reticulocytes may be transfected with mRNA encoding proteins and/or peptides of interest. Messenger RNA may be derived from in vitro transcription of a cDNA plasmid construct containing the coding sequence corresponding to the protein and/or peptide of interest. For example, the cDNA sequence corresponding to the protein and/or peptide of interest may be inserted into a cloning vector containing promoter sequence compatible with specific RNA polymerases. For example, the cloning vector ZAP Express® pBK-CMV (Stratagene, La Jolla, Calif., USA) contains T3 and T7 promoter sequence compatible with T3 and T7 RNA polymerase, respectively. For in vitro transcription of sense mRNA, the plasmid is linearized at a restriction site downstream of the stop codon(s) corresponding to the end of the coding sequence of the protein and/or peptide of interest. The mRNA is transcribed from the linear DNA template using a commercially available kit such as, for example, the RNAMaxx® High Yield Transcription Kit (from Stratagene, La Jolla, Calif., USA). In some instances, it may be desirable to generate 5′-m⁷GpppG-capped mRNA. As such, transcription of a linearized cDNA template may be carried out using, for example, the mMES SAGE mMACHINE High Yield Capped RNA Transcription Kit from Ambion (Austin, Tex., USA). Transcription may be carried out in a reaction volume of 20-100 μl at 37° C. for 30 min to 4 h. The transcribed mRNA is purified from the reaction mix by a brief treatment with DNase Ito eliminate the linearized DNA template followed by precipitation in 70% ethanol in the presence of lithium chloride, sodium acetate or ammonium acetate. The integrity of the transcribed mRNA may be assessed using electrophoresis with an agarose-formaldehyde gel or commercially available Novex pre-cast TBE gels (e.g., Novex, Invitrogen, Carlsbad, Calif., USA).

Messenger RNA encoding proteins and/or peptides of interest may be introduced into reticulocytes using a variety of approaches including, for example, lipofection and electroporation (van Tandeloo et al., Blood 98:49-56 (2001), which is incorporated herein by reference). For lipofection, for example, 5 μg of in vitro transcribed mRNA in Opti-MEM (Invitrogen, Carlsbad, Calif., USA) is incubated for 5-15 min at a 1:4 ratio with the cationic lipid DMRIE-C (Invitrogen). Alternatively, a variety of other cationic lipids or cationic polymers may be used to transfect cells with mRNA including, for example, DOTAP, various forms of polyethylenimine, and polyL-lysine (Sigma-Aldrich, Saint Louis, Mo., USA), and Superfect (Qiagen, Inc., Valencia, Calif., USA; See, e.g., Bettinger et al., Nucleic Acids Res. 29:3882-3891 (2001), which is incorporated herein by reference). The resulting mRNA/lipid complexes are incubated with cells (1-2×10⁶ cells/ml) for 2 h at 37° C., washed and returned to culture. For electroporation, for example, about 5 to 20×10⁶ cells in 500 μl of Opti-MEM (Invitrogen, Carlsbad, Calif., USA) are mixed with about 20 μg of in vitro transcribed mRNA and electroporated in a 0.4-cm cuvette using, for example, and Easyject Plus device (EquiBio, Kent, United Kingdom). In some instances, it may be necessary to test various voltages, capacitances and electroporation volumes to determine the optimal conditions for transfection of a particular mRNA into a reticulocyte. In general, the electroporation parameters required to efficiently transfect cells with mRNA appear to be less detrimental to cells than those required for electroporation of DNA (van Tandeloo et al., Blood 98:49-56 (2001), which is incorporated herein by reference).

Alternatively, mRNA may be transfected into a reticulocyte using a peptide-mediated RNA delivery strategy (See, e.g., Bettinger et al., Nucleic Acids Res. 29:3882-3891 (2001), which is incorporated herein by reference). For example, the cationic lipid polyethylenimine 2 kDA (Sigma-Aldrich, Saint Louis, Mo., USA) may be combined with the melittin peptide (Alta Biosciences, Birmingham, UK) to increase the efficiency of mRNA transfection, particularly in post-mitotic primary cells. The mellitin peptide may be conjugated to the PEI using a disulfide cross-linker such as, for example, the hetero-bifunctional cross-linker succinimidyl 3-(2-pyridyldithio)propionate. In vitro transcribed mRNA is preincubated for 5 to 15 min with the mellitin-PEI to form an RNA/peptide/lipid complex. This complex is then added to cells in serum-free culture medium for 2 to 4 h at 37° C. in a 5% CO₂ humidified environment and then removed and the transfected cells allowed to continue growing in culture.

2. Transformation of Hematopoetic Stem Cells

Non-endogenous proteins such as, for example, receptors, enzymes and/or therapeutic peptides may be genetically introduced into hematopoietic stem cells prior to terminal differentiation using a variety of DNA techniques, including transient or stable transfections and gene therapy approaches. These non-endogenous proteins expressed on the surface and/or in the cytoplasm of mature red blood cell may be used to target the modified red blood cell to a specific location, to bind specific blood analytes, to react and/or signal in the presence of specific analytes, and/or to treat a specific disease or condition.

Viral based gene transfer. Viral gene transfer may be used to transfect hematopoietic stem cells with DNA encoding proteins and/or peptides of interest (Papapetrou et al., Gene Therapy 12:S118-S130 (2005), which is incorporated herein by reference). A number of viruses may be used as gene transfer vehicles including Moloney murine leukemia virus (MMLV), adenovirus, adeno-associated virus, herpes simplex virus (HSV), lentiviruses such as human immunodeficiency virus 1 (HIV 1), and spumaviruses such as foamy viruses, for example (See, e.g., Osten et al., HEP 178:177-202 (2007), which is incorporated herein by reference). Retroviruses, for example, efficiently transduce mammalian cells including human cells and integrate into chromosomes, conferring stable gene transfer.

A cell membrane associated receptor, for example, may be transcribed into hematopoietic stem cells and subsequently expressed in a mature red blood cell using a Moloney murine leukemia virus (MMLV) vector backbone (Malik et al., Blood 91:2664-2671 (1998), which is incorporated herein by reference). Vectors based on MMLV, an oncogenic retrovirus, are currently used in gene therapy clinical trials (Hossle et al., News Physiol. Sci. 17:87-92 (2002), which is incorporated herein by reference). A DNA construct containing the cDNA encoding a cell membrane associated receptor such as, for example, the mu opioid receptor is generated in the MMLV vector backbone using standard molecular biology techniques. The construct is transfected into a packaging cell line such as, for example, PA317 cells and the viral supernatant is used to transfect producer cells such as, for example, PG13 cells. The PG13 viral supernatant is incubated with hematopoietic stem cells that have been isolated and cultured as described in above. The expression of the cell membrane associated receptor such as, for example, the mu opioid receptor may be monitored using FACS analysis (fluorescence-activated cell sorting), for example, with a fluorescently labeled antibody directed against the cell membrane associated receptor. Similar methods may be used to express a cytoplasmic protein such as, for example, a modified hemoglobin molecule (See, e.g., Nicolini et al., Blood 100:1257-1264 (2002), which is incorporated herein by reference) or a small peptide such as, for example, a cytokine (See, e.g., Song et al., Cancer Res. 66:6304-6311 (2006), which is incorporated herein by reference) in a hematopoietic stem cell.

Similarly, a fluorescent tracking molecule.such as, for example, green fluorescent protein (GFP) may be transfected into hematopoietic stem cells using a viral-based approach (Tao et al., Stem Cells 25:670-678 (2007), which is incorporated herein by reference). As such, bone marrow cells are isolated and cultured as described herein. Two days prior to transfection, the cells are prestimulated in minimum essential medium (MEM) containing 20% fetal bovine serum, 4 mM L-glutamine, 100 units/ml penicillin, 100 μg/m1 streptomycin, 100 ng/ml murine stem cell factor, 100 ng/ml murine FLT3-ligand, and 100 ng/ml murine thrombopoietin. Ecotopic retroviral vectors containing DNA encoding the enhanced green fluorescent protein (EGFP) or a red fluorescent protein (e.g., DsRed-Express) are packaged using a packaging cell such as, for example, the Phoenix-Eco cell line (distributed by Orbigen, San Diego, Calif.). Packaging cell lines stably express viral proteins needed for proper viral packaging including, for example, gag, pol, and env. Supernatants from the Phoenix-Eco cells into which viral particles have been shed are used to transduce prestimulated hematopoietic stem cells. hi some instances, transduction may be performed on a specially coated surface such as, for example, fragments of recombinant fibronectin to improve the efficiency of retroviral mediated gene transfer (e.g., RetroNectin, Takara Bio USA, Madison, Wis.). As such, prestimulated cells are incubated in RetroNectin-coated plates with retroviral Phoenix-Eco supernatants plus 100 ng/ml murine stem cell factor, 100 ng/ml murine FLT3-ligand, and 100 ng/ml murine thrombopoietin. After incubation at 37° C., plates are centrifuged at 400×g for 5 min at 20° C. and further incubated at 37° C. for 5.5 h. Transduction may be repeated the next day. In this instance, the percentage of cells expressing EGFP or DsRed-Express may be assessed by FACS. Other reporter genes that may be used to assess transduction efficiency include, for example, beta-galactosidase, chloramphenicol acetyltransferase, and luciferase as well as low-affinity nerve growth factor receptor (LNGFR), and the human cell surface CD24 antigen (Bierhuizen et al., Leukemia 13:605-613 (1999), which is incorporated herein by reference).

Non-viral gene transfer. Nonviral vectors may be used to introduce genetic material into hematopoietic stem cells (Papapetrou et al., Gene Therapy 12:S118-S130 (2005), which is incorporated herein by reference). Nonviral-mediated gene transfer differs from viral-mediated gene transfer in that the plasmid vectors contain no proteins, are less toxic and easier to scale up, and have no host cell preferences. The “naked DNA” of plasmid vectors are by themselves inefficient in delivering genetic material to a cell and therefore are combined with a gene delivery method that enables entry into cells. A number of delivery methods may be used to transfer nonviral vectors into hematopoietic stem cells including chemical and physical methods.

A nonviral vector encoding a protein and/or peptide of interest may be introduced into hematopoietic stem cells using synthetic macromolecules such as cationic lipids and polymers (Papapetrou et al., Gene Therapy 12:S118-S130 (2005), which is incorporated herein by reference). Cationic liposomes, for example, form complexes with DNA through charge interactions. The positively charged DNA/lipid complexes bind to the negative cell surface and are taken up by the cell by endocytosis. This approach may be used, for example, to transfect hematopoietic cells (See, e.g., Keller et al., Gene Therapy 6:931-938 (1999), which is incorporated herein by reference). In an embodiment, the liposome includes at least one natural phospholipid. Liposomes including natural phospholipids are generally biologically inert, and have low intrinsic toxicity. See, for example, Immordino, et al., Int. J. Nanomed. vol 1(3): 297-315 (2006), which is incorporated herein by reference.

Hematopoietic cells are cultured in association with adherent stromal cells as described herein. The plasmid DNA (approximately 0.5 μg in 25-100 μL of a serum free medium, such as, for example, OptiMEM (Invitrogen, Carlsbad, Calif.)) is mixed with a cationic liposome (approximately 4 μg in 25 μL of serum free medium) such as the commercially available transfection reagent Lipofectamine™ (Invitrogen, Carlsbad, Calif.) and allowed to incubate for at least 20 min to form complexes. The DNA/liposome complex is added to the hematopoietic cells and allowed to incubate for 5-24 h, after which time transgene expression may be assayed. Alternatively, other commercially available liposome tranfection agents may be used (e.g., In vivo GeneSHUTTLE™, Qbiogene, Carlsbad, Calif.).

Alternatively, a cationic polymer such as, for example, polyethylenimine (PEI) may be used to efficiently transfect hematopoietic and umbilical cord blood-derived CD34+ cells (See, e.g., Shin et al., Biochim. Biophys. Acta 1725:377-384 (2005), which is incorporated herein by reference). Human CD34+ cells are isolated from human umbilical cord blood as described herein and cultured in Iscove's modified Dulbecco's medium supplemented with 200 ng/ml stem cell factor and 20% heat-inactivated fetal bovine serum. Plasmid DNA encoding the protein or proteins of interest is incubated with branched or linear PEIs varying in size from 0.8 K to 750 K (Sigma Aldrich, Saint Louis, Mo., USA; Fermetas, Hanover, Md., USA). PEI is prepared as a stock solution at 4.2 mg/ml distilled water and slightly acidified to pH 5.0 using HCl. The DNA may be combined with the PEI for 30 min at room temperature at various nitrogen/phosphate ratios based on the calculation that 1 μg of DNA contains 3 nmol phosphate and 1 μl of PEI stock solution contains. 10 nmol amine nitrogen. The isolated CD34+ cells are seeded with the DNA/cationic complex, centrifuged at 280×g for 5 min and incubated in culture medium for 4 or more h until gene expression is assessed.

A plasmid vector may be introduced into a hematopoietic stem cell using a physical method such as particle-mediated transfection, “gene gun”, biolistics, or particle bombardment technology (Papapetrou, et al, (2005) Gene Therapy 12:S118-S130). In this instance, DNA encoding the protein and/or peptides of interest is absorbed onto gold particles and administered to cells by a particle gun. This approach may be used, for example, to transfect hematopoietic stem cells derived from umbilical cord blood (See, e.g., Verma et al., Gene Therapy 5:692-699 (1998), which is incorporated herein by reference). As such, umbilical cord blood is isolated and diluted three fold in phosphate buffered saline. CD34+ cells are purified using an anti-CD34 monoclonal antibody in combination with magnetic microbeads coated with a secondary antibody and a magnetic isolation system (e.g., Miltenyi MiniMac System, Auburn, Calif., USA). The CD34+ enriched cells may be cultured as described herein. Alternatively, the CD34+ enriched cells may be cultured on irradiated stromal cells in IMDM medium, for example, with 20% fetal bovine serum, 1% deionized bovine serum albumin, penicillin/streptomycin, L-glutamine, 2-mercaptoethanol and hydrocortisone supplemented with IL-3 (5 ng/ml), IL-6 (25 ng/ml), and stem cell factor (50 ng/ml). For transfection, plasmid DNA is precipitated onto a particle, for example gold beads, by treatment with calcium chloride and spermidine. Following washing of the DNA-coated beads with ethanol, the beads may be delivered into the cultured cells using, for example, a Biolistic PDS-1000/He System (Bio-Rad, Hercules, Calif., USA). A reporter gene such as, for example, beta-galactosidase, chloramphenicol acetyltransferase, luciferase, or green fluorescent protein may be used to assess efficiency of transfection.

Alternatively, electroporation methods may be used to introduce a plasmid vector into hematopoietic stem cells (See, e.g., Wu et al., Gene Ther. 8:384-390 (2001), which is incorporated herein by reference). Electroporation creates transient pores in the cell membrane, allowing for the introduction of various molecules into the cells including, for example, DNA and RNA as well as antibodies and drugs. As such, CD34+ cells are isolated and cultured as described herein. Immediately prior to electroporation, the cells are isolated by centrifugation for 10 min at 250×g at room temperature and resuspended at 0.2-10×10⁶ viable cells/ml in an electroporation buffer such as, for example, X-VIVO 10 supplemented with 1.0% human serum albumin (HSA). The plasmid DNA (1-50 μg) is added to an appropriate electroporation cuvette along with 500 μl of cell suspension. Electroporation may be done using, for example, an ECM 600 electroporator (Genetronics, San Diego, Calif., USA) with voltages ranging from 200 V to 280 V and pulse lengths ranging from 25 to 70 milliseconds. A number of alternative electroporation instruments are commercially available and may be used for this purpose (e.g., Gene Pulser Xcell™, BioRad, Hercules, CA; Cellject Duo, Thermo Science, Milford, Mass.). Alternatively, efficient electroporation of isolated CD34+ cells may be performed using the following parameters: 4 mm cuvette, 1600 μF, 550 V/cm, and 10 μg of DNA per 500 μl of cells at 1×10⁵ cells/ml (Oldak et al., Acta Biochimica Polonica 49:625-632 (2002), which is incorporated herein by reference).

Nucleofection, a form of electroporation, may also be used to transfect hematopoietic stem cells, or other cells. In this instance, transfection is performed using electrical parameters in cell-type specific solutions that enable DNA (or other reagents) to be directly transported to the nucleus thus reducing the risk of possible degradation in the cytoplasm. For example, a Human CD34 Cell Nucleofector™ Kit (from amaxa inc.) may be used to transfect hematopoietic stem cells. In this instance, 1-5×10⁶ cells in Human CD34 Cell Nucleofector™ Solution are mixed with 1-5 μg of DNA and transfected in the Nucleofector™ instrument using preprogrammed settings as determined by the manufacturer.

Hematopoietic stem cells, or other cells, may be non-virally transfected with a conventional expression vector which is unable to self-replicate in mammalian cells unless it is integrated in the genome. Alternatively, hematopoietic stem cells may be transfected with an episomal vector which may persist in the host nucleus as autonomously replicating genetic units without integration into chromosomes (Papapetrou et al., Gene Therapy 12:S118-S130 (2005), which is incorporated herein by reference). These vectors exploit genetic elements derived from viruses that are normally extrachromosomally replicating in cells upon latent infection such as, for example, EBV, human polyomavirus BK, bovine papilloma virus-1 (BPV-1), herpes simplex virus-1 (HSV) and Simian virus 40 (SV40). Mammalian artificial chromosomes may also be used for nonviral gene transfer (Vanderbyl et al., Exp. Hematol. 33:1470-1476 (2005), which is incorporated herein by reference).

In an embodiment, the artificial antigen presenting cell includes at least one switchable surface. For example, the switchable surface includes materials (such as MHC-epitope complexes) that are configured to be transformed from a first state to a second state. In an embodiment, the switchable surface includes altering a conformation state from a cis to a trans configured double bond, rotating a molecular group about an axis, opening a hinged molecular group, bending a molecular chain, or unbending a molecular chain. See, for example, U.S. Pat. App. Pub. No. 20060263033, which is incorporated herein by reference. In an embodiment, the surface is configured to be switchable by at least one of applying a voltage, voltage change, temperature change, pH change, exposure to LTV light, exposure to electromagnetic radiation, exposure to magnetic field, removal of magnetic field, change in capacitance, exposure to electrostatic charge, or removal of electrostatic charge. In an embodiment, the switchable surface is reversible.

In an embodiment, the at least one artificial antigen presenting cell complex is bound to the lipid bilayer, polymeric vehicle, cell, or other artificial antigen presenting cell vehicle. In an embodiment, the at least one artificial antigen presenting cell complex is unbound, or free floating in the surface of the vehicle.

In an embodiment, the artificial antigen presenting cell complex includes at least one MHC:epitope, optionally including at least one immunomodulatory molecule. In an embodiment, the immunomodulatory molecule includes at least a portion of one or more of a co-stimulatory molecule, accessory molecule, adhesion molecule, cytokine, cytokine receptor, chemokine, chemokine receptor, anergy-inducing molecule, cell death-inducing molecule, or differentiation-inducing molecule. In an embodiment, including at least one immunomodulator molecule improves the ability of the artificial antigen presenting cell to modulate an immune response. See, for example, U.S. Patent App. Pub. No. 20050208120 A1, which is incorporated herein by reference.

III. Loading of a Target-Binding Agent or Molecular Agent into Red Blood Cells

In an embodiment, red blood cells are loaded with one or more target-binding agents, such that the one or more target-binding agents are internalized within the red blood cell. In an embodiment, red blood cells are loaded with one or more molecular agents. A molecular agent may include, but is not limited to, a compound that is configured to provide an activity to the subject and/or to the red blood cell following administration. In an embodiment, such agent may include, but is not limited to, one or more therapeutic agents or imaging agents.

In an embodiment, a target-binding agent or molecular agent includes an artificial antigen presenting cell complex.

A. Methods of Loading Red Blood Cells

A number of methods may be used to load modified red blood cells with an agent (e.g., target-binding agent or molecular agent) such as, for example, hypotonic lysis, hypotonic dialysis, osmosis, osmotic pulsing, osmotic shock, ionophoresis, electroporation, sonication, microinjection, calcium precipitation, membrane intercalation, lipid mediated transfection, detergent treatment, viral infection, diffusion, receptor mediated endocytosis, use of protein transduction domains, particle firing, membrane fusion, freeze-thawing, mechanical disruption, and filtration (See, e.g., U.S. Pat. No. 6,495,351 B2; U.S. Patent Application 2007/0243137 A1, each of which is incorporated herein by reference).

For hypotonic lysis, modified red blood cells are exposed to low ionic strength buffer causing them to burst. The therapeutic agent such as an antibiotic or chemotherapeutic agent, for example, distributes within the cells. Red blood cells may be hypotonically lysed by adding 30-50 fold volume excess of 5 mM phosphate buffer (pH 8), for example, to a pellet of isolated red blood cells. The resulting lysed cell membranes are isolated by centrifugation. The pellet of lysed red blood cell membranes is resuspended and incubated in the presence of the therapeutic agent in a low ionic strength buffer for 30 min, for example. Alternatively, the lysed red blood cell membranes may be incubated with the therapeutic agent for as little as one minute or as long as several days, depending upon the best conditions determined to efficiently load the cells.

Alternatively, red blood cells may be loaded with a therapeutic agent using controlled dialysis against a hypotonic solution to swell the cells and create pores in the cell membrane (See, e.g., U.S. Pat. Nos. 4,327,710, 5,753,221, and 6,495,351 B2, each of which is incorporated herein by reference). For example, a pellet of isolated red blood cells is resuspended in 10 mM HEPES, 140 mM NaCl, 5 mM glucose pH 7.4 and dialyzed against a low ionic strength buffer containing 10 mM NaH₂PO₄, 10 mM NaHCO₃, 20 mM glucose, and 4 mM MgCl₂, pH 7.4. After 30-60 min, the red blood cells are further dialyzed against 16 mM NaH₂PO₄, pH 7.4 solution containing the therapeutic agent for an additional 30-60 min. All of these procedures may be optimally performed at a temperature of 4° C. In some instances, it may be beneficial to load a large quantity of red blood cells with a therapeutic agent by a dialysis approach and as such a specific apparatus designed for this purpose may be used (See, e.g., U.S. Pat. Nos. 4,327,710, 6,139,836 and 6,495,351 B2, each of which is incorporated herein by reference).

The loaded red blood cells can be resealed by gentle heating in the presence of a physiological solution such as, for example, 0.9% saline, phosphate buffered saline, Ringer's solution, cell culture medium, blood plasma or lymphatic fluid. For example, well-sealed membranes may be generated by treating the disrupted red blood cells for 1-2 min in 150 mM salt solution of, for example, 100 mM phosphate (pH 8.0) and 150 mM sodium chloride at a temperature of 60° C. Alternatively, the cells may be incubated at a temperature of 25-50° C. for 30 min to 4 h, for example (See, e.g., U.S. Patent Application 2007/0243137 A1, which is incorporated herein by reference). Alternatively, the disrupted red blood cells may be resealed by incubation in 5 mM adenine, 100 mM inosine, 2 mM ATP, 100 mM glucose, 100 mM Na-pyruvate, 4 mM MgCl₂, 194 mM NaCl, 1.6 M KCl, and 35 mM NaH₂PO₄, pH 7.4 at a temperature of 37° C. for 20-30 min (See, e.g., U.S. Pat. No. 5,753,221, which is incorporated herein by reference).

For electroporation, for example, modified red blood cells are exposed to an electrical field which causes transient holes in the cell membrane, allowing the therapeutic agent to diffuse into the cell (See, e.g., U.S. Pat. No. 4,935,223, which is incorporated herein by reference). Modified red blood cell are suspended in a physiological and electrically conductive media such as, for example, platelet-free plasma to which the therapeutic agent is added. The mixture in a volume ranging from 0.2 to 1.0 ml is placed in an electroporation cuvette and cooled on ice for 10 min. The cuvette is placed in an electroporation apparatus such as, for example, an ECM 830 (from BTX Instrument Division, Harvard Apparatus, Holliston, Mass.). The cells are electroporated with a single pulse of approximately 2.4 milliseconds in length and a field strength of approximately 2.0 kV/cm. Alternatively, electroporation of red blood cells may be carried out using double pulses of 2.2 kV delivered at 0.25 μF using a Bio-Rad Gene Pulsar apparatus (Bio-Rad, Hercules, Calif., USA) to achieve a loading capacity of over 60% (Flynn et al., Cancer Lett. 82:225-229 (1994), which is incorporated herein by reference). The cuvette is returned to the ice bath for 10-60 min and then placed in a 37° C. water bath to induce resealing of the cell membrane.

For sonication, for example, modified red blood cells are exposed to high intensity sound waves, causing transient disruption of the cell membrane allowing the therapeutic agent to diffuse into the cell.

For detergent treatment, for example, modified red blood cells are treated with a mild detergent which transiently compromises the cell membrane by creating holes, for example, through which the therapeutic agent may diffuse. After cells are loaded, the detergent is washed from the cells. For example, the detergent may be saponin. For receptor mediated endocytosis, the modified red blood cell may have a surface receptor which upon binding of the therapeutic agents induce internalization of the receptor and the associated therapeutic agent.

In an embodiment, the therapeutic agent may be loaded into a modified red blood cell by fusing or conjugating the agent to proteins and/or peptides capable of crossing or translocating the plasma membrane (See, e.g., U.S. Patent Application 2002/0151004 A1, which is incorporated herein by reference). Examples of protein domains and sequences that are capable of translocating a cell membrane include, for example, sequences from the HIV-1-transactivating protein (TAT), the Drosophila Antennapedia homeodomain protein, the herpes simplex-1 virus VP22 protein, and transportin, a fusion between the neuropeptide galanin and the wasp venom peptide mastoparan. As such, a therapeutic agent may be fused or conjugated to all or part of the TAT peptide, for example. A fusion protein containing all or part of the TAT peptide and the therapeutic agent such as an antibody, enzyme, or peptide, for example, may be generated using standard recombinant DNA methods. Alternatively, all or part of the TAT peptide may be chemically coupled to a functional group associated with the therapeutic agent such as, for example, a hydroxyl, carboxyl or amino group. In some instances, the link between the TAT peptide and the therapeutic agent may be pH sensitive such that once the complex has entered the intracellular environment, the therapeutic agent is separated from the TAT peptide.

For mechanical firing, for example, the modified red blood cell may be bombarded with the therapeutic agent attached to a heavy or charged particle such as, for example, gold microcarriers and are mechanically or electrically accelerated such that they traverse the cell membrane. Microparticle bombardment of this sort may be achieved using, for example, the Helios Gene Gun (from, e.g., Bio-Rad, Hercules, Calif., USA).

Alternatively, the modified red blood cell may be loaded with a therapeutic agent by fusion with a synthetic vesicle such as, for example, a liposome. In this instance, the vesicles themselves are loaded with the therapeutic agent using one or more of the methods described herein. Alternatively, the therapeutic agent may be loaded into the vesicles during vesicle formation. The loaded vesicles are then fused with the modified red blood cells under conditions that enhance cell fusion. Fusion of a liposome, for example, with a cell may be facilitated using various inducing agents such as, for example, proteins, peptides, polyethylene glycol (PEG), and viral envelope proteins or by changes in medium conditions such as pH (See, e.g., U.S. Pat. No. 5,677,176, which is incorporated herein by reference).

In an embodiment, the liposome utilized herein includes at least one of phosphatidylcholine, cholesterol, phosphatidylethanolamine, or other phospholipid. In an embodiment, the liposome utilized herein includes at least one of dioleoylphosphatidylethanolamine, or other surfactant. In an embodiment, the liposome utilized herein includes at least one ganglioside.

For filtration, the modified red blood cell and the therapeutic agent may be forced through a filter of pore size smaller than the red blood cell causing transient disruption of the cell membrane and allowing the therapeutic agent to enter the cell. For freeze thawing, the modified red blood cells are sent through several freeze thaw cycles, resulting in cell membrane disruption (See, e.g., U.S. Patent Application 2007/0243137 A1, which is incorporated herein by reference). In this instance, a pellet of packed red blood cells (0.1-1.0 ml) is mixed with an equal volume (0.1-1.0 ml) of an isotonic solution (e.g., phosphate buffered saline) containing the therapeutic agent. The red blood cells are frozen by immersing the tube containing the cells and therapeutic agent into liquid nitrogen, for example. Alternatively, the cells may be frozen by placing the tube in a freezer at −20° C. or −80° C., for example. The cells are then thawed in a 23° C. water bath and the cycle repeated if necessary to increase loading.

The therapeutic agent may be selected from a variety of known small molecule pharmaceuticals. Alternatively, the therapeutic agent may be an inactivating peptide nuclei acid (PNA), an RNA or DNA oligonucleotide aptamer, an interfering RNA (iRNA), a peptide, or a protein. The therapeutic agent may be loaded into the cell in a solubilized form. As such, the therapeutic agent is solubilized in an appropriate buffer prior to loading into red blood cells.

Alternatively, the therapeutic agent may be loaded into red blood cells in a particulate form as a solid microparticulate (See, e.g., U.S. Patent Applications 2005/0276861 A1 and U.S. 2006/0270030 A1, each of which is incorporated herein by reference). In this instance, the therapeutic agent may be poorly water-soluble with a solubility of less than 1-10 mg/ml. As such, microparticles of less than 10 μm may be generated using a variety of techniques such as, for example, energy addition techniques such as milling (e.g., pearl milling, ball milling, hammer milling, fluid energy milling, jet milling), wet grinding, cavitation or shearing with a microfluidizer, and sonication; precipitation techniques such as, for example, microprecipitation, emulsion precipitation, solvent-antisolvent precipitation, phase inversion precipitation, pH shift precipitation, infusion precipitation, temperature shift precipitation, solvent evaporation precipitation, reaction precipitation, compressed fluid precipitation, protein microsphere precipitation; and other techniques such as spraying into cryogenic fluids (See, e.g., U.S. Patent Application 2005/0276861 A1, which is incorporated herein by reference). Water soluble molecules may also be used to form solid microparticles in the presence of various polymers such as, for example, polylactate- polyglycolate copolymer (PLGA), polycyanoacrylate, albumin, and/or starch (See, e.g., U.S. Patent Application 2005/0276861 A1, which is incorporated herein by reference). Alternatively, a water soluble molecule may be encapsulated in a vesicle to form a microparticle. The microparticles composed of the therapeutic agent may be incorporated into a modified red blood cell using the methods described herein.

A modified red blood cell loaded with a therapeutic agent may be administered intravenously, intramuscularly, subcutaneously, intradermally, intra-articularly, intrathecally, epidurally, intracerebrally, by buccal administration, rectally, topically, transdermally, orally, intranassaly, by pulmonary route, intraperitoneally, intra-opthalmically, or retro-orbitally. The cells may be administered by bolus injection, by intermittent infusion, or by continuous infusion, for example.

B. Molecular Agents

A variety of different agents may be loaded into red blood cells as described above. It will be appreciated that it is not necessary for a single agent to be used, and that it is possible to load two or more agents into a cell. Accordingly, the term “agent” also includes mixtures, fusions, combinations and conjugates, of atoms, molecules, etc. as disclosed herein. For example, an agent may include, but is not limited to, a nucleic acid combined with a polypeptide; two or more polypeptides conjugated to each other; a protein conjugated to a biologically active molecule (which may be a small molecule such as a prodrug); or a combination of a biologically active molecule with an imaging agent.

1. Therapeutic Agents

In an embodiment, the molecular agent is a therapeutic agent, such as a small molecule drug or biological effector molecule. For example, the therapeutic agent may be a biological effector molecule which has activity in a biological system. Biological effector molecules, include, but are not limited to, a protein, polypeptide, or peptide, including, but not limited to, a structural protein, an enzyme, a cytokine (such as an interferon and/or an interleukin), a polyclonal or monoclonal antibody, or an effective part thereof, such as an Fv fragment, which antibody or part thereof, may be natural, synthetic or humanized, a peptide hormone, a receptor, or a signaling molecule. Included within the term “immunoglobulin” are intact immunoglobulins as well as antibody fragments such as Fv, a single chain Fv (scFv), a Fab or a F(ab′)₂. Therapeutic agents of interest include, without limitation, pharmacologically active drugs, genetically active molecules, etc. Therapeutic agents of interest include antineoplastic agents, anti-inflammatory agents, hormones or hormone antagonists, ion channel modifiers, and neuroactive agents. Examples of of therapeutic agents include those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (ed.), Chemical Warfare Agents, Academic Press, New York (1992), which is incorporated herein by reference).

In an embodiment, the biological effector molecules are immunoglobulins, antibodies, Fv fragments, etc., that are capable of binding to antigens in an intracellular environment. These types of molecules are known as “intrabodies” or “intracellular antibodies.” An “intracellular antibody” or an “intrabody” includes an antibody that is capable of binding to its target or cognate antigen within the environment of a cell, or in an environment that mimics an environment within the cell. Selection methods for directly identifying such “intrabodies” include the use of an in vivo two-hybrid system for selecting antibodies with the ability to bind to antigens inside mammalian cells. Such methods are described in PCT/GB00/00876, incorporated herein by reference. Techniques for producing intracellular antibodies, such as anti-β-galactosidase scFvs, have also been described in Martineau et al., J. Mol Biol 280:117-127 (1998) and Visintin et al., Proc. Natl. Acad. Sci. USA 96:11723-1728 (1999); each of which is incorporated herein by reference.

In an embodiment, the biological effector molecule includes, but is not limited to, at least one of a protein, a polypeptide, a peptide, a nucleic acid, a virus, a virus-like an amino acid, an amino acid analogue, a modified amino acid, a modified amino acid analogue, a steroid, a proteoglycan, a lipid and a carbohydrate or a combination thereof (e.g., chromosomal material comprising both protein and DNA components or a pair or set of effectors, wherein one or more convert another to active form, for example catalytically).

A biological effector molecule may include a nucleic acid, including, but not limited to, an oligonucleotide or modified oligonucleotide, an antisense oligonucleotide or modified antisense oligonucleotide, an aptamer, a cDNA, genomic DNA, an artificial or natural chromosome (e.g., a yeast artificial chromosome) or a part thereof, RNA, including an siRNA, a shRNA, mRNA, tRNA, rRNA or a ribozyme, or a peptide nucleic acid (PNA); a virus or virus-like particles; a nucleotide or ribonucleotide or synthetic analogue thereof, which may be modified or unmodified.

The biological effector molecule can also be an amino acid or analogue thereof, which may be modified or unmodified or a non-peptide (e.g., steroid) hormone; a proteoglycan; a lipid; or a carbohydrate. If the biological effector molecule is a polypeptide, it can be loaded directly into a modified red blood cell, according to the methods described herein. Alternatively, a nucleic acid molecule bearing a sequence encoding a polypeptide, which sequence is operatively linked to transcriptional and translational regulatory elements active in a cell at a target site, may be loaded.

Small molecules, including inorganic and organic chemicals, may also be used. In an embodiment, the small molecule is a pharmaceutically active agent. Useful classes of pharmaceutically active agents include, but are not limited to, antibiotics, anti-inflammatory drugs, angiogenic or vasoactive agents, growth factors and chemotherapeutic (anti-neoplastic) agents (e.g., tumour suppressers). If a prodrug is loaded in an inactive form, a second effector molecule may be loaded into a modified red blood cell, or a red blood cell that is to be modified according to the disclosure herein. Such a second effector molecule is usefully an activating polypeptide which converts the inactive prodrug to active drug form. In an embodiment, activating polypeptides include, but are not limited to, viral thymidine kinase (encoded by Genbank Accession No. J02224), carboxypeptidase A (encoded by Genbank Accession No. M27717), α-galactosidase (encoded by Genbank Accession No. M13571), β-gluucuronidase (encoded by Genbank Accession No. M15182), alkaline phosphatase (encoded by Genbank Accession No. J03252 J03512), or cytochrome P-450 (encoded by Genbank Accession No. D00003 N00003), plasmin, carboxypeptidase G2, cytosine deaminase, glucose oxidase, xanthine oxidase, β-glucosidase, azoreductase, t-gutamyl transferase, β-lactamase, or penicillin amidase.

Either the polypeptide or the gene encoding it may be loaded into the modified, or to-be-modified, red blood cells; if the latter, both the prodrug and the activating polypeptide may be encoded by genes on the same recombinant nucleic acid construct. Furthermore, either the prodrug or the activator of the prodrug may be transgenically expressed in hematopoietic stem cells and already loaded into the red blood cell. The relevant activator or prodrug (as the case may be) is then loaded as a second agent according to the methods described herein.

2. Imaging Agents

The agent may be an imaging agent, by which term is meant an agent which may be detected, whether in vitro or in vivo in the context of a tissue, organ or organism in which the agent is located. Examples of agents include those useful for imaging of tissues in vivo or ex vivo. For example, imaging agents, such as labeled antibodies which are specific for defined molecules, tissues or cells in an organism, may be used to image specific parts of the body by releasing from the loaded red blood cells at a desired location using electromagnetic radiation. This allows imaging agents which are not completely specific for the desired target, and which might otherwise lead to more general imaging throughout the organism, to be used to image defined tissues or structures. For example, in an embodiment, an antibody which is capable of imaging endothelial tissue is used to image endothelial cells in lower body vasculature, such as in the lower limbs, by releasing the antibody selectively in the lower body by applying ultrasound thereto. The electromagnetic energy will preferentially lyse the red blood cells in the desired target site, thereby achieving selective therapeutic effects with minimal damage to normal cells.

In an embodiment, the imaging agent emits a detectable signal, such as visible light or other electromagnetic radiation. In another embodiment, the imaging agent is a radioisotope, for example ³²P or ³⁵S or ⁹⁹Tc, or a quantum dot, or a molecule such as a nucleic acid, polypeptide, or other molecule, conjugated with such a radioisotope. In an embodiment, the imaging agent is opaque to radiation, such as X-ray radiation. In another embodiment, the imaging agent comprises a targeting functionality by which it is directed to a particular cell, tissue, organ or other compartment within the body of an animal. For example, the agent may comprise a radiolabelled antibody which specifically binds to defined molecule(s), tissue(s) or cell(s) in an organism.

In an embodiment, the imaging agent is a contrast dye. For example, an MRI contrast agent can comprise a paramagnetic contrast agent (such as a gadolinium compound), a superparamagnetic contrast agent (such as iron oxide nanoparticles), a diamagnetic agent (such as barium sulfate), and combinations thereof. Metal ions preferred for MRI include those with atomic numbers 21-29, 39-47, or 57-83, and, more typically, a paramagnetic form of a metal ion with atomic numbers 21-29, 42, 44, or 57-83. Particularly preferred paramagnetic metal ions are selected from the group consisting of Gd(III), Fe(III), Mn(II and III), Cr(III), Cu(II), Dy(III), Tb(III and IV), Ho(III), Er(III), Pr(III) and Eu(II and III). Gd(III) is particularly useful. Note that as used herein, the term “Gd” is meant to convey the ionic form of the metal gadolinium; such an ionic form can be written as GD(III), GD3+, etc. with no difference in ionic form contemplated. A CT contrast agent can comprise iodine (ionic or non-ionic formulations), barium, barium sulfate, Gastrografin (a diatrizoate meglumine and diatrizoate sodium solution), and combinations thereof. In another embodiment, a PET or SPECT contrast agent can comprise a metal chelate.

IV. Incorporating Positive Marker(s) into Modified Red Blood Cells

The modified red blood cells may also be labeled with one or more positive markers that can be used to monitor over time the number or concentration of modified red blood cells in the blood circulation of an individual. It is anticipated that the overall number of modified red blood cells will decay over time following initial transfusion. As such, it may be appropriate to correlate the signal from one or more positive markers with that of the activated molecular marker, generating a proportionality of signal that will be independent of the number of modified red blood cells remaining in the circulation. There are presently several fluorescent compounds, for example, that are approved by the Food & Drug Administration for human use including but not limited to fluorescein, indocyanin green, and rhodamine B. For example, red blood cells may be non-specifically labeled with fluorescein isothiocyanate (FITC; Bratosin et al., Cytometry 46:351-356 (2001), which is incorporated herein by reference). A solution of FITC-labeled lectins in phosphate buffered saline (PBS) with 0.2 mM phenylmethysulfonyl fluoride (PMSF) is added to an equal volume of isolated red blood cells in the same buffer. The cells are incubated with the FITC-labeled lectins for 1 h at 4° C. in the dark. The lectins bind to sialic acids and beta-galactosyl residues on the surface of the red blood cells.

It is anticipated that other dyes may be useful for tracking modified red blood cells in human and non-human circulation. A number of reagents may be used to non-specifically label a red blood cell. For example red blood cells may be labeled with PKH26 Red (See, e.g., Bratosin, et al., (1997) Cytometry 30:269-274, which is incorporated herein by reference). Red blood cells (1-3×10⁷ cells) are suspended in 1 ml of “diluent C” and rapidly added to 1 ml or 2 _{μM PKH}26 dissolved in “diluent C”. The mixture is mixed by gentle pipetting and incubated at 25° C. for 2-5 min with constant stirring. The labeling may be stopped by adding an equal volume of human serum or compatible protein solution (e.g., 1% bovine serum albumin). After an additional minute, an equal volume of cell culture medium is added and the cells are isolated by centrifugation at 2000×g for 5 min, for example. Cells are washed three times by repeated suspension in cell culture medium and centrifugation. PHK26-labeled cells may be monitored with a maximum excitation wavelength of 551 nm and a maximum emission wavelength of 567 nm.

VivoTag 680 (VT680; VisEn Medical, Woburn, Mass., USA) may be used to track cells in vivo. VT680 is a near-infrared fluorochrome with a peak excitation wavelength of 670±5 nm and a peak emission wavelength of 688±5 nm. VT680 also contains an amine reactive NHS ester which enables it to cross-link with proteins and peptides. As such, the surface of cells may be labeled with VT680 (See, e.g., Swirski, et al., (2007) PloS ONE 10:e1075). For example, 4×10⁶ cells/ml are incubated with VT680 diluted in complete culture medium at a final concentration of 0.3 to 300 μg/ml for 30 min at 37° C. The cells are washed twice with complete culture medium after labeling. Cells may be non-specifically labeled based on proteins expressed on the surface of the modified red blood cell. Alternatively, a specific protein may be labeled with VT680. In some instances, an antibody directed against a specific protein associated with the modified red blood cell may be used to selectively label cells. In other instances, a protein or peptide may be directly labeled with VT680 ex vivo and subsequently either attached to the surface of the cell or incorporated into the interior of the cell using methods described here in for the uptake of nucleic acids.

In vivo monitoring, for example, may be performed using, for example, the dorsal skin fold. As such, laser scanning microscopy may be performed using, for example, an Olympus IV 100 in which VT680 is excited with a red laser diode of 637 mn and detected with a 660/LP filter. Alternatively, multiphoton microscopy may be performed using, for example, a BioRad Radiance 2100 MP centered around an Olympus BX51 equipped with a 20×/0.95 NA objective lens and a pulsed Ti:Sapphire laser tuned to 820 nm. The latter wavelength is chosen because VT680 has a peak in its two-photon cross-section at 820 nm.

Alternatively, a modified red blood cell may be labeled with other red and/or near-infrared dyes including, for example, cyanine dyes such as Cy5, Cy5.5, and Cy7 (Amersham Biosciences, Piscataway, N.J., USA) and/or a variety of Alexa Fluor dyes including Alexa Fluor 633, Alexa Fluor 635, Alexa Fluor 647, Alexa Fluor 660, Alexa Fluor 680, Alexa Fluor 700 and Alexa Fluor 750 (Molecular Probes-Invitrogen, Carlsbad, Calif., USA). Additional fluorophores include IRD41 and IRD700 (LI-COR, Lincoln, Nebr., USA), NIR-1 and 105-OSu (Dejindo, Kumamotot, Japan), LaJolla Blue (Diatron, Miami, Fla., USA), FAR-Blue, FAR-Green One, and FAR-Green Two (Innosense, Giacosa, Italy), ADS 790-NS and ADS 821-NS (American Dye Source, Montreal, Calif.). Quantum dots (Qdots) of various emission/excitation properties may also be used for labeling cells (See, e.g., Jaiswal et al., Nature Biotech. 21:47-51 (2003), which is incorporated herein by reference). Many of these fluorophores are available from commercial sources either attached to primary or secondary antibodies or as amine-reactive succinimidyl or monosuccinimidyl esters, for example, ready for conjugation to a protein or proteins either on the surface or inside the red blood cells.

Magnetic nanoparticles may be used to track cells in vivo using high resolution MRI (Montet-Abou et al., Molecular Imaging 4:165-171 (2005), which is incorporated herein by reference). Magnetic particles may be internalized by several mechanisms. Magnetic particles may be taken up by a cell through fluid-phase pinocytosis or phagocytosis. Alternatively, the magnetic particles may be modified to contain a surface agent such as, for example, the membrane translocating HIV tat peptide which promotes internalization. In some instances, a magnetic nanoparticle such as, for example, Feridex IV®, an FDA approved magnetic resonance contrast reagent, may be internalized into hematopoietic cells in conjunction with a transfection agent such as, for example, protamine sulfate (PRO), polylysine (PLL), and lipofectamine (LFA).

### V. Formulations of Pharmaceutical Compositions

The modified red blood cells can be incorporated into pharmaceutical compositions suitable for administration. The pharmaceutical compositions generally comprise substantially purified modified red blood cells and a pharmaceutically-acceptable carrier in a form suitable for administration to a subject. Pharmaceutically-acceptable carriers are determined in part by the particular composition being administered, as well as by the particular method used to administer the composition. Accordingly, there is a wide variety of suitable formulations of pharmaceutical compositions for administering the antibody compositions (See, e.g., Remington's Pharmaceutical Sciences, Mack Publishing Co., Easton, Pa. 18^{th} ed. (1990), which is incorporated herein by reference). The pharmaceutical compositions are generally formulated as sterile, substantially isotonic and in full compliance with all Good Manufacturing Practice (GMP) regulations of the U.S. Food and Drug Administration.

The terms “pharmaceutically-acceptable,” “physiologically-tolerable,” and grammatical variations thereof, as they refer to compositions, carriers, diluents and reagents, are used interchangeably and include materials are capable of administration to or upon a subject without the production of undesirable physiological effects to a degree that would prohibit administration of the composition. For example, “pharmaceutically-acceptable excipient” includes an excipient that is useful in preparing a pharmaceuticalcomposition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

Examples of such carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the modified red blood cells, use thereof in the compositions is contemplated. Supplementary active compounds can also be incorporated into the compositions. A pharmaceutical composition is formulated to be compatible with its intended route of administration. The modified red blood cells can be administered by parenteral, topical, intravenous, oral, subcutaneous, intraarterial, intradermal, transdermal, rectal, intracranial, intraperitoneal, intranasal; intramuscular route or as inhalants. The modified red blood cells can optionally be administered in combination with other agents that are at least partly effective in treating various diseases including various actin- or microfilament-related diseases.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. In many cases, it will be preferable to include isotonic compounds, e.g., sugars, polyalcohols such as manitol, sorbitol, sodium chloride in the composition. Prolonged absorption of the injectable compositions can be brought about by including in the composition a compound which delays absorption, e.g., aluminum monostearate and gelatin.

Sterile injectable solutions can be prepared by incorporating the modified red blood cells in the required amount in an appropriate solvent with one or a combination of ingredients enumerated above, as required. Generally, dispersions are prepared by incorporating the modified red blood cells into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. The modified red blood cells can be administered in the form of a depot injection or implant preparation which can be formulated in such a manner as to permit a sustained or pulsatile release of the active ingredient.

Oral compositions generally include an inert diluent or an edible carrier. They can be enclosed in gelatin capsules or compressed into tablets. For the purpose of oral therapeutic administration, the modified red blood cells can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

For administration by inhalation, the modified red blood cells are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of nasal sprays or suppositories. For transdermal administration, the modified red blood cells are formulated into ointments, salves, gels, or creams as generally known in the art.

The modified red blood cell an also be prepared as pharmaceutical compositions in the form of suppositories (e.g., with conventional suppository bases such as cocoa butter and other glycerides) or retention enemas for rectal delivery.

In an embodiment, the modified red blood cells are prepared with carriers that will protect the modified red blood cells against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically-acceptable carriers. These can be prepared according to methods known to those skilled in the art, e.g., as described in U.S. Pat. No. 4,522,811, which is incorporated herein by reference.

In an embodiment, the artificial antigen presenting cell includes a polymeric vehicle. In an embodiment, the polymeric vehicle includes at least one of polyester, polylactic acid, polylactic-co-gylcolic acid, cellulose, nitrocellulose, urea, urethane, or other polymer. In an embodiment, the polymeric vehicle is made by exposure to alcohol. The dehydrated polymeric vehicles are then utilized as a substrate upon which the artificial antigen presenting cell complex is deposited in layers. In an embodiment, the artificial antigen presenting cell complex includes one or more proteins, which are, in certain cases, crosslinked in order to anchor them to the polymeric vehicle. In certain cases, the inner structure is then dissolved and the outer, flexible skeleton remains. See, for example, Doshi, et al., Proc. Natl Acad. Sci., 106 (51): 21495-21499 (2009), which is incorporated herein by reference.

### Methods of Use

### I. General Methods of Targeting Cells or Tissues

This section will generally describe an embodiment of the methods of using the modified red blood cell compositions. Further details of particular applications are described in the sections that follow.

In one aspect, the disclosure provides methods of using a modified red blood cell to bind a target molecule, thereby localizing the modified red blood cell to a particular location (e.g., tissue or cell) within a subject. In an embodiment, the fusion protein is configured to facilitiate fusion of the red blood cell with the target cell. In other embodiments, upon binding of the target recognition moiety to the target molecule, the complex becomes activated. As such, a singlet oxygen molecule can be delivered to the particular location (e.g., tissue or cell) by exposing the area to light of a suitable wavelength. The disclosure also provides for methods of using a modified red blood cell to bring a target cell or tissue in contact with a molecular agent, which is carried by the modified red blood cell. For example, the artificial antigen presenting cell complex is configured to target a T cell. In an embodiment, the modified red blood cells may be useful for the treatment of infection (e.g., bacterial, fungal, viral or parasitic) or for the treatment of cancer or other hyperproliferative disorders (e.g., restenosis or benign prostatic hyperplasia), by damaging or destroying the target cells.

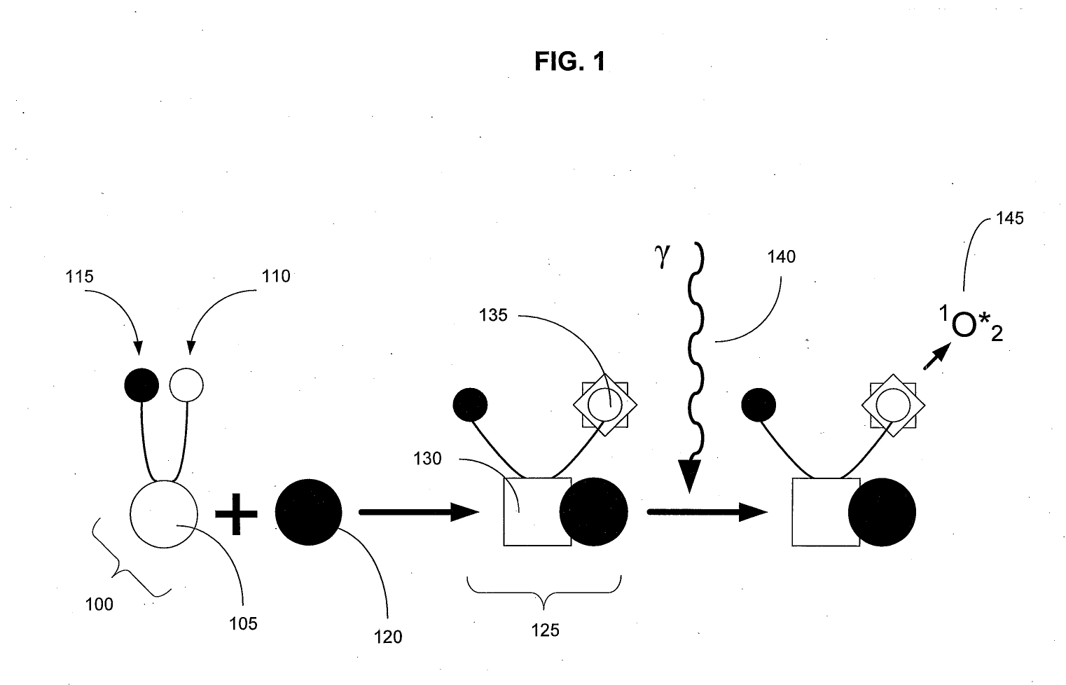

As shown in FIG. 1, a target-binding agent 100 is composed of a target recognition moiety 105, a photoactivatable molecule 110, and a quencher molecule 115. In the absence of a target, the photoactivatable molecule 110 is in close proximity to the quencher molecule 115 and is not responsive by light. In the presence of a target 120, an activated unit 125 is generated. As such, the target recognition moiety 105 undergoes a conformational change 130. The quencher molecule 115 moves away from the photoactivatable molecule 110, resulting in an activated photoactivatable molecule 135. In response to light energy 140, the activated photoactivatable molecule 135 emits a reactive singlet oxygen 145.

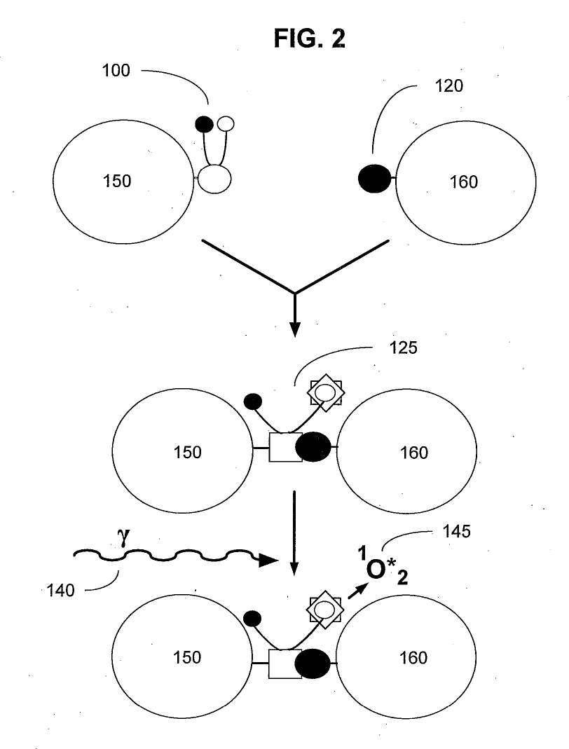

As shown in FIG. 2, one or more red blood cells 150 may be modified with a target-binding agent 100 to form a modified red blood cell. The modified red blood cells can be released into the blood circulation of the subject. In circulation, the one or more modified red blood cells 150 may come into contact with a target cell 160 which expresses on its surface, for example, a target 120 recognized by the target recognition moiety of the target-binding agent. The target cell 160 may be a pathogen such as for example a bacterium, a fungus, or a parasite. Alternatively, the target cell 160 may be a cancerous cell such as for example a leukemia cell, a circulating tumor cell (CTC), or other cancerous cell. Upon binding, the target-binding agent is converted into an activated unit. In response to light energy 140, the activated unit 125 releases a reactive singlet oxygen 145.

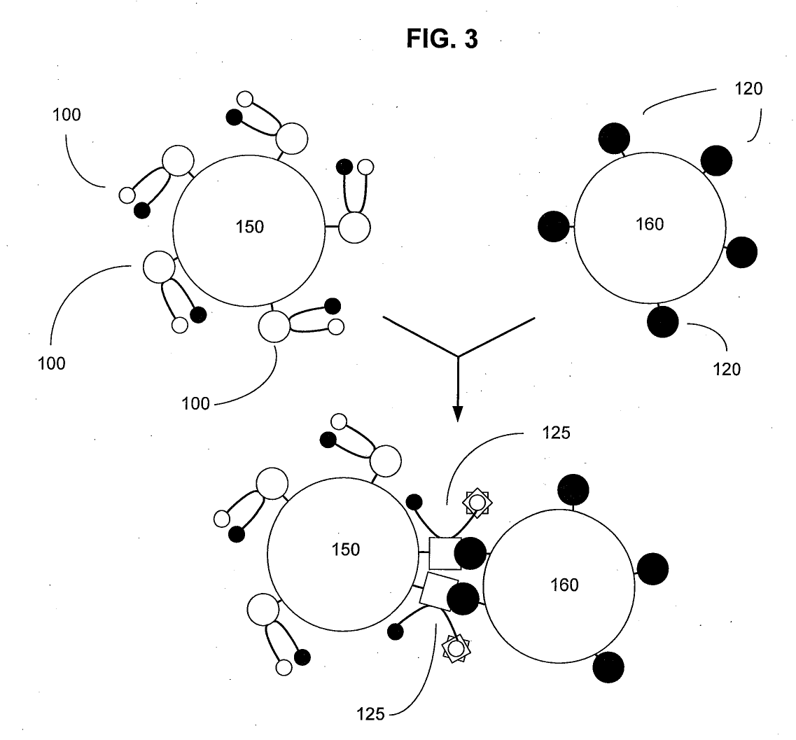

As shown in FIG. 3, one or more red blood cells 150 may be modified with one or more target-binding agents 100 to form a modified red blood cell. Similarly, one or more target cells 160 may have one or more targets 120 recognized by the target-binding agent 100. As such, one or more targets 120 may bind to one or more target-binding agents 100 to generate one or more activated units 125.

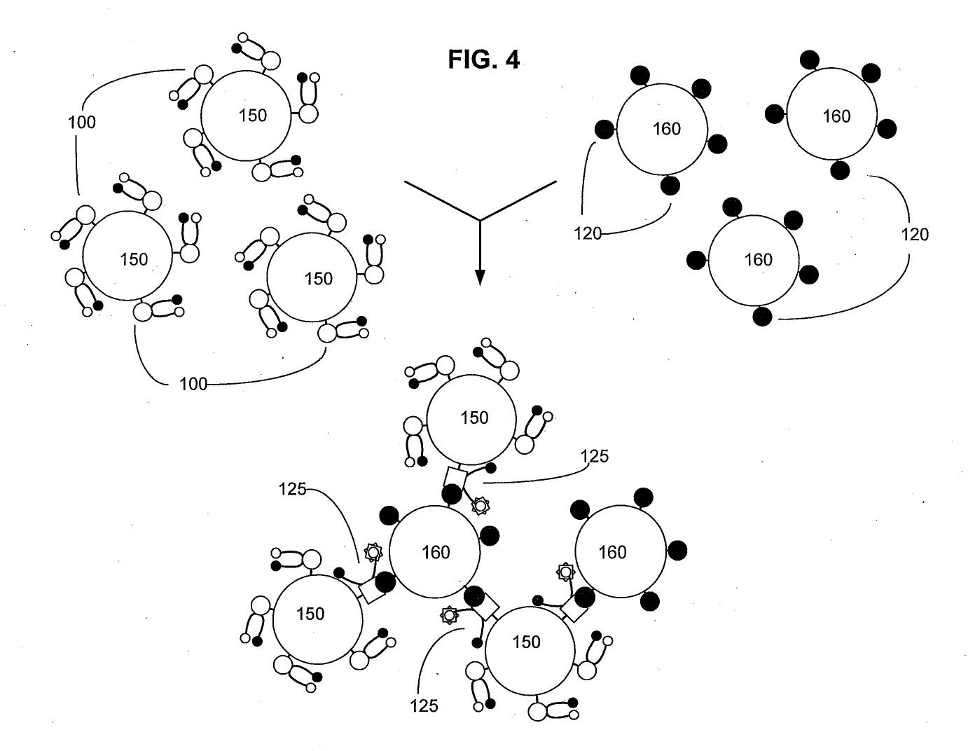

As shown in FIG. 4, one or more modified red blood cells 150 modified with one or more target-binding agents 100 may interact with one or more target cells 160 with one or more targets 120 recognized by the target-binding agent 100. As such, one or more modified red blood cells 150 may interact with one or more target cells. Singlet oxygen may produce cellular damage. Since the singlet oxygen has a very short lifetime (microseconds), the photodamage can be expected to be within a short radius of the target molecule.

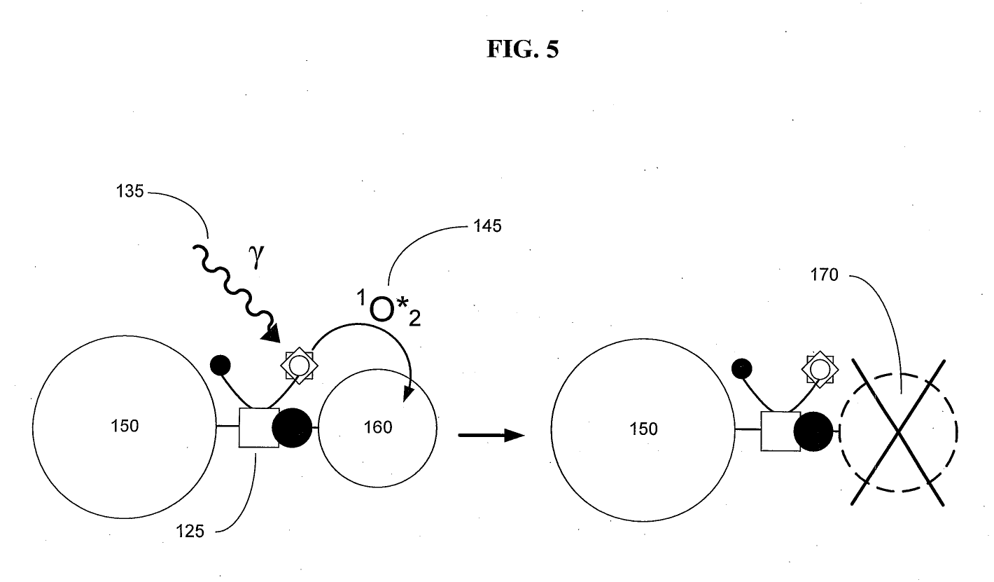

As shown in FIG. 5, upon binding of a modified red blood cell 150 to a target cell 160, the target-binding agent is converted into the activated unit 125. The activated unit 125 may now be excited by light energy 135 resulting in' release of a reactive singlet oxygen 145. The reactive singlet oxygen 145 may, in turn, interact with the target cell 160. The reactive singlet oxygen 145 may cause apoptosis and/or necrosis of the target cell 160 leading to a dead or inactivated target cell 170.

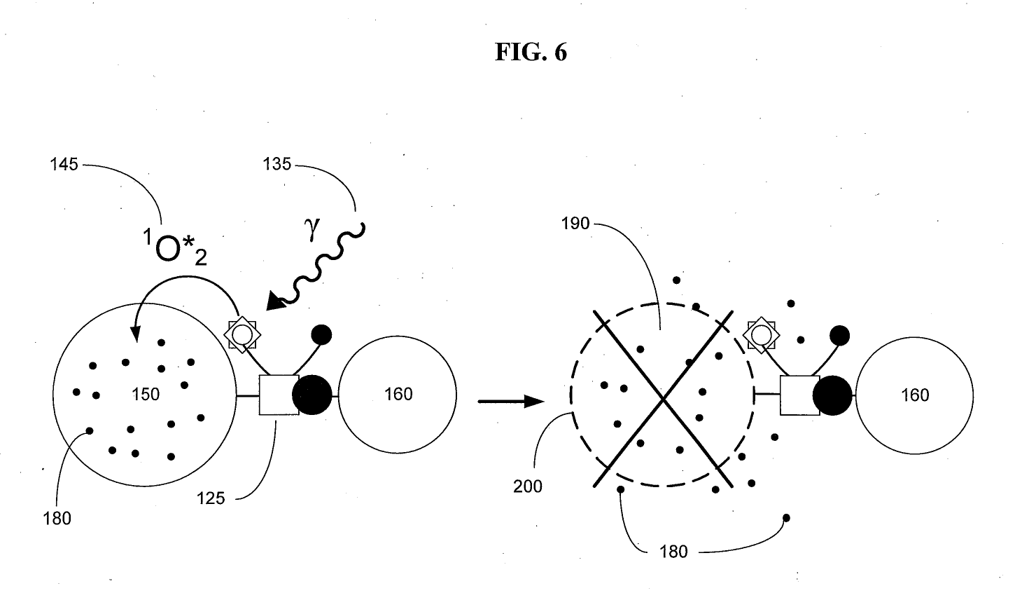

As shown in FIG. 6, in some instances, the modified red blood cells 150 may be loaded with a therapeutic agent 180. A therapeutic agent 180 may be a small molecule drug, a biological drug such as, for example, an antibody, a ligand, a receptor and/or an enzyme, or an oligonucleotide such as, for example, an RNA or DNA aptamer, an interfering RNA, and/or an antisense RNA. Upon binding of a modified red blood cell 150 to a target cell 160, the target-binding agent is converted into the activated unit 125. The activated unit 125 may now be excited by light energy 135 resulting in release of a reactive singlet oxygen 145. The reactive singlet oxygen 145 may, in turn, interact with the modified red blood cell 150. The reactive singlet oxygen 145 may cause apoptosis and/or necrosis of the modified red blood cell 150 resulting in a dead or inactivated red blood cell 190. The inactivated modified red blood cell 190 releases the therapeutic agent through the compromised cell membrane 200 in proximity of the target cell 160.

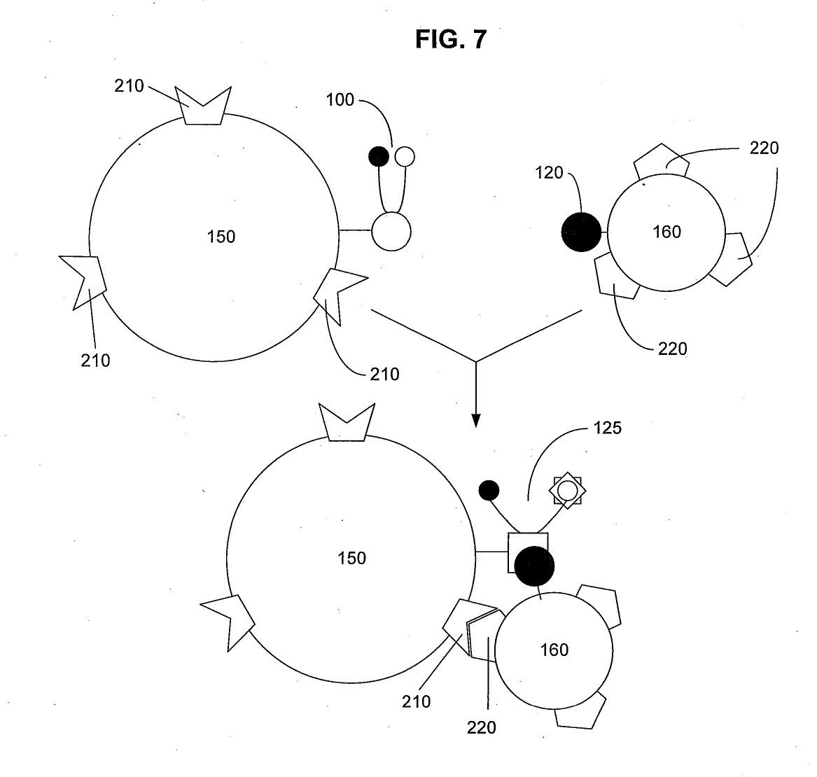

As shown in FIG. 7, in some instances, the modified red blood cells 150 comprising one or more target-binding agents 100 may also be modified with another target recognition moiety 210. The target recognition moiety 210 may recognize a receptor 220 on the target cell 160. The target recognition moiety 210 and the receptor 220 may be an antibody and an antigen, respectively. Alternatively, the target recognition moiety 210 and the receptor 220 may be a ligand/receptor pair. As such, modified red blood cells 150 and one or more target cells 160 may interact through the target-binding agent 100 and the target 120 to form the activated unit 125, through the target recognition moiety 210 and the receptor 220, or through a combination of both to facilitate a more selective interaction, for example.

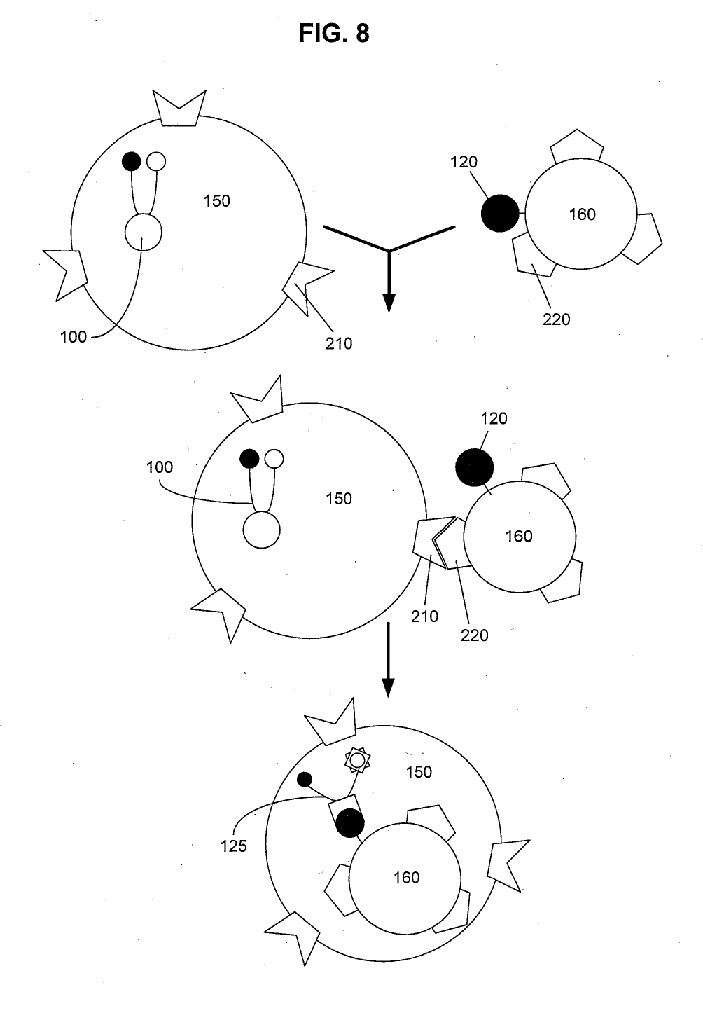

As shown in FIG. 8, in some instances, one or more target-binding agents 100 may be loaded into the cytoplasm, for example, of one or more red blood cells 150 to form a modified red blood cell. The modified red blood cell may be further modified with another target recognition moiety 210. The target recognition moiety 210 may recognize a receptor 220 on the target cell 160. In some instances, the interaction of the target recognition moiety 210 and the receptor 220 may lead to fusion and/or invasion of the red blood cell 150 by the target cell 160. As such, the modified red blood cell 150 may take up the target cell 160. Inside the modified red blood cell 150, the target cell 120 may interact with the target-binding agent 100 to generate the activated unit 125. The internalized target cell 160 may then be damaged or destroyed when the activated unit is exposed to light of an appropriate wavelength and power and a singlet oxygen radical molecule is produced.

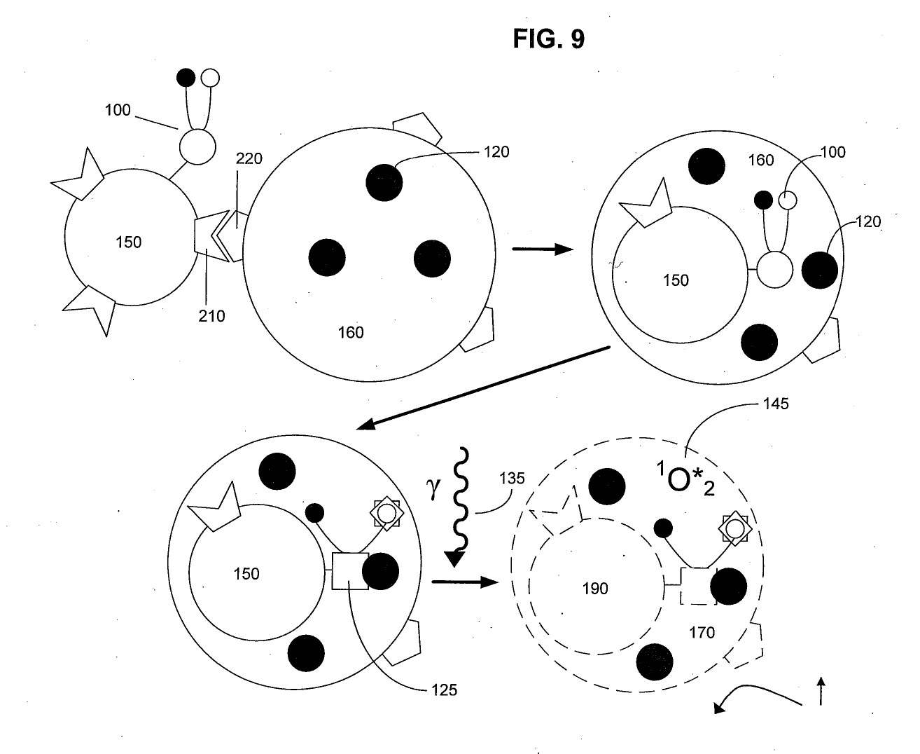

As shown in FIG. 9, in some instances, the modified red blood cells 150 modified with one or more target-binding agent 100 may also be modified with another target recognition moiety 210. The target recognition moiety 210 may recognize a receptor 220 on the target cell 160. In some instances, the interaction of the target recognition moiety 210 and the receptor 220 may lead to fusion of the red blood cell 150 and the target cell 160. Inside the target cell 160, the target-binding agent 100 may interact with the target 120 to generate the activated unit 125. Upon radiation with light energy 135, the activated unit 125 emits reactive singlet oxygen leading to a damaged or destroyed target cell 170 and/or damaged or destroyed red blood cell 190.

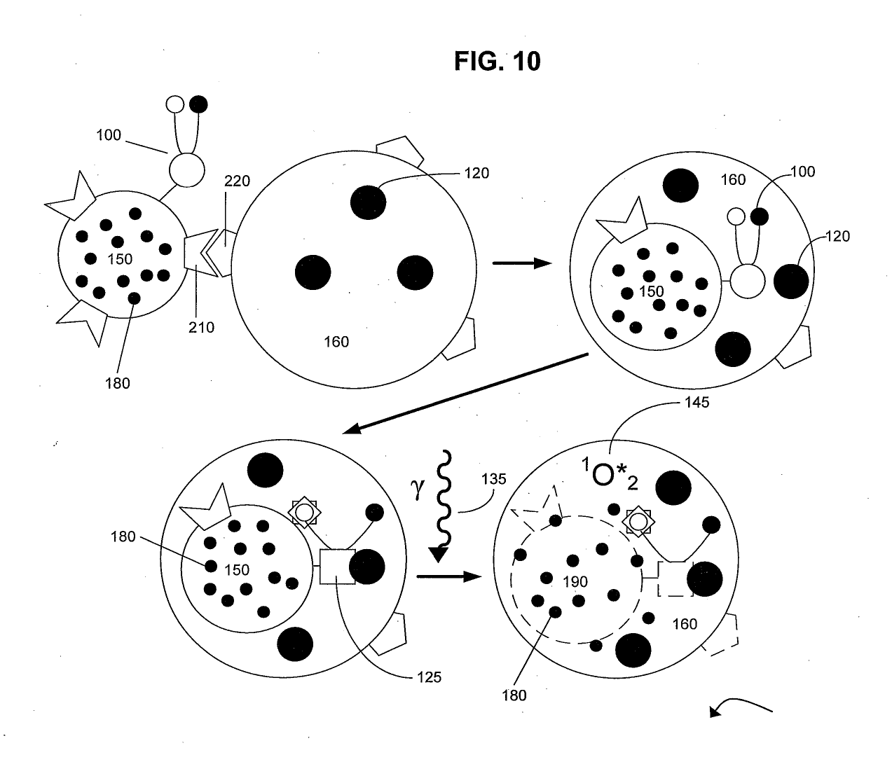

As shown in FIG. 10, in some instances, the modified red blood cell 150 modified with one or more target-binding agent 100 and one or more target recognition moiety 210 may be loaded with a therapeutic agent 180. The target recognition moiety 210 may recognize a receptor 220 on the target cell 160. In some instances, the interaction of the target recognition moiety 210 and the receptor 220 may lead to fusion of the red blood cell 150 and the target cell 160. Inside the target cell 160, the target-binding agent 100 may interact with the target 120 to generate the activated unit 125. Upon radiation with light energy 135, the activated unit 125 emits reactive singlet oxygen leading to damaged or destroyed red blood cell 190 and release of the therapeutic agent 180 into the cytoplasm of the target cell 160.

As shown in FIG. 11, in an embodiment 11.A, the at least one epitope 118 joined to the at least one MHC receptor component 115, is also joined to at least one immunomodulatory component 110, by an intracellular joining mechanism (e.g.,linker, etc.). Also shown in 11A is a bi-functional or bi-directional artificial antigen presenting cell complex. As described herein, the artificial antigen presenting cell complex includes, in an embodiment, at least two different MHC:epitope combinatorial structures, providing a bi-functional complex. In an embodiment, the MHC:epitope combinatorial structures are directed toward different angles (e.g., one facing the inner surface, and one facing the outer surface) providing a bi-directional complex. In an embodiment, the artificial antigen presenting cell complex is both bi-directional and bi-functional. In an embodiment 11B, the at least one epitope 118 joined to the at least one MHC receptor component 115, is also joined to at least one immunomodulatory component 110 by way of an extracellular joining mechanism (e.g., linker, etc. In an embodiment 11C, the at least one epitope 118 joined to the at least one MHC receptor component 115, is also joined to at least one immunomodulatory component 110 by way of a joining mechanism embedded within the lipid surface (e.g., cell membrane, polymeric layer, etc.). In an embodiment 11C, the artificial antigen presenting cell complex (e.g., epitope joined to an MHC receptor component) is displayed on the interior of the cell or other vehicle, until it is displayed on the outer surface, or is released from the interior.

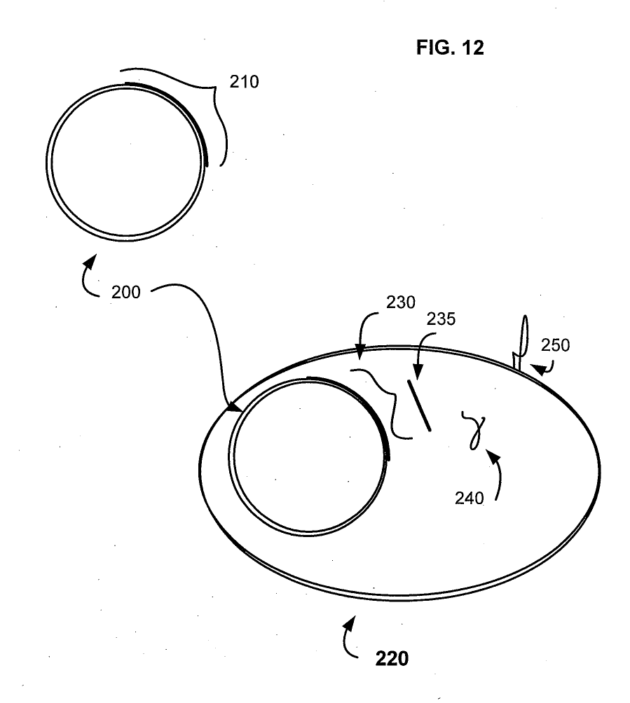

As shown in FIG. 12, in an embodiment, a vector 200 including at least one regulatory nucleic acid construct 210 is placed into at least one cell 220 by methods known in the art (e.g., electroporation, transformation, etc.). Once incorporated into the cell 220, the regulatory nucleic acid construct 210 either inhibits proper gene expression of at least one endogenous histocompatibility antigen related gene 235 (e.g., shRNA, iRNA, microRNA, etc.), or is transcribed, resulting in production of at least one transcript 230, which is converted intracellularly into at least one protein 240 (e.g, a dominant negative form of MHC or other histocompatibility antigen related gene). In an embodiment, the protein can remain intracellularly 240, or be expressed on the surface 250 of the cell 220.

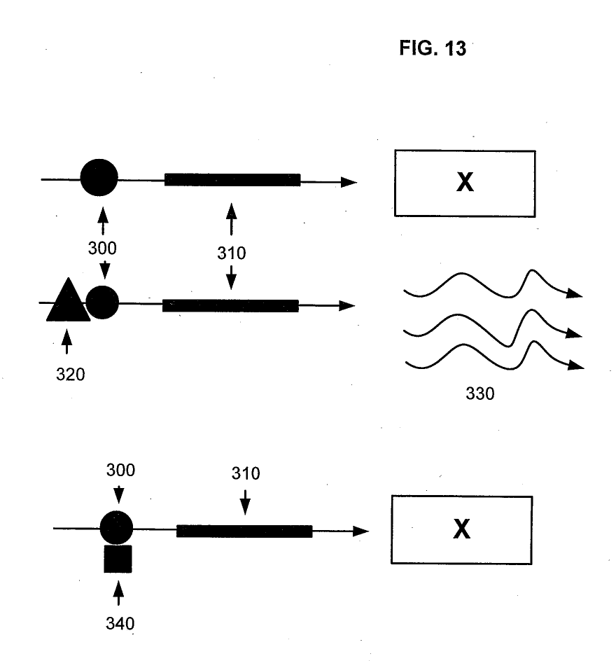

As illustrated in FIG. 13, an example of a nucleic construct, including an inducible promoter 300 is capable of regulating expression of at least one gene 310. In the absence of an inducer 320, the gene 310 is not transcribed (as indicated by the “X”). However, in the presence of the inducer 320, the promoter 300 directs transcription of the gene 310, resulting in production of at least one transcript 330. Likewise, in the presence of a repressor 340, the promoter 300 does not support gene transcription of the gene 310 (as indicated by the “X”).

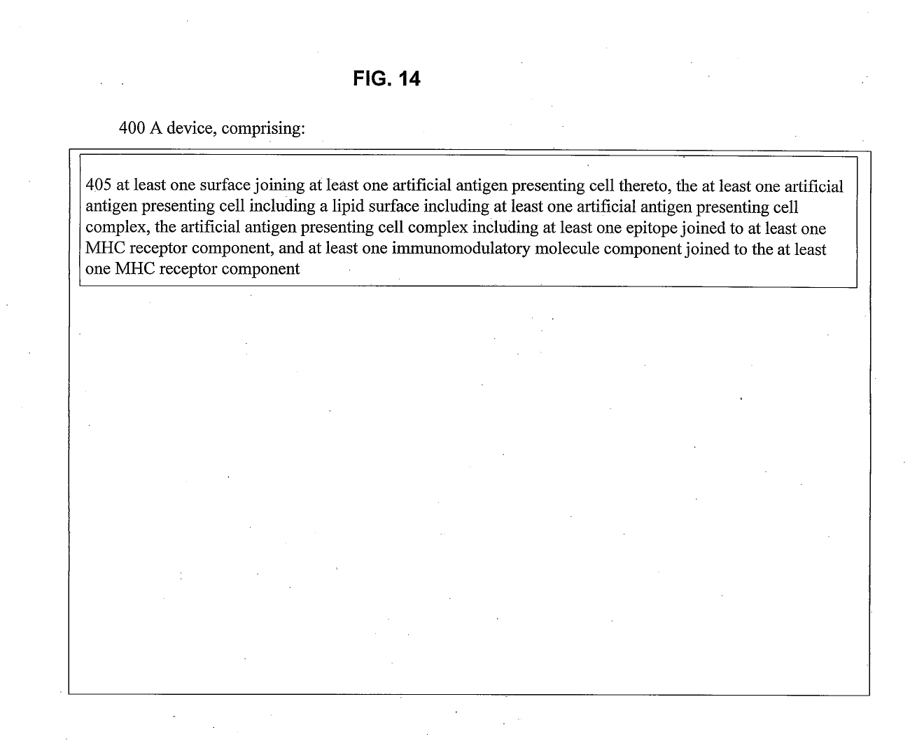

As shown in FIG. 14, in an embodiment, a device 400 includes 405 at least one surface joining at least one artificial antigen presenting cell thereto, the at least one artificial antigen presenting cell including a lipid surface including at least one artificial antigen presenting cell complex, the artificial antigen presenting cell complex including at least one epitope joined to at least one MHC receptor component, and at least one immunomodulatory molecule component joined to the at least one MHC receptor component.

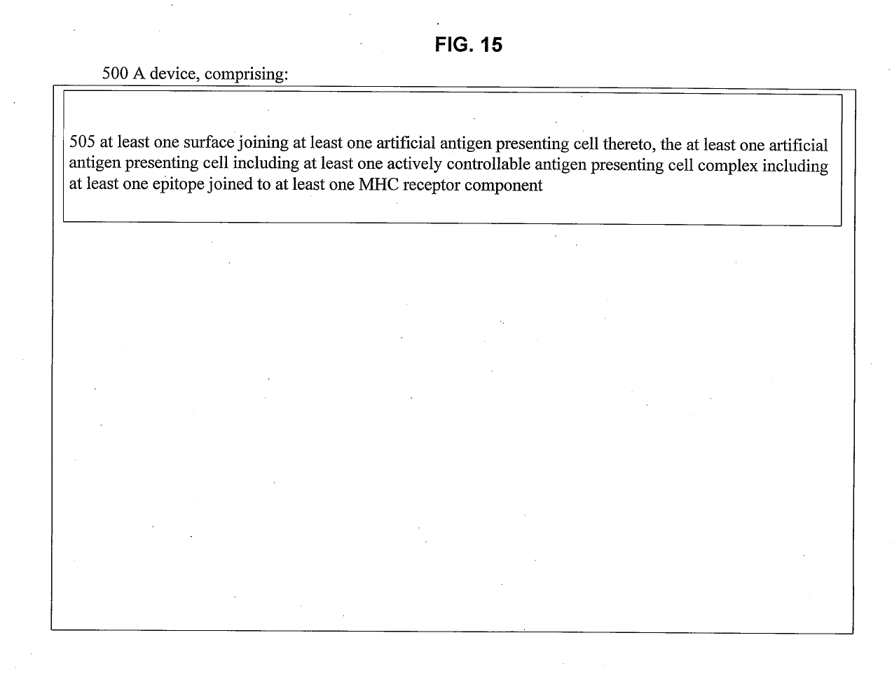

As shown in FIG. 15, in an embodiment, a device 500 includes 505 at least one surface joining at least one artificial antigen presenting cell thereto, the at least one artificial antigen presenting cell including at least one actively controllable antigen presenting cell complex including at least one epitope joined to at least one MHC receptor component.

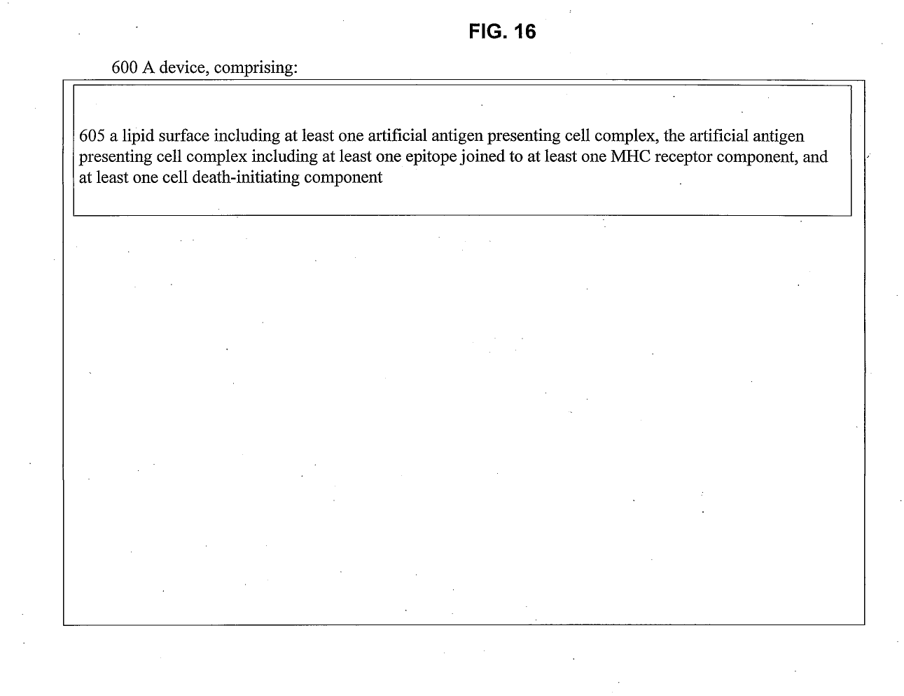

As shown in FIG. 16, in an embodiment, a device 600, includes 605, at least one surface joining at least one artificial antigen presenting cell thereto, the at least one artificial antigen presenting cell including a modified cell including at least one artificial antigen presenting cell complex, the at least one artificial antigen presenting cell complex including at least one epitope joined to at least one MHC receptor component; and at least one nano-tube operably linked to a NKG2D receptor on the modified cell

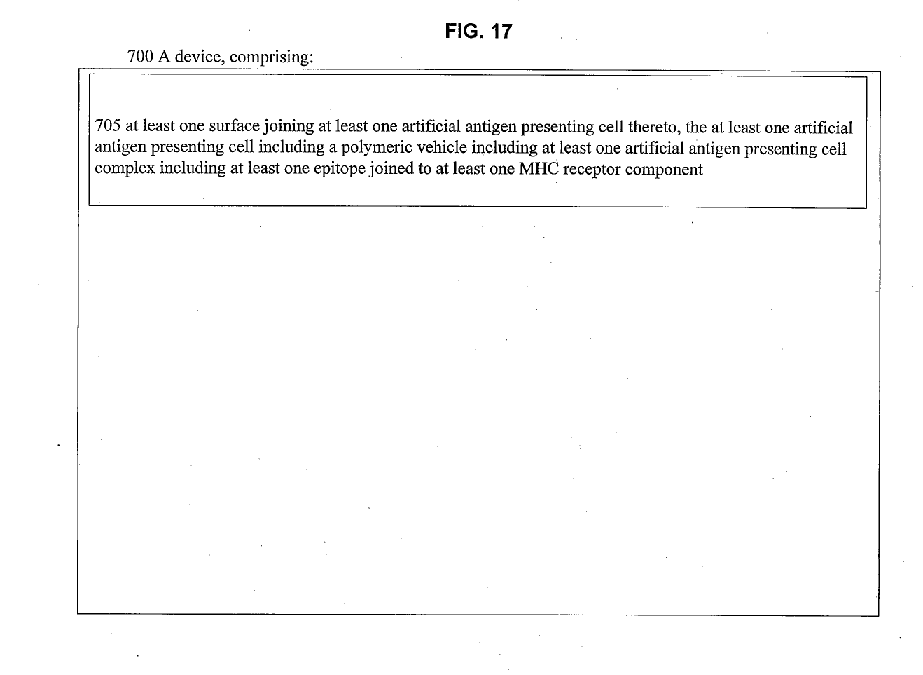

As shown in FIG. 17, in an embodiment, a device 700, includes 705, at least one surface joining at least one artificial antigen presenting cell thereto, the at least one artificial antigen presenting cell including a polymeric vehicle including at least one artificial antigen presenting cell complex including at least one epitope joined to at least one MHC receptor component

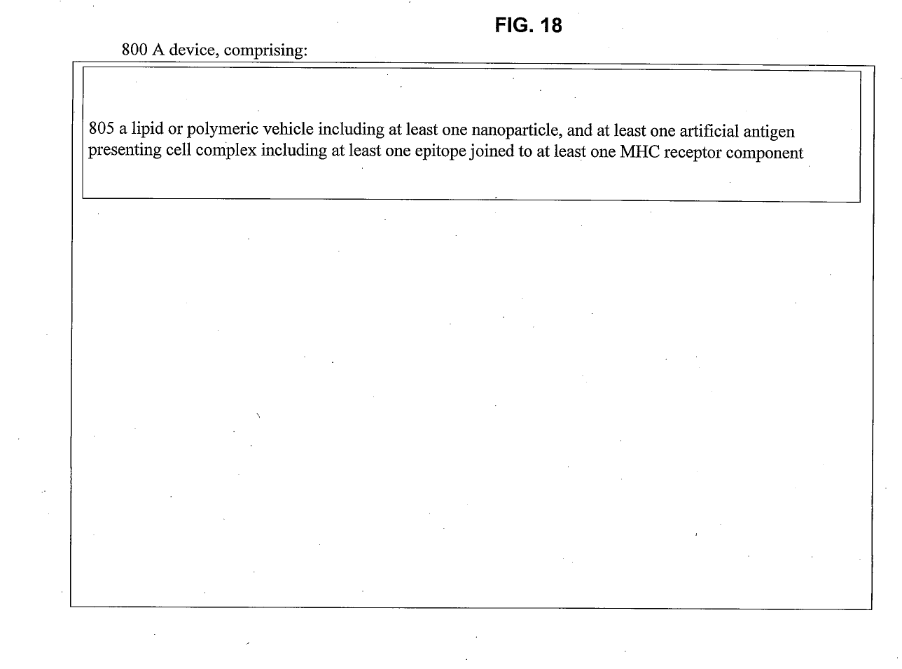

As shown in FIG. 18, in an embodiment, a device 800, includes 805, a liposome including at least one nanoparticle, and at least one artificial antigen presenting cell complex including at least one epitope joined to at least one MHC receptor component.

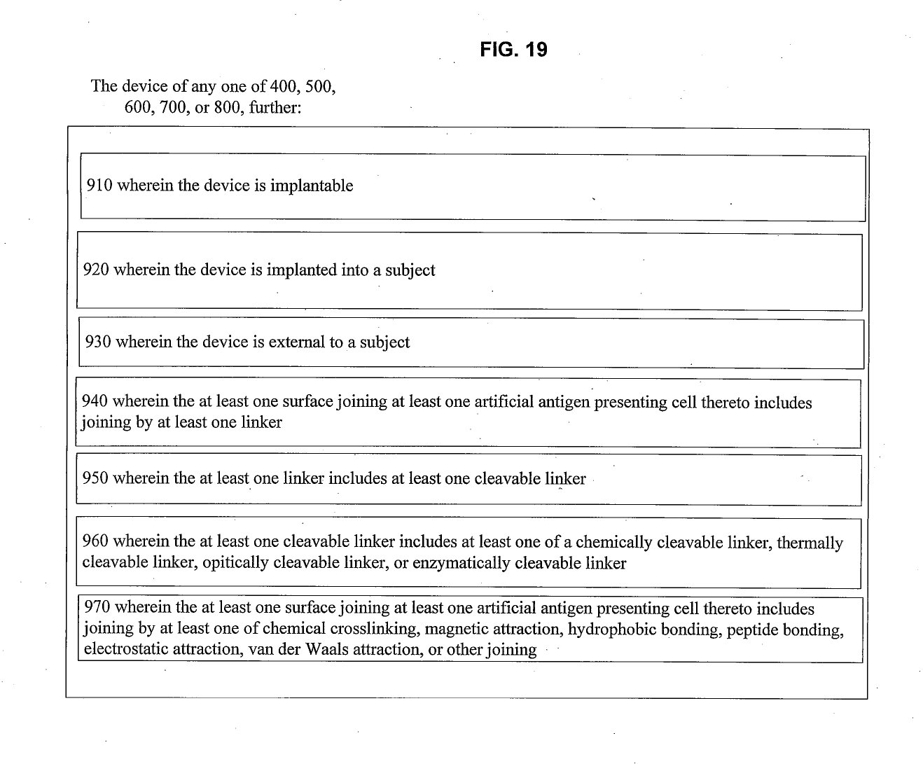

As shown in FIG. 19, in particular embodiments, the device of any of 400, 500, 600, 700, or 800 further include 910 wherein the device is implantable. In an embodiment 920, the device is implanted into a subject. In an embodiment 930, the device is external to a subject. In an embodiment 940, the at least one surface joining at least one artificial antigen presenting cell thereto includes joining by at least one linker or linking component, as described herein. In an embodiment 950, the at least one linker includes at least one cleavable. linker. In an embodiment 960, the at least one cleavable linker includes at least one of a chemically cleavable linker, thermally cleavable linker, optically cleavable, or enzymatically cleavable linker. In an embodiment 970, the at least one surface joining at least one artificial antigen presenting cell thereto includes joining by at least one of a chemical crosslinking, magnetic attraction, hydrophobic bonding, peptide bonding, elctrostatci attraction, van der Waals attraction, or other joining.

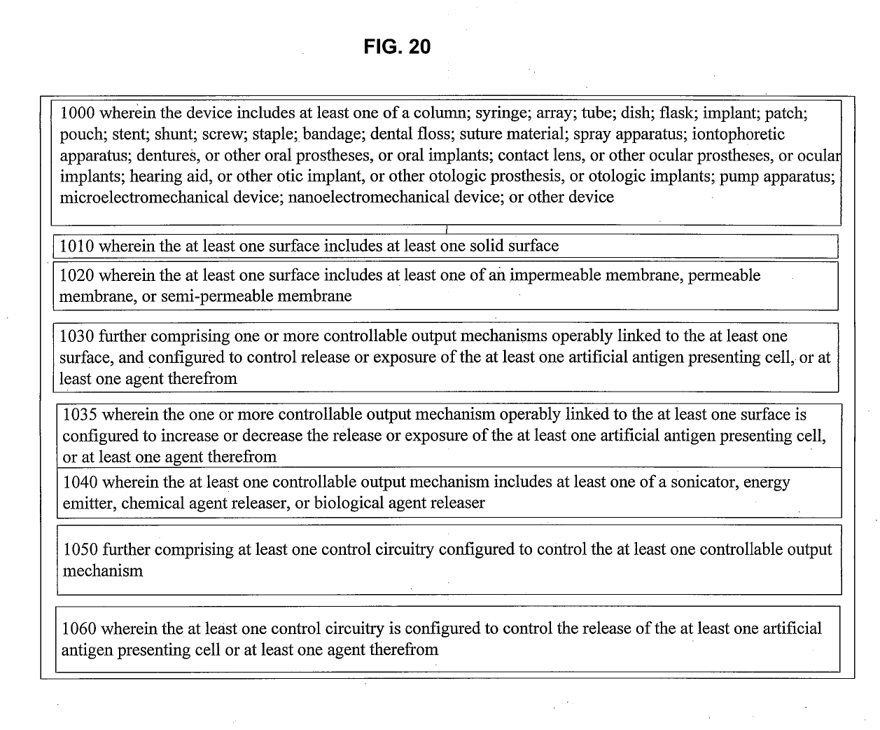

As shown in FIG. 20, in particular embodiments, the device of any of 400, 500, 600, 700, or 800 further include 1000 the device includes at least one of a column; syringe; array; tube; dish; flask; implant; patch; pouch; stent; shunt; screw; staple; bandage; dental floss; suture material; spray apparatus; iontophoretic apparatus; dentures, or other oral prostheses, or oral implants; contact lens, or other ocular prostheses, or ocular implants; hearing aid, or other otic implant, or other otologic prosthesis, or otologic implants; pump apparatus; microelectromechanical device; nanoelectromechanical device; or other device. In an embodiment 1010, the at least one surface includes at least one solid surface. In an embodiment 1020, the at least one surface includes at least one of an impermeable membrane, permeable membrane, or semi-permeable membrane. In an embodiment 1030, the device further comprises one or more controllable output mechanisms operably linked to the at least one surface, and configured to control release or exposure of the at least one artificial antigen presenting cell or at least one agent therefrom. In an embodiment 1035 the one or more controllable output mechanism operably linked to the at least one surface is configured to increase or decrease the release or exposure of the at least one artificial antigen presenting cell or at least one agent therefrom. In an embodiment 1040, the at least one controllable output mechanism includes at least one of a sonciator, energy emitter, chemical agent releaser, or biological agent releaser. In an embodiment 1050, the device further includes at least one control circuitry configured to control the at least one controllable output mechanism. In an embodiment 1060, the at least one control circuitry is configured to control the release of the at least one artificial antigen presenting cell or at least one agent therefrom.

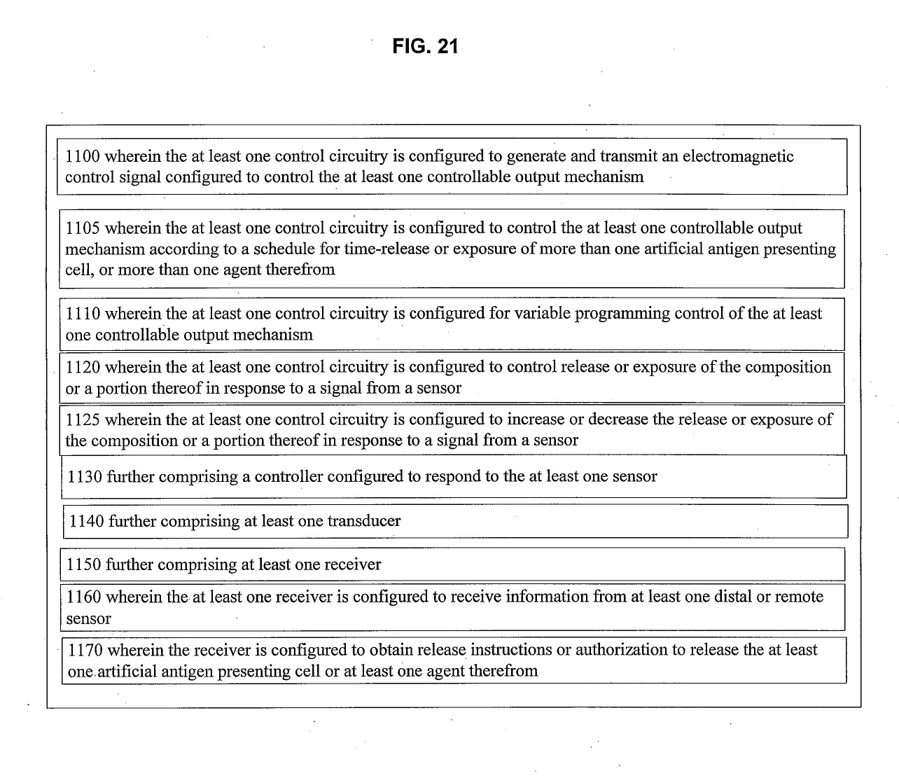

As shown in FIG. 21, in particular embodiments, the device of any of 400, 500, 600, 700, or 800 further include 1100 wherein the at least one control circuitry is configured to generate and transmit an electromagnetic control signal configured to control the at least one controllable output mechanism. In an embodiment, 1105 the at least one control circuitry is configured to control the at least one controllable output mechanism according to a schedule for time-release or exposure of more than one artificial antigen presenting cell, or more than one agent therefrom. In an embodiment, 1110 the at least one control circuitry is configured for variable programming control of the at least one controllable output mechanism. In an embodiment, 1120 the at least one control circuitry is configured to control release or exposure of the composition or a portion thereof in response to a signal from a sensor. In an embodiment, 1125 the at least one control circuitry is configured to increase or decrease the release or exposure of the composition or a portion thereof in response to a signal from a sensor. In an embodiment, 1130 the device further includes a controller configured to respond to the at least one sensor. In an embodiment, 1140 the device further includes at least one transducer. In an embodiment, 1150 the device further includes at least one receiver. In an embodiment 1160 wherein the at least one receiver is configured to receive information from at least one distal or remote sensor. In an embodiment, 1170 wherein the receiver is configured to obtain release instructions or authorization to release the at least one artificial antigen presenting cell or at least one agent therefrom.

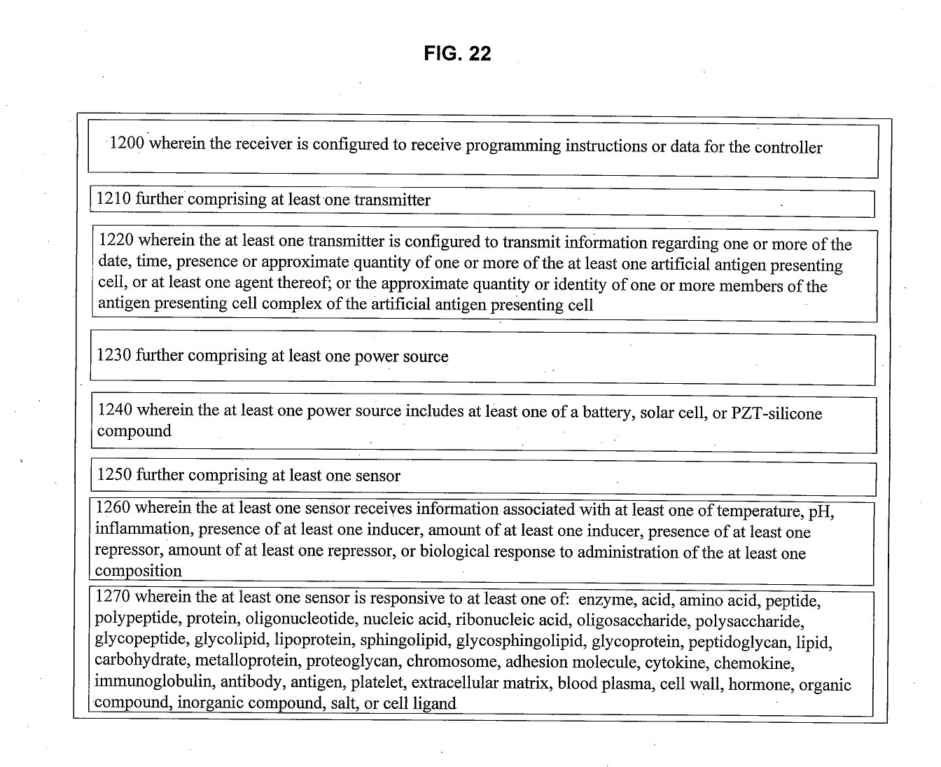

As shown in FIG. 22, in particular embodiments, the device of any of 400, 500, 600, 700, or 800 further include 1200 wherein the receiver is configured to receive programming instructions or data for the controller. In an embodiment 1210, the device further comprises at least one transmitter. In an embodiment 1220, the at least one transmitter is configured to transmit information regarding once or more of the date, time, presence or approximate quantity of oen or more of the at least one artifician antigen presenting cell, or at least one agent thereof; or the approximate quantity or identity of one or more members of the antigen presenting cell complex of the artificial antigen presenting cell. In an embodiment 1230, the device further comprises at least one power source. In an embodiment 1240, the at least one power source includes at least one of a battery, solar cell, or PXT-silicone compound. In an embodiment 1250, the device further includes at least one sensor. In an embodiment 1260, the at least one sensor receives information associated with at least one of temperature, pH, inflammation, presence of at least one inducer, amount of at least one inducer, presence of at least one repressor, amount of at least one repressor, or biological response to administration of the at least one composition. In an embodiment 1270, the at least one sensor is responsive to at least one of enzyme, acid, amino acid, peptide, polypeptide, protein, oligonucleotide, nucleic acid, ribonucleic acid, oligosaccharide, polysaccharide, glycopeptide, glycolipid, lipoprotein, sphingolipid, glycosphingolipid, glycoprotein, peptidoglycan, lipid, carbohydrate, metalloprotein, proteoglycan, chromosome, adhesion molecule, cytokine, chemokine, immunoglobulin, antibody, antigen, platelet, extracellular matrix, blood plasma, cell wall, hormone, organic compound, inorganic compound, salt, or cell ligand.

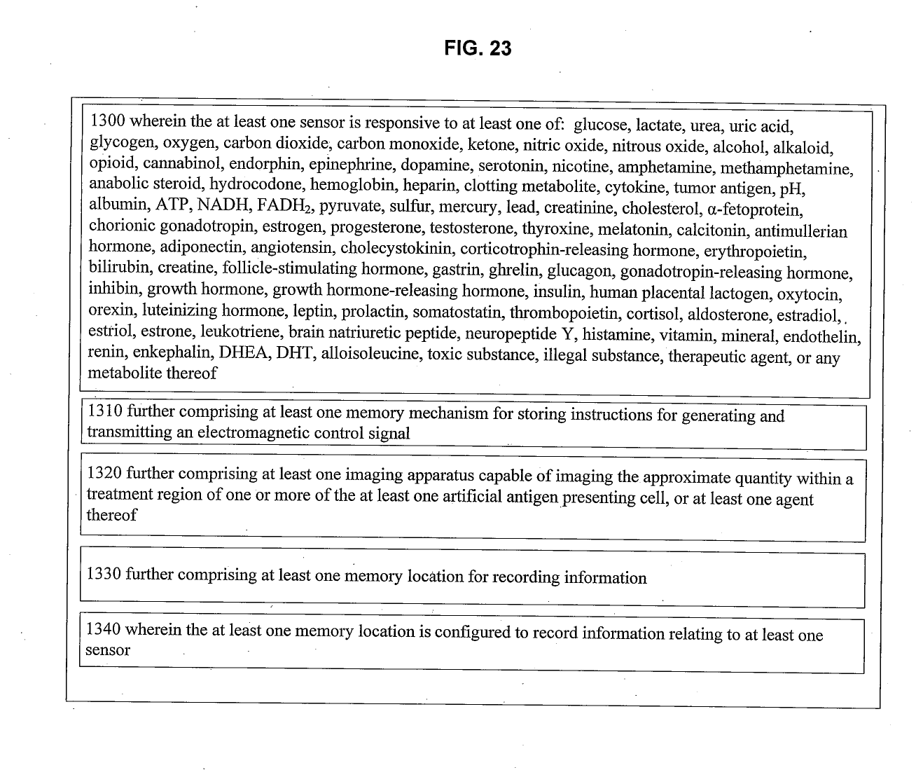

As shown in FIG. 23, in particular embodiments, the device of any of 400, 500, 600, 700, or 800 further include 1300 wherein the at least one sensor is responsive to at least one of: glucose, lactate, urea, uric acid, glycogen, oxygen, carbon dioxide, carbon monoxide, ketone, nitric oxide, nitrous oxide, alcohol, alkaloid, opioid, cannabinol, endorphin, epinephrine, dopamine, serotonin, nicotine, amphetamine, methamphetamine, anabolic steroid, hydrocodone, hemoglobin, heparin, clotting metabolite, cytokine, tumor antigen, pH, albumin, ATP, NADH, FADH₂, pyruvate, sulfur, mercury, lead, creatinine, cholesterol, α-fetoprotein, chorionic gonadotropin, estrogen, progesterone, testosterone, thyroxine, melatonin, calcitonin, antimullerian hormone, adiponectin, angiotensin, cholecystokinin, corticotrophin-releasing hormone, erythropoietin, bilirubin, creatine, follicle-stimulating hormone, gastrin, ghrelin, glucagon, gonadotropin-releasing hormone, inhibin, growth hormone, growth hormone-releasing hormone, insulin, human placental lactogen, oxytocin, orexin, luteinizing hormone, leptin, prolactin, somatostatin, thrombopoietin, cortisol, aldosterone, estradiol, estriol, estrone, leukotriene, brain natriuretic peptide, neuropeptide Y, histamine, vitamin, mineral, endothelin, renin, enkephalin, DHEA, DHT, alloisoleucine, toxic substance, illegal substance, therapeutic agent, or any metabolite thereof.

In an embodiment 1310, the device further includes at least one memory mechanism for storing instructions for generating and transmitting an electromagnetic control signal. In an embodiment 1320, the device further includes at least one imaging apparatus capable of imaging the approximate quantity within a treatment region of one or more of the at least one artificial antigen presenting cell, or at least one agent thereof. In an embodiment 1330, the device further includes at least one memory location for recording information. In an embodiment 1340, the at least one memory location is configured to record information relating to at least one sensor.

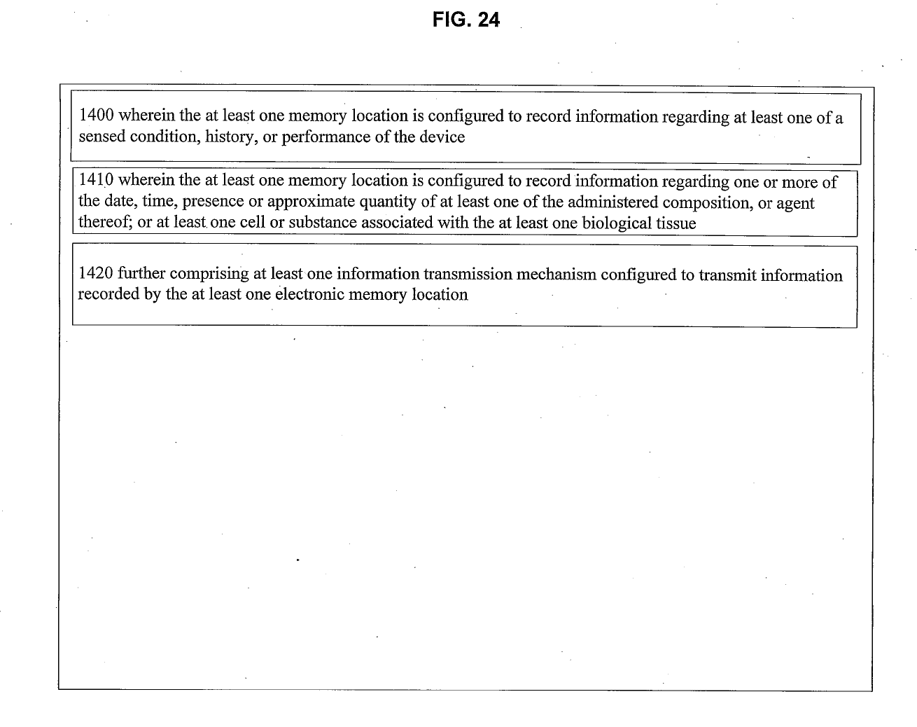

As shown in FIG. 24, in particular embodiments, the device of any of 400, 500, 600, 700, or 800 further include 1400 the at least one memory location is configured to record information regarding at least one of a sensed condition, history, or performance of the device. In an embodiment 1410, the at least one memory location is configured to record information regarding one or more of the date, time, presence or approximate quantity of at least one of the administered composition, or agent thereof; or at least one cell or substance associated with the at least one biological tissue. In an embodiment 1420, the device further includes at least one information transmission mechanism configured to transmit information recorded by the at least one electronic memory location.

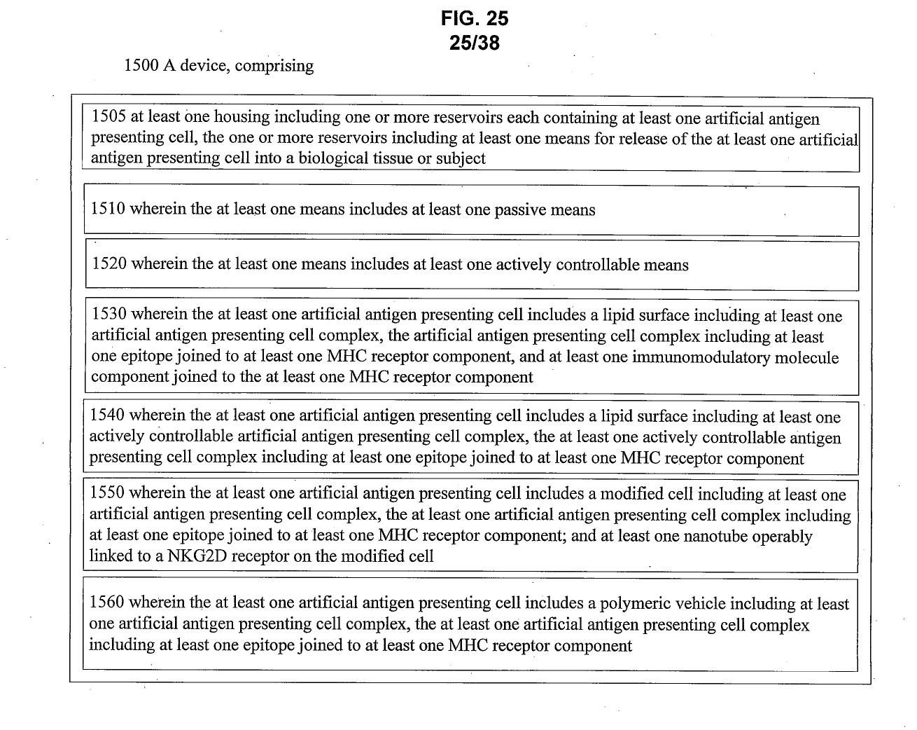

As shown in FIG. 25, in an embodiment, a device 1500 includes 1505 at least one housing including one or more reservoirs each containing at least one artificial antigen presenting cell, the one or more reservoirs including at least one means for release of the at least one artificial antigen presenting cell into a biological tissue or subject. In an embodiment 1510 the at least one means includes at least one passive means. In an embodiment 1520 the at least one means includes at least one actively controllable means. In an embodiment 1530 the at least one artificial antigen presenting cell includes a lipid surface including at least one artificial antigen presenting cell complex, the artificial antigen presenting cell complex including at least one epitope joined to at least one MHC receptor component, and at least one immunomodulatory molecule component joined to the at least one MHC receptor component. In an embodiment 1540, the at least one artificial antigen presenting cell includes a lipid surface including at least one actively controllable artificial antigen presenting cell complex, the at least one actively controllable antigen presenting cell complex including at least one epitope joined to at least one MHC receptor component. In an embodiment 1550, the at least one artificial antigen presenting cell includes a modified cell including at least one artificial antigen presenting cell complex, the at least one artificial antigen presenting cell complex including at least one epitope joined to at least one MHC receptor component; and at least one nanotube operably linked to a NKG2D receptor on the modified cell. In an embodiment 1560, the at least one artificial antigen presenting cell includes a polymeric vehicle including at least one artificial antigen presenting cell complex, the at least one artificial antigen presenting cell complex including at least one epitope joined to at least one MHC receptor component.

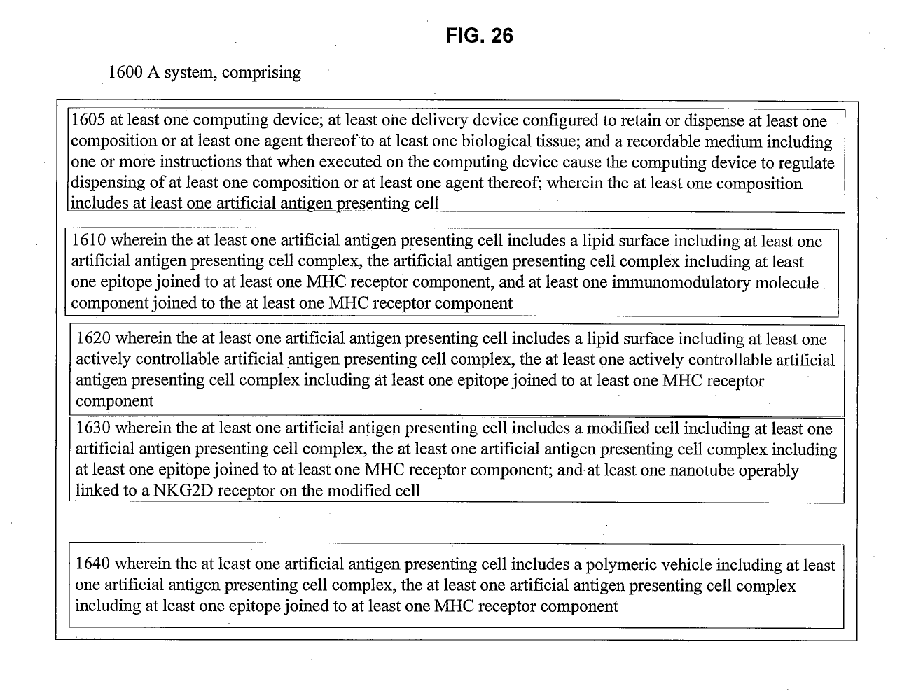

As shown in FIG. 26, in an embodiment, a system 1600 includes 1605 at least one computing device; at least one delivery device configured to retain or dispense at least one composition or at least one agent thereof to at least one biological tissue; and a recordable medium including one or more instructions that when executed on the computing device cause the computing device to regulate dispensing of at least one composition or at least one agent thereof; wherein the at least one composition includes at least one artificial antigen presenting cell. In an embodiment 1610 the at least one artificial antigen presenting cell includes a lipid surface including at least one artificial antigen presenting cell complex, the artificial antigen presenting cell complex including at least one epitope joined to at least one MHC receptor component, and at least one immunomodulatory molecule component joined to the at least one MHC receptor component. In an embodiment 1620 the at least one artificial antigen presenting cell includes a lipid surface including at least one actively controllable artificial antigen presenting cell complex, the at least one actively controllable antigen presenting cell complex including at least one epitope joined to at least one MHC receptor component. In an embodiment 1630 the at least one artificial antigen presenting cell includes a modified cell including at least one artificial antigen presenting cell complex, the at least one artificial antigen presenting cell complex including at least one epitope joined to at least one MHC receptor component; and at least one nanotube operably linked to a NKG2D receptor on the modified cell. In an embodiment, 1640 the at least one artificial antigen presenting cell includes a polymeric vehicle including at least one artificial antigen presenting cell complex, the at least one artificial antigen presenting cell complex including at least one epitope joined to at least one MHC receptor component.

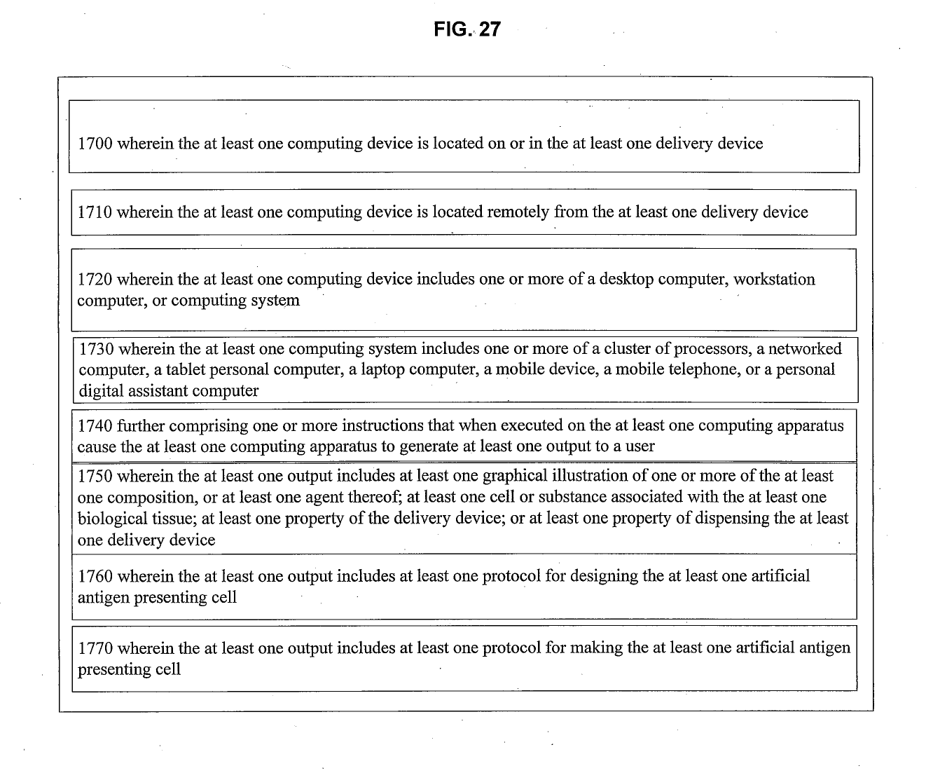

As shown in FIG. 27, in an embodiment, 1700 the at least one computing device is located on or in the at least one delivery device. In an embodiment, 1710 the at least one computing device is located remotely from the at least one delivery device. In an embodiment 1720 the at least one computing device includes one or more of a desktop computer, workstation computer, or computing system. In an embodiment 1730 the at least one computing system includes one or more of a cluster of processors, a networked computer, a tablet personal computer, a laptop computer, a mobile device, a mobile telephone, or a personal digital assistant computer. In an embodiment, 1740 the system further comprises one or more instructions that when executed on the at least one computing apparatus cause the at least one computing apparatus to generate at least one output to a user. In an embodiment, 1750 the at least one output includes at least one graphical illustration of one or more of the at least one composition, or at least one agent thereof; at least one cell or substance associated with the at least one biological tissue; at least one property of the delivery device; or at least one property of dispensing the at least one delivery device. In an embodiment, 1760 the at least one output includes at least one protocol for designing the at least one artificial antigen presenting cell. In an embodiment 1770 the at least one output includes at least one protocol for making the at least one artificial antigen presenting cell.

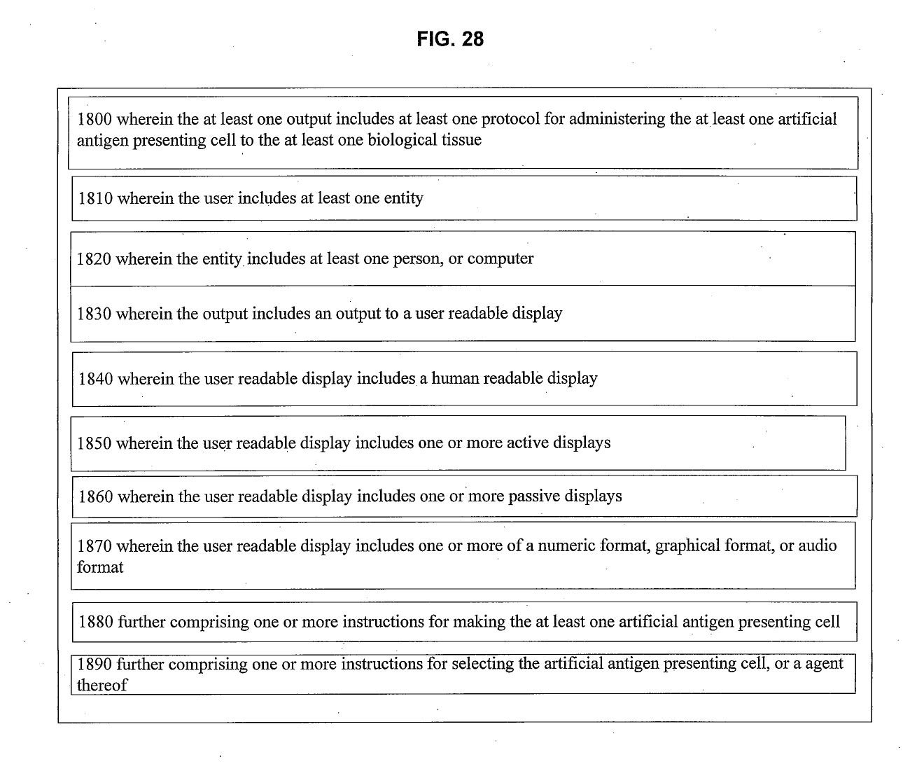

As shown in FIG. 28, in an embodiment 1800 the at least one output includes at least one protocol for administering the at least one artificial antigen presenting cell to the at least one biological tissue. In an embodiment 1810 the user includes at least one entity. In an embodiment 1820 the entity includes at least one person or computer. In an embodiment 1830 the output includes an output to a user readable display. In an embodiment 1840 the user readable display includes a human readable display. In an embodiment 1850 the user readable display includes one or more active displays. In an embodiment 1860 the user readable display includes one or more passive displays. In an embodiment 1870 the user readable display includes one or more of a numeric format, graphical format, or audio format. In an embodiment 1880 the system further comprises one or more instructrions for making the at least one artifician antigen presenting cell. In an embodiment 1890 the system further comprises one or more instructions for selecting the artificial antigen presenting cell or an agent thereof.

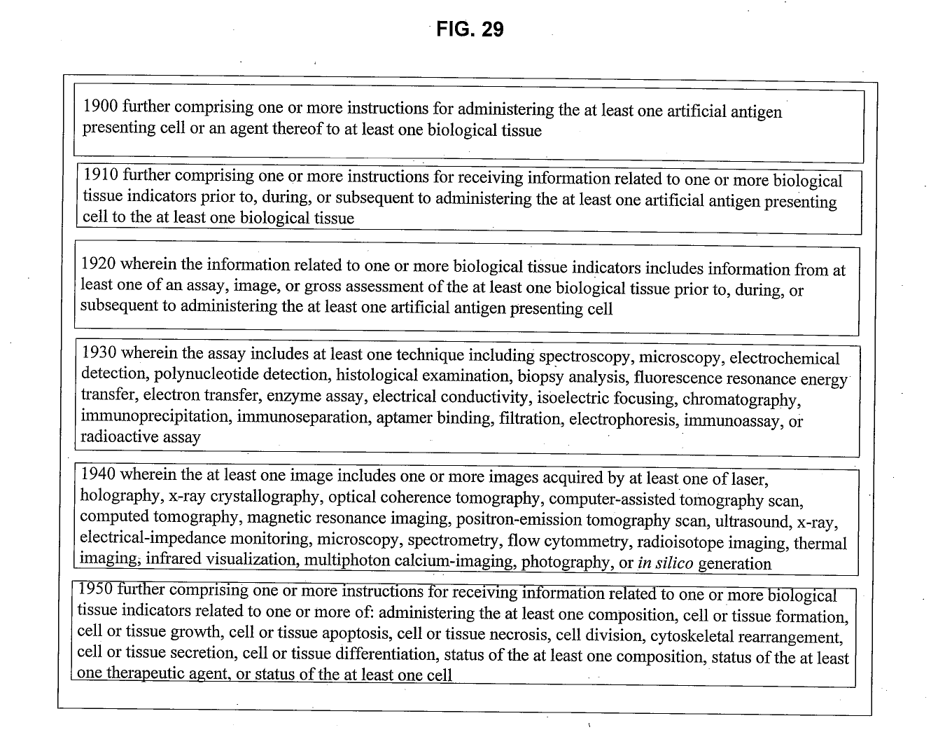

As shown in FIG. 29, in an embodiment 1900 the system further comprises one or more instructions for administering the at least one artificial antigen presenting cell or an agent thereof to at least one biological tissue. In an embodiment 1910, the system further comprises one or more instructions for receiving information related to one or more biological tissue indicators prior to, during, or subsequent to administering the at least one artificial antigen presenting cell to the at least one biological tissue. In an embodiment 1920, the information related to one or more biological tissue indicators includes information from at least one of an assay, image, or gross assessment of the at least one biological tissue prior to, during, or subsequent to administering the at least one artificial antigen presenting cell. In an embodiment 1930, the assay includes at least one technique including spectroscopy, microscopy, electrochemical detection, polynucleotide detection, histological examination, biopsy analysis, fluorescence resonance energy transfer, electron transfer, enzyme assay, electrical conductivity, isoelectric focusing, chromatography, immunoprecipitation, immuno separation, aptamer binding, filtration, electrophoresis, immunoassay, or radioactive assay. In an embodiment, 1940 the at least one image includes one or more images acquired by at least one of laser, holography, x-ray crystallography, optical coherence tomography, computer-assisted tomography scan, computed tomography, magnetic resonance imaging, positron-emission tomography scan, ultrasound, x-ray, electrical-impedance monitoring, microscopy, spectrometry, flow cytommetry, radioisotope imaging, thermal imaging, infrared visualization, multiphoton calcium-imaging, photography, or in silico generation. In an embodiment 1950, the system further comprises one or more instructions for receiving information related to one or more biological tissue indicators related to one or more of: administering the at least one composition, cell or tissue formation, cell or tissue growth, cell or tissue apoptosis, cell or tissue necrosis, cell division, cytoskeletal rearrangement, cell or tissue secretion, cell or tissue differentiation, status of the at least one composition, status of the at least one therapeutic agent, or status of the at least one cell.

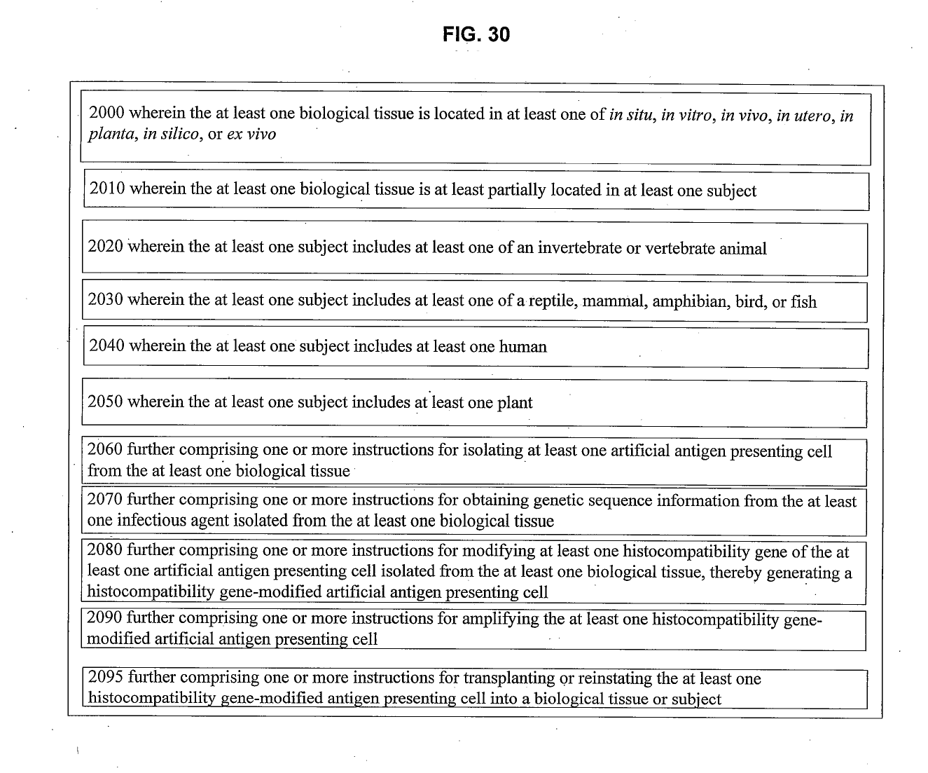

As shown in FIG. 30, in an embodiment 2000 the the at least one biological tissue is located in at least one of in situ, in vitro, in vivo, in utero, in planta, in silico, or ex vivo. In an embodiment 2010 the at least one biological tissue is at least partially located in at least one subject. In an embodiment 2020 the at least one subject includes at least one of an invertebrate or vertebrate animal. In an embodiment 2030 the at least one subject includes at least one of a reptile, mammal, amphibian, bird, or fish. In an embodiment 2040 the at least one subject includes at least one human. In an embodiment 2050, the at least one subject includes at least one plant. In an embodiment 2060, the system further comprises one or more instructions for isolating at least one artificial antigen presenting cell from the at least one biological tissue. In an embodiment 2070, the system further comprises one or more instructions for obtaining genetic sequence information from the at least one infectious agent isolated from the at least one biological tissue. In an embodiment 2080, the system further comprises one or more instructions for modifying at least one histocompatibility gene of the at least one artificial antigen presenting cell isolated from the at least one biological tissue, thereby generating a histocompatibility gene-modified artificial antigen presenting cell. In an embodiment 2090, the system further comprises one or more instructions for amplifying the at least one histocompatibility gene-modified artificial antigen presenting cell. In an embodiment 2095, the system further comprises one or more instructions for transplanting or reinstating the at least one histocompatibility gene-modified antigen presenting cell into a biological tissue or subject.

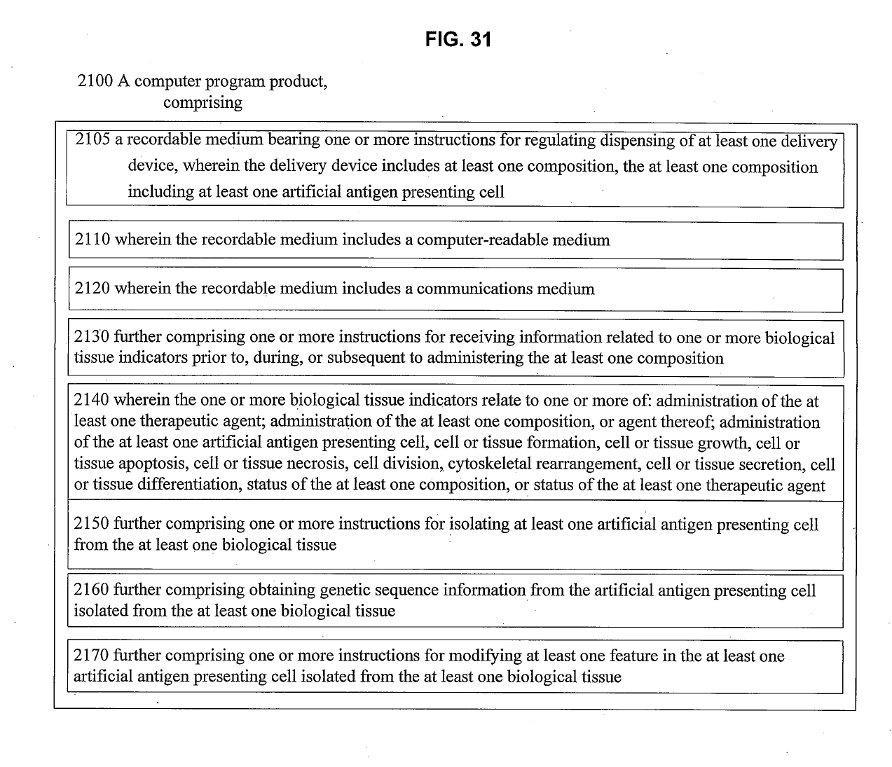

As shown in FIG. 31, a computer program product 2100, includes 2105 a recordable medium bearing one or more instructions for regulating dispensing of at least one delivery device, wherein the delivery device includes at least one composition, the at least one composition including at least one artificial antigen presenting cell. In an embodiment 2110 the recordable medium includes a computer-readable medium. In an embodiment 2120, the recordable medium includes a communications medium. In an embodiment 2130, the computer program product further rincludes one or more instructions for receiving information related to one or more biological tissue indicators prior to, during, or subsequent to administering the at least one composition. In an embodiment 2140, the one or more biological tissue indicators relate to one or more of: administration of the at least one therapeutic agent; administration of the at least one composition, or agent thereof; administration of the at least one artificial antigen presenting cell, cell or tissue formation, cell or tissue growth, cell or tissue apoptosis, cell or tissue necrosis, cell division, cytoskeletal rearrangement, cell or tissue secretion, cell or tissue differentiation, status of the at least one composition, or status of the at least one therapeutic agent. In an embodiment 2150, the computer program product further includes one or more instructions for isolating at least one artificial antigen presenting cell from the at least one biological tissue. In an embodiment 2160, the computer program product further comprises obtaining genetic sequence information from the artificial antigen presenting cell isolated from the at least one biological tissue. In an embodiment 2170 the computer program product further comprises one or more instructions for modifying at least one feature in the at least one artificial antigen presenting cell isolated from the at least one biological tissue.

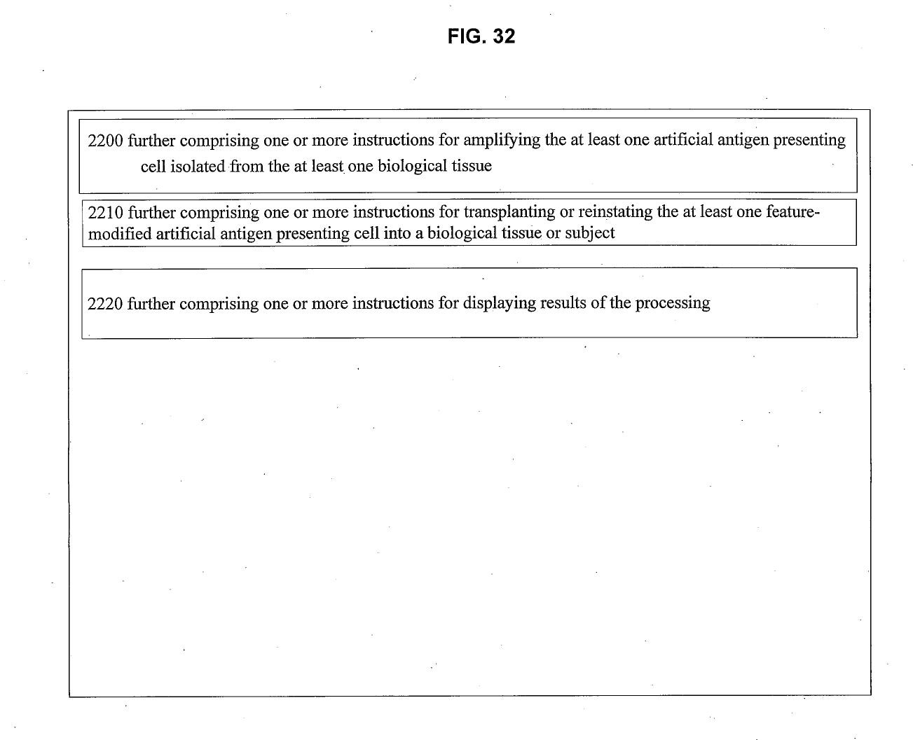

As shown in FIG. 32, in an embodiment 2200, the computer program product further comprises one or more instructions for amplifying the at least one artificial antigen presenting cell isolated from the at least one biological tissue. In an embodiment 2210, the computer program product further comprises one or more instructions for transplanting or reinstating the at least one feature-modified artificial antigen presenting cell into a biological tissue or subject. In an embodiment 2220 the computer program product further comprises one or more instructions for displaying results of the processing.

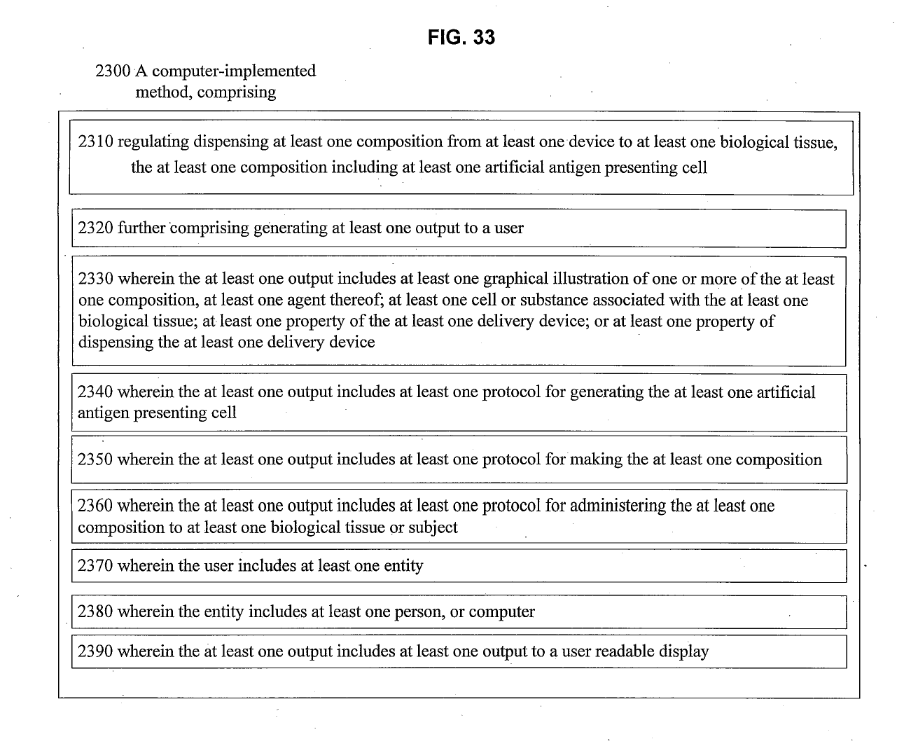

As shown in FIG. 33, a computer-implemented method 2300 includes in an embodiment 2310, one or more instructions for regulating dispensing at least one composition from at least one device to at least one biological tissue, the at least one composition including at least one artificial antigen presenting cell. In an embodiment 2320 the computer-implemented method further includes generating at least one output to a user. In an embodiment 2330 the at least one output includes at least one graphical illustration of one or more of the at least one composition, or at least one agent thereof; at least one cell or substance associated with the at least one biological tissue; at least one property of the at least one delivery device; or at least one property of dispensing the at least one delivery device. In an embodiment 2340 the at least one output includes at least one protocol for generating the at least one artificial antigen presenting cell. In an embodiment 2350 the at least one output includes at least one protocol for making the at least one composition. In an embodiment 2360 the at least one output includes at least one protocol for administering the at least one composition to at least one biological tissue or subject. In an embodiment 2370 the user includes at least one entity. In an embodiment 2380 the entity includes at least one person, or computer. In an embodiment 2390 the at least one output includes at least one output to a user readable display.

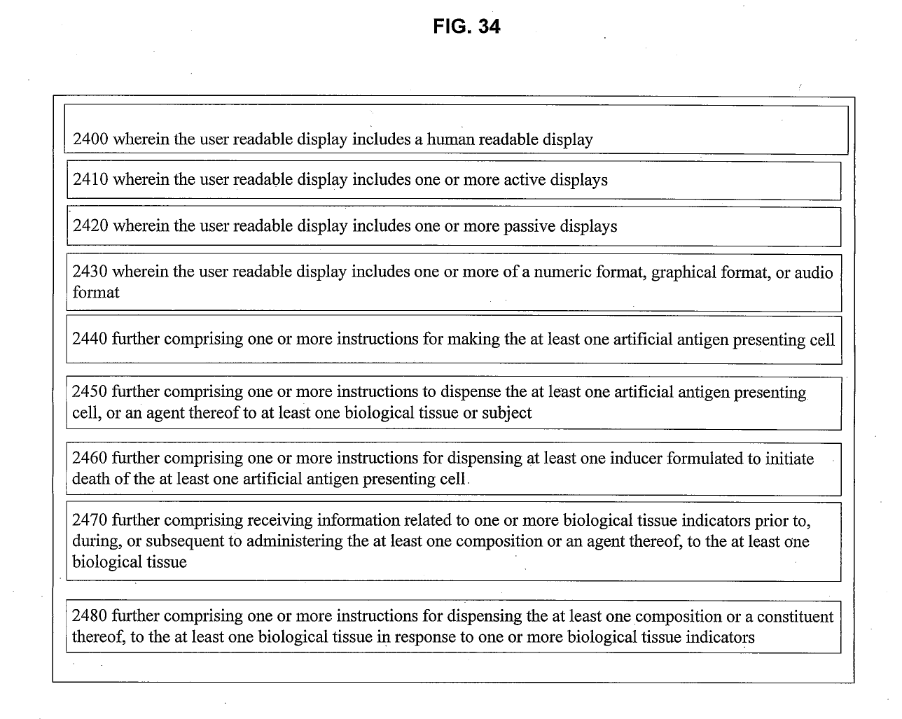

As shown in FIG. 34, in an embodiment 2400 the user readable display includes a human readable display. In an embodiment 2410 the user readable display includes one or more active displays. In an embodiment 2420, the user readable display includes one or more passive displays. In an embodiment 2430, the user readable display includes one or more of a numeric format, graphical format, or audio format. In an embodiment 2440, the computer-implemented method further comprises one or more instructions for making the at least one artificial antigen presenting cell. In an embodiment 2450, the computer-implemented method further comprises one or more instructions to dispense the at least one artificial antigen presenting cell, or an agent thereof to at least one biological tissue or subject. In an embodiment 2460 the computer-implemented method further comprises one or more instructions for dispensing at least one inducer formulated to initiate death of the at least one artificial antigen presenting cell. In an embodiment 2470 the computer-implemented method further comprises receiving information related to one or more biological tissue indicators prior to, during, or subsequent to administering the at least one composition or an agent thereof, to the at least one biological tissue. In an embodiment 2480, the computer-implemented method further comprises one or more instructions for dispensing the at least one composition or an agent thereof, to the at least one biological tissue in response to one or more biological tissue indicators.

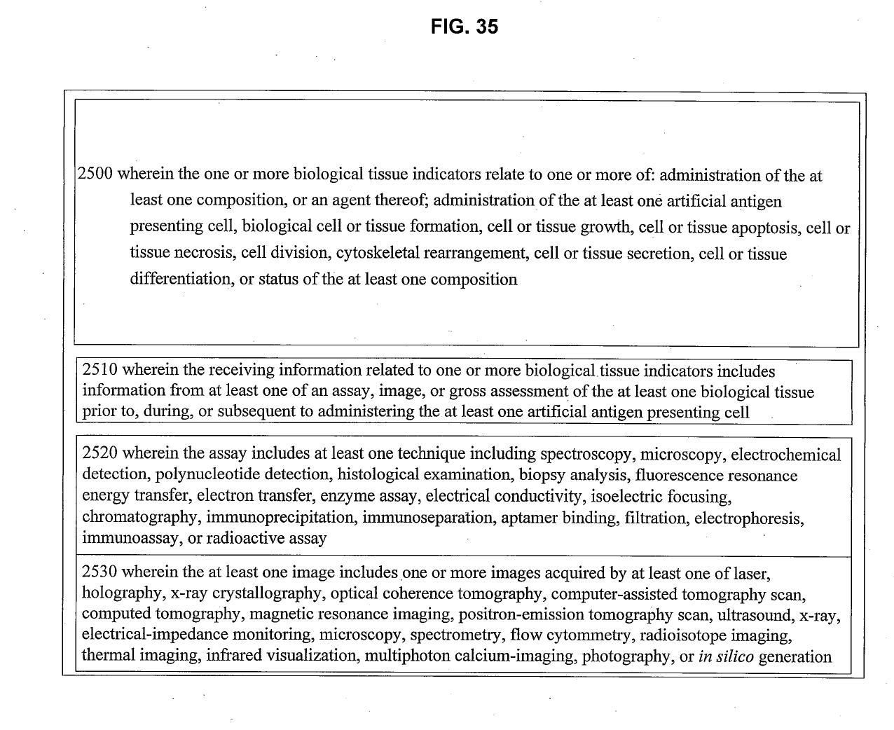

As shown in FIG. 35, in an embodiment 2500, the one or more biological tissue indicators relate to one or more of: administration of the at least one composition, or agent thereof; administration of the at least one artificial antigen presenting cell, biological cell or tissue formation, cell or tissue growth, cell or tissue apoptosis, cell or tissue necrosis, cell division, cytoskeletal rearrangement, cell or tissue secretion, cell or tissue differentiation, or status of the at least one composition. In an embodiment 2510, the receiving information related to one or more biological tissue indicators includes information from at least one of an assay, image, or gross assessment of the at least one biological tissue prior to, during, or subsequent to administering the at least one artificial antigen presenting cell. In an embodiment 2520, the assay includes at least one technique including spectroscopy, microscopy, electrochemical detection, polynucleotide detection, histological examination, biopsy analysis, fluorescence resonance energy transfer, electron transfer, enzyme assay, electrical conductivity, isoelectric focusing, chromatography, immunoprecipitation, immuno separation, aptamer binding, filtration, electrophoresis, immunoassay, or radioactive assay. In an embodiment 2530 the at least one image includes one or more images acquired by at least one of laser, holography, x-ray crystallography, optical coherence tomography, computer-assisted tomography scan, computed tomography, magnetic resonance imaging, positron-emission tomography scan, ultrasound, x-ray, electrical-impedance monitoring, microscopy, spectrometry, flow cytommetry, radioisotope imaging, thermal imaging, infrared visualization, multiphoton calcium-imaging, photography, or in silico generation.

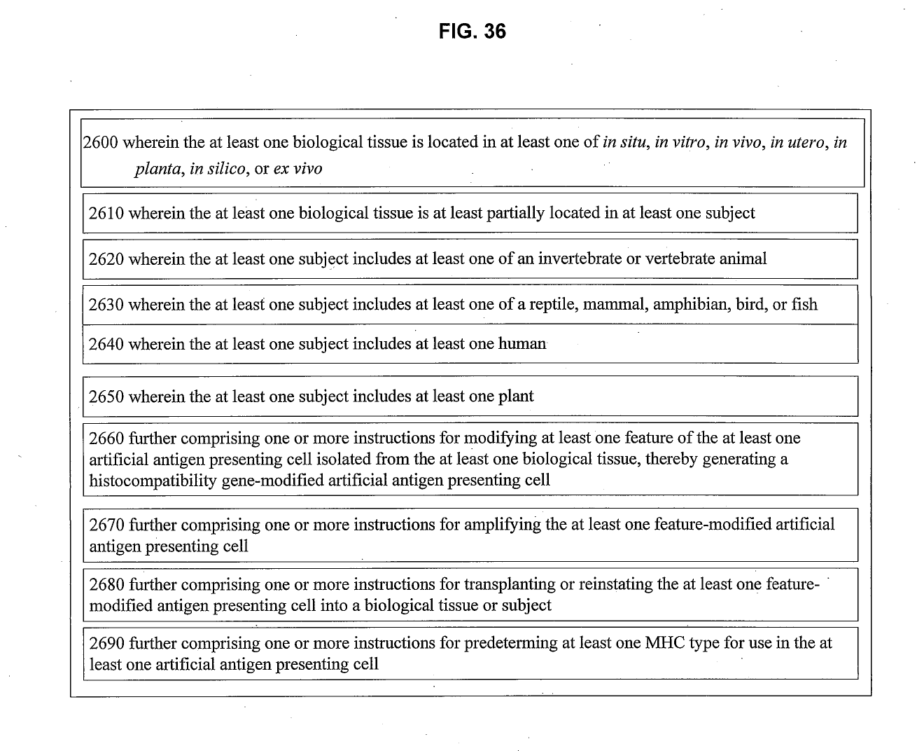

As shown in FIG. 36, in an embodiment 2600 wherein the at least one biological tissue is located in at least one of in situ, in vitro, in vivo, in utero, in planta, in silico, or ex vivo. In an embodiment 2610, the at least one biological tissue is at least partially located in at least one subject. In an embodiment 2620, the at least one subject includes at least one of an invertebrate or vertebrate animal. In an embodiment 2630, the at least one subject includes at least one of a reptile, mammal, amphibian, bird, or fish. In an embodiment 2640, the at least one subject includes at least one human. In an embodiment 2650, the at least one subject includes at least one plant. In an embodiment 2660, the at least one computer-implemented method further comprises one or more instructions for modifying at least one feature of the at least one artificial antigen presenting cell isolated from the at least one biological tissue, thereby generating a histocompatibility gene-modified artificial antigen presenting cell. In an embodiment 2670 the computer-implemented method further comprises one or more instructions for amplifying the at least one feature-modified artificial antigen presenting cell. In an embodiment 2680 the computer-implemented method further comprises one or more instructions for transplanting or reinstating the at least one feature-modified antigen presenting cell into a biological tissue or subject. In an embodiment 2690 the computer-implemented method further comprises one or more instructions for predeterming at least one MHC type for use in the at least one artificial antigen presenting cell.

In an embodiment, the artificial antigen presenting cells described herein are useful for isolating, identifying, locating, modifying, or expanding T cell populations. In an embodiment, the artificial antigen presenting cells described herein are useful for vaccinating a subject. In an embodiment, the artificial antigen presenting cells described herein are useful for modulating immune responses in at least one of in vivo, in vitro, in situ, ex vivo, in utero, in silico, or in planta.

In an embodiment, a method of modulating at least one T cell responsive to an epitope includes providing an effective amount of at least one composition including an artificial antigen presenting cell described herein to a biological tissue or fluid including at least one T cell. In an embodiment, the composition further includes at least one detection material. In an embodiment, the method further includes detecting at least one T cell bound to the at least one composition including at least one artificial antigen presenting cell. In an embodiment, the method further includes isolating the bound T cells, opitionally locating the bound T cells, optionally quantifying the bound T cells, optionally expanding the bound T cells, optionally stimulating the bound T cells, opitionally genetically altering the bound T cells, or optionally implanting or transplanting the bound T cells.

In an embodiment, a method of increasing immunological tolerance, or decreasing rejection of at least one biological tissue transplant in a subject includes providing at least one composition including at least one artificial antigen presenting cell described herein to at least one biological tissue or at least one subject, and transplanting the at least one biological tissue to the subject. In an embodiment, the at least one composition is sufficient to induce anergy in at least one T cell of at least one of the biological tissue or the subject.

In an embodiment, a method of modulating an immune response includes providing to at least one biological tissue, at least one artificial antigen presenting cell effective to modulate an immune response, wherein the at least one artificial antigen presenting cell includes at least one composition described herein. In an embodiment, the at least one biological tissue is located in a subject. In an embodiment, the at least one biological tissue include at least one cell of skin, brain, lung, liver, spleen, bone marrow, thymus, germinal center, heart, myocardium, endocardium, pericardium, lymph node, bone, cartilage, pancreas, kidney, gall bladder, stomach, duct, valve, smooth muscle, appendix, intestine, testes, uterus, rectum, nervous system, blood, lymph, eye, scalp, nail bed, ear, ovary, oviduct, tongue, tonsil, adenoid, liver, blood vessel, breast, bladder, urethra, ureter, prostate, vas deferens, fallopian tubes, esophagus, oral cavity, nasal cavity, otic cavity, connective tissue, muscle tissue, mucosa-associated lymphoid tissue (MALT), placental tissue, fetal tissue, malignant tissue, large intestine, small intestine, spinal fluid, spine, mucosal tissue, or adipose tissue. In an embodiment, the at least one biological tissue includes one or more of a stalk, stem, leaf, root, plant, or tendril. In an embodiment, the at least one biological tissue includes at least one cell mass or wound.

In an embodiment, the method further includes inducing the at least one artificial antigen presenting cell to dissociate. In an embodiment, inducing the at least one artificial antigen presenting cell to dissociate includes exposing the artificial antigen presenting cell to at least one of energy, magnetism, pH, exposure to at least one adverse agent, exposure to an electric field, or exposure to at least one therapeutic agent. In an embodiment, the at least one adverse agent includes at least one of a pollutant, warfare agent, histamine, toxin, or the like. In an embodiment, the energy includes at least one of ultrasound energy, infrared energy, electromagnetic energy, thermal energy, x-ray energy, or shock-wave energy. In an embodiment, the artificial antigen presenting cell is provided by way of a device. In an embodiment, the device includes at least one solid surface. In an embodiment, the device includes at least one of a column, syringe, array, tube, dish, flask, implant, microelectromechanical device, nanoelectromechanical device, or other device.

In an embodiment, the method further includes inducing the release or exposure of at least one therapeutic agent from the antigen presenting cell. In an embodiment, the at least one therapeutic agent is formulated to modulate at least one of the viability, proliferation, or metastasis of at least one tumor cell. In an embodiment, the at least one therapeutic agent is formulated to induce apoptosis in one or more biological cells of a biological tissue. In an embodiment, the at least one artificial antigen presenting cell is provided in an effective amount in relation to at least one of inflammation, infection, immunosuppression, cancer, allergy, asthma, lactose intolerance, atherosclerosis, diarrhea, fever, autoimmune disease, diabetes, arthritis, wound healing, exposure to an environmental agent, or dental caries. In an embodiment, the environmental agent includes at least one of a pollutant, chemical warfare agent, allergen, temperature, sunlight, ultraviolet radiation, or other environmental agent. In an embodiment, the infection includes at least one of vaginal infection, oral infection, dental infection, urogenital infection, ear infection, eye infection, tonsillitis, ulcer, intestinal blockage or infection, skin infection, nail infection, sinus infection, urinary tract infection, kidney infection, bioweapon infection, pharyngitis, or laryngitis.

As described herein, in an embodiment, the biological tissue is located in a subject. In an embodiment, the subject includes at least one vertebrate or invertebrate animanl. In an embodiment, the subject includes at least one of a reptile, mammal, amphibian, bird, or fish. In an embodiment, the subject includes a human. In an embodiment, the subject tincludes at least one of a plant or alga. In an embodiment, the subject includes subject includes at least one of a sheep, goat, frog, dog, cat, rat, mouse, vermin, rodent, reptile, monkey, horse, cow, pig, chicken, fowl, shellfish, fish, turkey, llama, alpaca, bison, buffalo, ape, primate, ferret, wolf, fox, coyote, deer, rabbit, guinea pig, yak, elephant, tiger, lion, cougar, chinchilla, mink, reindeer, elk, camel, fox, elk, deer, ruminant, canid, felid, lagomorphs, raccoon, donkey, or mule.

As disclosed herein, in an embodiment the artificial antigen presenting cell is formulated to modulate at least one immune response. In an embodiment, the immune response includes at least one of an allergic or autoimmune response. In an embodiment, the immune response includes at least one lymphocyte response. In an embodiment, the immune response includes at least one of modulation of immune cell activation, modulation of immune cell anergy, modulation of immune cell antibody production, modulation of immune cell death, modulation of immune cell class or subclass, modulation of immune cell type, or modulation of production of at least one cytokine.

In an embodiment, the method of administering at least one composition described herein includes selecting for administration an amount or type of composition for administration.

For example, in an embodiment a T cell population possessing specific reactivity to a particular epitope or antigen is able to be isolated, and optionally manipulated (e.g., expanded, primed, stimulated, etc.) ex vivo or in vivo. In an embodiment, the T cell population includes at least one population that specifically reacts to at least one antigen related to one or more of an autoimmune disease or disorder; cancer; allergy; asthma, infection; or other particular antigen or epitope.

In an embodiment, a method of vaccinating a subject includes providing an effective amount of a composition including at least one artificial antigen presenting cell described herein, to at least one biological tissue.

In an embodiment, the artificial antigen presenting cells described herein are useful for quantifying or identifying a T cell population specific for a particular antigen.

In an embodiment, the artificial antigen presenting cells described herein are useful for predicting a MHC that will be compatible between a donor and recipient; or for. predicting what MHC:epitope combination will be most efficient for eliciting an immune response in a particular subject or biological tissue. For example, binding of peptides to MHC molecules is allele specific. Sequence requirements for binding can be defined by testing sets of peptides with the capability to bind to a given MHC molecule. Such characterization studies have been performed with various MHC molecules, the motifs of which have served to predict antigenic peptides along a protein sequence. Based on the MHC contact residues, algorithms have been developed to predict MHC-binding, and several databases include this information. For example, prediction of MHC:epitopes has been made by using machine learning approaches such as artificial neural networks, or hidden Markov models. See, for example, Schueler-Furman, et al., Prot. Sci., vol. 9:1838-1846 (2000); Donnes and Elofsson, BMC Bioinform. Vol. 3(25): 1-8 (2002); the worldwide web at anthonynolan.org.uk/HIG (HLA sequence database); ebi.ac.uk/imgt (MHC, TCR 7 sequences); ashi-hla.org (sequences and frequencies of MHC alleles); Nuc. Acids Res. vol. 31 (13):3621-3624 (2003); each of which is incorporated herein by reference. For example, general information is attainable by testing a data set of known epitopes that bind, compared with known epitopes that do not bind the MHC. This set is used to build a model that can discriminate between epitopes that bind and epitopes that do not bind. The model can then be sued to predict whether a novel peptide can Thus, prediction of MHC:epitopes can be predicted based on structure, sequence, or both.

In an embodiment, the at least one antigen presenting cell complex includes at least two different epitopes joined to the at least one MEIC receptor component. In an embodiment, the at least one MHC receptor component is customized for at least one subject or at least one group of subjects (for example, by utilizing the database information described herein, or other genetic or proteomic information). In an embodiment, the at least one MEC receptor component shares at least one allele with the subject's endogenous MHC. In an embodiment, the at least one antigen presenting cell complex is customized for at least one subject or at least one group of subjects. In an embodiment, the lipid surface includes two or more antigen presenting cell complexes, each complex including at least one epitope joined to at least one MHC receptor component, wherein at least two of the epitopes are different epitopes of the same antigen. In an embodiment, the composition further comprises at least two antigen presenting cell complexes, each complex including at least one different mode of joining. In an embodiment, at least two of the components of the antigen presenting cell complex are displayed in a pre-determined arrangement. In an embodiment, the at least one pre-determined arrangement is determinable by one or more desired immune responses. In an embodiment, the at least one pre-determined arrangement includes a pre-determined spatial arrangement. In an embodiment, the at least one pre-determined arrangement includes a pre-determined temporal sequence.

In an embodiment, the present disclosure relates to methods including predicting a useful MHC (for example based on donor or recipient MHC), testing the predicted MHC epitopes (by way of algorithm or actual T cell recognition), and using the epitopes that were determined to be useful in an artificial antigen presenting cell. For example, in an embodiment, a method herein provides for determining an effective MHC:epitope compatibility between donor and recipient, based on antigens from the donor or recipient. In an embodiment, a method herein provides for determining an effective MHC: epitope combination for eliciting an immune response in a subject (e.g. vaccination, control of autoimmune disease, or other reaction). For example, in an embodiment, MHC:epitope combinations identified are utilized directly as a vaccine to the combinations that have sequence recognition with pathogen-derived peptides.

As disclosed herein, in an embodiment, the MHC:epitope combinations are utilized in conjunction with at least one accessory molecule, or at least one co-stimulatory molecule on the artificial antigen presenting cell. Also, as disclosed herein, in an embodiment, the artificial antigen presenting cell(s) are manipulated (e.g., stimulated, expanded, activated, etc.) ex vivo, in vitro, or in vivo.

In an embodiment, the pre-determined arrangement includes a pre-determined spatial arrangement. In an embodiment, the pre-determined arrangement includes a pre-determined temporal sequence. For example, the spacing, size, and geometric arrangement varies, depending on at least one of the desired molecular components in the artificial antigen presenting cell complex, the desired immune response, or the desired administration protocol. For example, in an embodiment, the spacing among the artificial antigen presenting cell complex components includes about 0.1 micron, about 0.2 microns, about 0.3 microns, about 0.4 microns, about 0.5 microns, about 0.6 microns, about 0.7 microns, about 0.8 microns, about 0.9 microns, about 1 micron, about 2 microns, about 3 microns, about 4 microns, about 5 microns, about 6 microns, about 7 microns, about 8 microns, about 9 microns, about 10 microns, about 15 microns, about 20 microns, about 25 microns, about 30 microns, about 35 microns, about 40 microns, about 50 microns, or any value less than or therebetween. Likewise, the spacing between two artificial antigen presenting cell complexes (e.g., a suite of artificial antigen presenting cell complexes) includes about 0.1 micron, about 0.2 microns, about 0.3 microns, about 0.4 microns, about 0.5 microns, about 0.6 microns, about 0.7 microns, about 0.8 microns, about 0.9 microns, about 1 micron, about 2 microns, about 3 microns, about 4 microns, about 5 microns, about 6 microns, about 7 microns, about 8 microns, about 9 microns, about 10 microns, about 15 microns, about 20 microns, about 25 microns, about 30 microns, about 35 microns, about 40 microns, about 50 microns, or any value less than or therebetween. See, for example, U.S. Patent App. Pub. No. 20080317724 A1, which is incorporated herein by reference.

In an embodiment, the MHC receptor component includes at least a portion of one or more of a Major Histocompatibility Class I protein, Major Histocompatibility Class II protein, or Major Histocompatibility Class III protein. As described herein, the MHC includes a gene cluster, as well as associated genes and their gene products (for example, proteins utilized in MHC receptor function). In an embodiment, the at least one MHC receptor component includes at least a portion of one or more of β-2 microglobulin, Transporter Associated with Antigen Processing (TAP), MHC class I α-1 domain, MHC class I α-2 domain, MHC class I α-3 domain, tapasin, calreticulum, ERP57, or calnexin. In an embodiment, the at least one MHC receptor component includes at least one of human leukocyte antigen (HLA), H-Y, H-2, dog leukocyte antigen (DLA), bovine leukocyte antigen (BOLA), equine leukocyte antigen (ELA), swine leukocyte antigen (SLA), Rhesus monkey leukocyte antigen (RhL-A), B-locus (chicken), feline leukocyte antigen (FLA), or chimpanzee leukocyte antigen (ChL-A). In an embodiment, the at least one MHC receptor component includes at least a portion of one or more of a MHC class II α domain, or a β domain. In an embodiment, the at least a portion of the MHC class II α domain includes at least a portion of one or more of α-1 domain or α-2 domain. In an embodiment, the at least a portion of the β domain includes at least a portion one of one or more of β-1 domain or β-2 domain. In an embodiment, the at least one MHC receptor component encodes at least one gene product of one or more of HLA-A, HLA-B, HLA-C, HLA-DPA1, HLA-DPB1, HLA-DRA, HLA-DRB1, HLA-DQA1, or HLA-DQB1 genes.

A. Excitation of the Photo Activatable Molecule

In an embodiment, the one or more methods optionally include providing electromagnetic energy to the subject, where the electromagnetic energy is configured to induce a response from the photoactivatable molecules associated with the modified red blood cells. In illustrative embodiments, excitation of the one or more photoactivatable molecules directly and/or indirectly damages the target cell and/or the red blood cell, or other vehicle. For example, rupturing the vehicle allows for release or exposure of the artificial antigen presenting cell, in an embodiment.

In illustrative embodiments, the electromagnetic energy includes, but is not limited to, one or more frequencies having one or more characteristics that taken as a whole are not considered unduly harmful to the subject. In illustrative examples, such electromagnetic energy may include frequencies optionally including visible light (detected by the human eye between approximately 400 nm and 700 nm) as well as infrared (longer than 700 nm) and limited spectral regions of ultraviolet light, such as UVA light (between approximately 320 nm and 400 nm). Electromagnetic energy includes, but is not limited to, single photon electromagnetic energy, two photon electromagnetic energy, multiple wavelength electromagnetic energy, and extended-spectrum electromagnetic energy.

Electromagnetic energy may be configured as a continuous beam or as a train of short pulses. In the continuous wave mode of operation, the output is relatively consistent with respect to time. In the pulsed mode of operation, the output varies with respect to time, optionally having alternating “on” and “off” periods. Electromagnetic energy may be provided by one or more lasers, for example, having one or more of a continuous or pulsed mode of action. One or more pulsed lasers may include, but are not limited to, Q-switched lasers, mode locking lasers, and pulsed-pumping lasers. Mode locked lasers emit extremely short pulses on the order of tens of picoseconds down to less than 10 femtoseconds, the pulses optionally separated by the time that a pulse takes to complete one round trip in the resonator cavity. Due to the Fourier limit, a pulse of such short temporal length may have a spectrum which contains a wide range of wavelengths.

In an embodiment, the electromagnetic energy is focused at a depth of approximately 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, 2.5 mm, 2.6 mm, 2.7 mm, 2.8 mm, 2.9 mm, or 3.0 mm below the surface of the skin, beyond the surface of a wall of a blood vessel (e.g., in the blood vessel lumen), and/or beyond a surface of an internal location. In an embodiment, the electromagnetic energy is focused at a depth of approximately 0.1 to 3 mm, 0.1 to 2.5 mm, 0.1 to 2.0 mm, 0.1 to 1.5 mm, 0.1 to 1.0 mm, 0.1 to 0.5 mm, 0.5 to 3.0 mm, 0.5 to 2.5 mm, 0.5 to 2.0 mm, 0.5 to 1.5 mm, 0.5 to 1.0 mm, 1.0 to 3.0 mm, 1.0 to 2.5 mm, 1.0 to 2.0 mm, 1.0 to 1.5 mm, 1.5 to 3.0 mm, 1.5 to 2.5 mm, 1.5 to 2.0 mm, 2.0 to 3.0 mm, 2.0 to 2.5 mm, or 2.5 to 3.0 mm below the surface of the skin, beyond the surface of a wall of a blood vessel (e.g., in the blood vessel lumen), and/or beyond a surface of an internal location.

In an embodiment, the electromagnetic energy is generated by two photons having the same wavelength or substantially the same wavelength. In an embodiment, the electromagnetic energy is generated by sets of two photons having different wavelengths. Electromagnetic energy at the energy levels of the two photons is optionally focused at a depth below the surface of the skin, beyond the surface of a wall of a blood vessel (e.g., in the blood vessel lumen), and/or beyond a surface of an internal location, and/or optionally at one or more depths. As used herein, the term “two-photon” may include excitation optionally using one or more femtosecond lasers. In an embodiment, two photon electromagnetic energy is coupled through a virtual energy level and/or coupled through an intermediate energy level.

As used herein, the term “extended-spectrum” may include a range of possible electromagnetic radiation wavelengths within the full spectrum of possible wavelengths, optionally from extremely long to extremely short and optionally including wide spectrum and narrow spectrum wavelengths.

In an embodiment, the electromagnetic energy may be defined spatially and/or directionally. In an embodiment, the electromagnetic energy may be spatially limited, optionally spatially focused and/or spatially collimated. In an embodiment, the electromagnetic energy may be directionally limited, directionally varied, and/or directionally variable. In illustrative embodiments, the electromagnetic energy optionally contacts less than an entire possible area, or less than an entire possible target, and/or is limited to a certain depth within a tissue. In illustrative embodiments, the electromagnetic energy is spatially and/or directionally limited so that only areas approximately bounded by the walls of one or more blood vessels are provided with electromagnetic energy. In illustrative embodiments, the electromagnetic energy may be provided over an entire field (e.g., scanning across and/or the length of a blood vessel lumen), through movement of the electromagnetic source, and/or through illumination from more than one, two, three, four, and/or multiple sources in the device. Alternatively, in some approaches illumination may be provided over less than an entire field, for example, by illuminating according to a vector scanning approach. In such approaches, illumination energy may be directed to less than all of the area, e.g., primarily in and/or around vascular regions or in areas of interest, such as areas where blood components of interest may be suspected to be or predicted to be. Alternatively, such illumination of less than the entire region may be implemented by a scanning pattern encompassing the entire region combined with activating the source of electromagnetic energy only in selected locations.

B. Methods for Disrupting Modified Red Blood Cells Loaded with Molecular Agents

A modified red blood cell loaded with a molecular agent (e.g., a therapeutic agent) may be targeted to a specific pathogen or cell using the methods described herein. Upon interacting with the target cell, the modified red blood cell may be induced to release the therapeutic agent. There are a number of methods described for controlled release of a therapeutic agent from a red blood cell such as, for example, normal red blood cell break down, accelerated red blood cell breakdown due, for example, to incompatible cells from different individuals or species, inappropriate blood type, and/or introduction of immunogenic protein on the surface of the red blood cell, administering energy to selectively disrupt red blood cells such as, for example, ultrasound, radiofrequency, microwave, and/or infrared, incorporation of an enzyme that digests the cell membrane from the inside out, addition of a second agent added at a specific time the initiates cell breakdown, and use of the complement system (See, e.g., U.S. Patent Application 2007/0243137 A1, which is incorporated herein by reference).

In some instances, it may be of benefit to target macrophages with the modified red blood cells. Under normal circumstances, aged and/or damaged red blood cells are cleared from circulation by macrophage phagocytosis. This process may be enhanced by artificially clustering the modified red blood cell transmembrane proteins using, for example, ZnCl₂ and bissulfosuccinimideilsuberate (BS³; See, e.g., U.S. Pat. No. 6,139,836, which is incorporated herein by reference). In this instance, the modified red blood cells are treated with 1 mM ZnCl₂ in saline solution to cluster the proteins and subsequently treated with BS³ for 15 min to irreversibly cross-link the clustered proteins.

A modified red blood cell may be loaded with metal particles which upon interaction with an external energy source preferentially causes the modified red blood cells to be disrupted (See, e.g., U.S. Pat. No. 6,645,464, which is incorporated herein by reference). As such, modified red blood cells may be loaded with colloidal gold or gold clusters (1-20 nm in size) using hypotonic lysis in 5 mM phosphate buffer (pH 8) with 10 μM magnesium sulfate at a temperature of 4° C. In some instances, the modified red blood cells may be simultaneously loaded with metal particles and a therapeutic agent, for example. The cells are resealed by warming to 37° C. for 5-15 min in the presence of 0.2 M NaCl, for example. In some instances, it may be beneficial to add small nucleating metal particles to a modified red blood cell and subsequently enlarging the particles in situ (See, e.g., U.S. Pat. No. 6,645,464, which is incorporated herein by reference). As such, modified red blood cells that have been loaded with small nucleating metal particles and resealed may be further treated with an autometallographic developer solution containing gold ions, for example, to generate large internal metal particles. The modified red blood cells may be targeted to a specific tissue bed such as, for example, a tumor, and subsequently irradiated to disrupt the modified red blood cell and release the loaded therapeutic agent.

C. Monitoring Interaction of Modified Red Blood Cell with Target Molecule

In an embodiment, it may be useful to monitor the interaction of the modified red blood cells with a target molecule or target cell prior to the exposure of a subject to light of a suitable wavelength. The interaction may be monitored by providing a modified red blood cell which includes a signaling molecule that is detectable upon binding of the modified red blood cell to the target cell or molecule.

In an embodiment, red blood cells may be modified with an aptamer-based molecular beacon to detect interaction of the modified red blood cell with a target cell. RNA or DNA oligonucleotide-based aptamers in combination with fluorescent tags, for example, may be used as molecular beacons to detect interactions between a modified red blood cell and molecules on the surface of a pathogen and/or cancerous cell. Aptamers specific for virtually any class of molecules may be isolated from a large library of 10¹⁴ to 10¹⁵ random oligonucleotide sequences using an iterative in vitro selection procedure often termed “systematic evolution of ligands by exponential enrichment” (SELEX; Cao et al., Current Proteomics 2:31-40 (2005); Proske et al., Appl. Microbiol. Biotechnol. 69:367-374 (2005), each of which is incorporated herein by reference).

Molecular beacons may be dual labeled aptamer probes with a donor fluorophore at one end and an acceptor fluorophore or quencher at the other end. Upon binding of a specific target, the aptamer is configured to undergo a conformational shift such that the distance between the donor fluorophore and the acceptor fluorophore or quencher is altered, leading to a change in detectable fluorescence. This phenomenon is referred to as fluorescence resonance energy transfer (FRET). FRET is a distance-dependent interaction between the electronic excited states of two dye molecules in which excitation is transferred from a donor molecule to an acceptor molecule without emission of a photon. In some instances, interaction of a donor molecule with an acceptor molecule may lead to a shift in the emission wavelength associated with excitation of the acceptor molecule. In other instances, interaction of a donor molecule with an acceptor molecule may lead to quenching of the donor emission. As such, an aptamer-based molecular beacon may be used to monitor changes in the fluorescent properties of the aptamer-based molecular beacon in response to binding a chemical entity such as, for example, a molecule on the surface of a target cell.

A variety of donor and acceptor fluorophore pairs may be considered for FRET associated with an aptamer-based molecular beacon including, but not limited to, fluorescein and tetramethylrhodamine; IAEDANS and fluorescein; fluorescein and fluorescein; and BODIPY FL and BODIPY FL. A number of Alexa Fluor (AF) fluorophores (from Molecular Probes-Invitrogen, Carlsbad, Calif., USA) may be paired with other AF fluorophores for use in FRET. Some examples include AF 350 with AF 488; AF 488 with AF 546, AF 555, AF 568, or AF 647; AF 546 with AF 568, AF 594, or AF 647; AF 555 with AF594 or AF647; AF 568 with AF6456; and AF594 with AF 647.

Red blood cells may be modified with a cell-surface receptor that signals either directly or indirectly in response to ligand binding. As an example, a G-protein-coupled receptor (GPCR) associated with a modified red blood cell may be used as an activatable molecular marker to monitor binding of a modified red blood cell to a target. The vast majority of GPCRs internalize from the cell surface into acidic endosomes in response to agonist challenge (Milligan, DDT 8:579-585 (2003), which is incorporated herein by reference). As such, a GPCR may be labeled with a pH sensitive dye which upon entering the acidic environment of the endosome changes its emission properties. The GPCR may be labeled with CypHer5™, for example, which is a red-excited, pH-sensitive cyanine dye that is non-fluorescent at pH 7.4 and maximally fluorescent at pH 5.5 and is ideally suited for monitoring internalization of GPCRs (available from Amersham Biosciences, Piscataway, N.J., USA). CypHer5™ may be attached to a protein by conjugation of CypHer5™ mono NHS ester to amine groups on the surface of the protein (See, e.g., Adie et al., Biotechniques 33:1152-1157 (2002), which is incorporated herein by reference). In the case of labeling a GPRC or other cell surface receptor, the receptor may be directly labeled with CypHer5™. Alternatively, a GPCR may be indirectly labeled by interaction with a CypHer5™ labeled antibody specific for that receptor (See, e.g., Adie et al., Biotechniques 33:1152-1157 (2002), which is incorporated herein by reference). CypHer5™ signaling is monitored at an emission wavelength of 695 nm using an excitation wavelength of 633 nm. Other pH sensitive dyes that might be used for labeling a GPCR or other cell surface receptor include but are not limited to fluoroscein isothiocyanate (FITC), 1,4(and 5)-benzenedicarboxylic acid, 2-[10-(dimethylamino)-4-fluoro-3-oxo-3H-benzo[c]xanthen-7-yl] (carboxy SNARF-4F), 2′,7′-Bis(2-carboxylethyl)-5(6)-carboxyfluorescein (BCECF).

### II. Methods of Treatment

In an aspect, one or more methods of treatment include providing one or more modified red blood cells to a subject; wherein the one or more modified red blood cells are associated with one or more target recognition moieties. In an aspect, one or more methods of treatment include providing one or more modified red blood cells to a subject; wherein the one or more modified red blood cells include one or more target recognition moieties. In an embodiment, the target recognition moieties are designed to recognize one or more neoplastic cells or pathogens. In an embodiment, activation of the target-binding agent and subsequent excitation of the photoactivatable molecule may cause release of a therapeutic agent (e.g., an antibiotic or chemotherapeutic agent) from the modified red blood cell. In other embodiments, the modified red blood cells include one or more fusion molecules that are designed to participate in a fusion of the modified red blood cells with the target cells.

Briefly, the modified red blood cells are administered to the subject before the target tissue, target composition or subject is subjected to electromagnetic radiation. The composition may be administered in a pharmaceutical formulation as described above. The dose of the modified red blood cells for an optimal therapeutic benefit can be determined clinically. A certain length of time is allowed to pass for the circulating or locally delivered modified red blood cells to be taken up by the target tissue. The unbound modified red blood cells are cleared from the circulation during this waiting period, or additional time can optionally be provided for clearing of the unbound modified red blood cells from non-target tissue. The waiting period will be determined clinically and may vary depending on the composition of the composition.

At the conclusion of this waiting period, a light source is used to excite the bound photoactivatable molecule. The light source may provide non-coherent (non-laser) or coherent (laser) light. For example, non-coherent light sources include, but are not limited to, mercury or xenon arc lamps with optical filters, tungsten lamps, cold cathode fluorescent lamps, halogen lamps, light emitting diodes (LEDs), LED arrays, incandescent sources, and other electroluminescent devices. Lamp sources are used when fine definition of the illumination region is not required, or when a large region is to be illuminated. Focused non-coherent light can be used to illuminate small regions, such as by using lenses to focus the light or optical fibers to direct or deliver the light. Laser sources are usually used to illuminate small, well-defined regions, because of their higher specific radiance and more readily controlled beam properties. Coherent light sources include, but are not limited to, dye lasers, argon ion lasers, laser diodes, tunable lasers, Ti-sapphire lasers, Ruby lasers, Alexandrite lasers, Helium-Neon lasers, GaAlAs and InGaAs diode lasers, Nd-YLF lasers, Nd-glass lasers, Nd-YAG lasers and fiber lasers. For example, lasers are often used as excitation sources in confocal equipment, and to create very high flux. Laser sources are limited in that they emit a restricted, often discrete set of wavelengths in contrast to lamps, which generally produce a continuous spectrum that can be filtered to provide any desired band within a certain range.

The area of illumination is determined by the location and dimension of the pathologic region to be detected, diagnosed or treated. The duration of illumination period will depend on whether detection or treatment is being performed, and can be determined empirically. A total or cumulative period of time anywhere from between about 1 min and 72 h can be used. In an embodiment, the illumination period is between about 4 min and 48 h. In another embodiment, the illumination period is between about 30 min and 24 h.

The total fluence (i.e., power) or energy of the light used for irradiating is from about 10 Joules and about 25,000 Joules; in an embodiment, the total fluence is from about 100 Joules and about 20,000 Joules or from about 500 Joules and about 10,000 Joules. Light of a wavelength and fluence sufficient to produce the desired effect is selected, whether for detection by fluorescence or for therapeutic treatment to destroy or impair a target tissue or target cell. Light having a wavelength corresponding at least in part with the characteristic light absorption wavelength of the photosensitizing agent is used for irradiating the target issue.

The power delivered by the light used is measured in watts, where 1 watt is equal to 1 joule/sec. Intensity is the power per area. Thus, intensity may be measured in watts/cm². Therefore, the intensity of the light used for irradiating may be between about 5 mW/cm² to about 500 mW/cm². Since the total fluence or amount of energy of the light in Joules is divided by the duration of total exposure time in seconds, the longer the amount of time the target is exposed to the irradiation, the greater the amount of total energy or fluence may be used without increasing the amount of the intensity of the light used. The methods typically employ an amount of total fluence of irradiation that is sufficiently high to excite the photoactivatable molecule of the target-binding agent.

In an embodiment of using the modified red blood cells disclosed herein for photodynamic therapy, the modified red blood cells are injected into the mammal, e.g., human, to be diagnosed or treated. The level of injection is usually between about 0.1 and about 0.5 μmol/kg of body weight. In the case of treatment, the area to be treated is exposed to light at the desired wavelength and energy, e.g., from about 10 to 200 J/cm². In the case of detection, fluorescence is determined upon exposure to light at a wavelength sufficient to cause the target-binding agent to fluoresce at a wavelength different than that used to illuminate the conjugate. The energy used in detection is sufficient to cause fluorescence and is usually significantly lower than is required for treatment.

The following sections will describe particular diseases or conditions that may be treated using the modified red blood cells.

A. Methods of Treating Cancer and Other Hyperproliferative Disorders

A neoplasm or tumor is an abnormal tissue growth resulting from neoplastic cells, i.e., cells that proliferate more rapidly and uncontrollably than normal cells. Usually partially or completely structurally disorganized, neoplasms lack functional coordination with the corresponding normal tissue. Neoplasms usually form a distinct tissue mass that may be either benign (tumor) or malignant (cancer). In addition to structural disorganization, cancer cells usually regress to more primitive or undifferentiated states (anaplasia), although morphologically and biochemically, they may still exhibit many functions of the corresponding wild-type cells. Carcinomas are cancers derived from epithelia; sarcomas are derived from connective tissues. In some cases, cancers may not be associated with a tumor, but like the affected tissue, is defuse, e.g., leukemias.

The modified red blood cells may be used to target neoplastic cells and designate those cells for damage or destruction. For example, the photoactivatable molecule of the modified red blood cells may act upon the neoplastic cells directly by bringing the cells in contact with singlet oxygen. Alternatively, the modified red blood cells may comprise red blood cells loaded with one or more therapeutic agents, e.g., a chemotherapeutic or antineoplastic agent, which is released from the modified red blood cell at the desired location. Consequently, the neoplastic cell is brought into close contact with a relatively high concentration of the therapeutic agent.

As described above, the modified red blood cells may be loaded with one or more chemotherapeutic agents for targeted delivery to a neoplastic cell. Examples of chemotherapeutic or antineoplastic agents include, but are not limited to, an alkylating agent; cisplatin; carboplatin; oxaliplatin; mechlorethamine; cyclophosphamide; chlorambucil; anti-metabolite compound; azathioprine; mercaptopurine; alkaloids; terpenoids; vinca alkaloid; vincristine; vinblastine; vinorelbine; vindesine; podophyllotoxin; taxanes; taxol; docetaxel; paclitaxel; topoisomerase inhibitors; camptothecins; irinotecan; topotecan; amsacrine; etoposide; etoposide phosphate; and teniposide; epipodophyllotoxins; antitumour antibiotics; dactinomycin; trastuzumab (Herceptin), cetuximab, and rituximab (Rituxan or Mabthera); Bevacizumab (Avastin); finasteride; tamoxifen; gonadotropin-releasing hormone agonists (GnRH); and goserelin.

In an embodiment, the at least one therapeutic agent is included in one or more of internal to the lipid surface, embedded in the lipid surface, or transversing the lipid surface. In an embodiment, the at least one therapeutic agent includes at least a portion of one of an organic or inorganic small molecule, proteinoid, nucleic acid, peptide, polypeptide, protein, glycopeptide, glycolipid, lipoprotein, lipopolysaccharide, sphingolipid, glycosphingolipid, glycoprotein, peptidoglycan, lipid, carbohydrate, metalloprotein, proteoglycan, vitamin, mineral, amino acid, polymer, copolymer, monomer, prepolymer, cell receptor, adhesion molecule, cytokine, chemokine, immunoglobulin, antibody, antigen, extracellular matrix constituent, cell ligand, oligonucleotide, element, hormone, transcription factor, or contrast agent.

In an embodiment, the polymer or co-polymer includes at least one of polyester, polylactic acid, polylactic-co-glycolic acid, cellulose, nitrocellulose, urea, urethane, or other polymer. In an embodiment, the at least one therapeutic agent includes at least one of calcium, carbon, nitrogen, sulfur, nitrate, nitrite, copper, magnesium, selenium, boron, sodium, aluminum, phosphorus, potassium, titanium, chromium, manganese, iron, nickel, zinc, silver, barium, lead, vanadium, tin, strontium, or molybdenum. In an embodiment, the at least one therapeutic agent includes at least one of insulin, clacitonin, lutenizing hormone, parathyroid hormone, somatostatin, thyroid stimulating hormone, vasoactive intestinal polypeptide, tumor necrosis factor, endostatin, angiostatin, anti-angiogenic antithrombin II, fibronectin, prolactin, thrombospondin I, laminin, procollagen, collagen, integrin, steroid, corticosteroid, allergen (for example, an agent that elicits a hyper-immune or hypersensitive response), self-antigen (for example, an antigen involved in autoimmune disease or disorder), virus antigen, microorganism antigen, T cell receptor ligand, T cell receptor, or lipase. In an embodiment, the virus antigen includes at least one antigen from one or more of a double-stranded DNA virus, single-stranded DNA virus, double-stranded RNA virus, (+) single-strand RNA virus, (−) single-strand RNA virus, single-strand RNA-Reverse Transcriptase virus, or double-stranded DNA-Reverse Transcriptase virus.

In an embodiment, the at least one therapeutic agent includes at least one vaccine. In an embodiment, the at least one vaccine includes at least one of an antigenic peptide, antigenic protein, or antigenic carbohydrate. In an embodiment, the at least one vaccine includes at least one of an envelope protein, capsid protein, surface protein, toxin, polysaccharide, or oligosaccharide. In an embodiment, the composition further includes at least one adjuvant.

In an embodiment, the at least one therapeutic agent includes at least one cytokine. In an embodiment, the at least one cytokine includes at one of Interleukin-1, Interleukin-2, Interleukin-3, Interleukin-4, Interleukin-5, Interleukin-6, Interleukin-7, Interleukin-8, Interleukin-9, Interleukin-10, Interleukin-11, Interleukin-12, Interleukin-13, Interleukin-14, Interleukin-15, Interleukin-16, Interleukin-17, Interleukin-18, Interleukin-19, Interleukin-20, Interleukin-21, Interleukin-22, Interleukin-23, Interleukin-24, Interleukin-25, Interleukin-26, Interleukin-27, Interleukin-28, Interleukin-29, Interleukin-30, Interleukin-31, Interleukin-32, Interleukin-33, Interleukin-34, Interleukin-35, Interleukin-36, Interleukin-37, Interleukin-38, Interleukin-39, Interleukin-40, Interleukin-41, Interleukin-42, Interferon-γ, Interferon-α, Interferon-β, Transforming Growth factor, Granulocyte Macrophage-Colony Stimulating Factor, Macrophage-Colony Stimulating Factor, Scarecrow, Erythropoietin, Granulocyte-Colony Stimulating Factor, Leukemia Inhibitory Factor, Oncostatin M, Ciliary Neurotrophic Factor, Growth Hormone, Prolactin, Fibroblast Growth factor, Nerve Growth factor, Platelet Derived Growth factor, Epidermal Growth factor, Fas, Fas ligand, CD40, CD27, CD4, CD8, CD2, CD3, Tumor Necrosis Factor-α, or Tumor Necrosis Factor-β.

In an embodiment, the at least one therapeutic agent includes at least one chemokine. In an embodiment, the at least one chemokine includes at least one of CXCR1, CXCR2, CXCR3, CXCR4, CXCR5, CCR1, CCR2, CCR3, CCR4, CCR5, CCR6, CCR7, CCR8, CCR9, IL-8, GROα, GROβ, GROγ, ENA-78, LDGF-PBP, GCP-2, PF4, Mig, IP-10, SDF-1α/β, BUNZO, STRC33, I-TAC, BLC, BCA-1, MIP-1α, MIP1-β, MDC, TECK, TARC, RANTES, HCC-1, HCC-4, DC-CK1, MIP-3α, MIP-3β, MCP-1, MCP-2, MCP-3, MCP-4, eotaxin, MPIF-2, I-309, MIP-5, HCC2, MPIF-1, 6CKine, CTACK, MEC, lymphotactin, fractalkine, CCL1, CCL2, CCL3, CCL4, CCL5, CCL6, CCL7, CCL8, CCL9/CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CCL29, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, CXCL18, CXCL19, CXCL20, CXCL21, CXCL22, XCL1, XCL2, XCL3, XCL4, XCL5, CX3CL1, CX3CL2, or CX3CL3.

In an embodiment, the at least one therapeutic agent includes at least one prodrug or precursor compound. In an embodiment, the at least one prodrug or precursor compound includes at least one glucuronide prodrug. In an embodiment, the at least one glucuronide prodrug includes at least one glucuronide of epirubicin, 5-fluorouracil, 4-hydroxycyclophosphamide, or 5-fluorocytosine. In an embodiment, the at least one prodrug or precursor compound includes 5-(aziridin-1-yl)-2,4-dinitrobenzamide. In an embodiment, the at least one therapeutic agent includes at least one converting enzyme active with the at least one prodrug or precursor compound. In an embodiment, the at least one enzyme includes at least one of β glucuronidase or cytosine deaminase. In an embodiment, the at least one enzyme includes nitroreductase or nitroreductase-like compound. In an embodiment, the at least one therapeutic agent includes at least a portion of an antibody expressed on the surface of the artificial antigen presenting cell.

B. Methods of Treating a Pathogen Infections

1. Bacterial Infections

Bacteremia is the presence of bacteria in the blood. Bacteremia has many possible causes, including dental procedures or even vigorous toothbrushing; catheterization of an infected lower urinary tract; surgical treatment of an abscess or infected wound; and colonization of indwelling devices, especially IV and intracardiac catheters, urethral catheters, and ostomy devices and tubes. Gram-negative bacteremia secondary to infection usually originates in the GU or GI tract, or the skin in patients with decubitus ulcers. Chronically ill and immunocompromised patients have an increased risk of gram-negative bacteremia. They may also develop bacteremia with gram-positive cocci, anaerobes, and fungi. Staphylococcal bacteremia is common in injection drug users. Bacteroides bacteremia may develop in patients with infections of the abdomen and the pelvis, particularly the female genital tract.

Metastatic infection of the meninges or serous cavities, such as the pericardium or larger joints, can result from transient or sustained bacteremia. Metastatic abscesses may occur almost anywhere. Multiple abscess formation is especially common with staphylococcal bacteremia. Bacteremia may cause endocarditis, most commonly if the pathogen is an Enterococcus, Streptococcus, or Staphylococcus, and less commonly with gram-negative bacteremia and fungemia. Patients with valvular heart disease, prosthetic heart valves, or other intravascular prostheses are predisposed to endocarditis, which may occur after certain dental procedures. Staphylococci can cause gram-positive bacterial endocarditis, particularly in injection drug users, and may involve the tricuspid valve. The bacteria most likely to cause bacteremia include members of the Staphylococcus, Streptococcus, Pseudomonas, Haemophilus, and Escherichia (E. coli) genera.

Bacterial diseases or disorders that can be treated or prevented by the use of the modified red blood cells include, but are not limited to, Mycobacteria, Rickettsia, Mycoplasma, Neisseria meningitides, Neisseria gonorrheoeae, Legionella, Vibrio cholerae, Streptococci, Staphylococcus aureus, Staphylococcus epidermidis, Pseudomonas aeruginosa, Corynobacteria diphtheriae, Clostridium spp., enterotoxigenic Escherichia coli, and Bacillus anthracis. Other pathogens for which bacteremia has been reported at some level include the following: Rickettsia, Bartonella henselae, Bartonella quintana; Coxiella burnetii; chlamydia; Mycobacterium leprae; Salmonella; shigella; Yersinia enterocolitica; Yersinia pseudotuberculosis; Legionella pneumophila; Mycobacterium tuberculosis; Listeria monocytogenes; Mycoplasma spp.; Pseudomonas fluorescens; Vibrio cholerae; Haemophilus influenzae; Bacillus anthracis; Treponema pallidum; Leptospira; Borrelia; Corynebacterium diphtheriae; Francisella; Brucella melitensis; Campylobacter jejuni; Enterobacter; Proteus mirabilis; Proteus; and Klebsiella pneumoniae.

A red blood cell may be modified with a moiety that allows the cell to target bacteria existing in the blood or at precise locations within the body. A target recognition moiety may be, for example, an antibody, antibody fragment, single chain antibody, DNA and/or RNA oligonucleotide, leptin, peptide, peptide nucleic acid (PNA), protein, receptor, drug, ligand, enzyme, and/or substrate, that is capable of specifically binding a target molecule associated with a bacteria.

In an embodiment, a red blood cell may be modified with a targeting antibody that specifically recognizes and targets the modified red blood cell to bacteria (See, e.g., U.S. Pat. No. 6,506,381 B1; U.S. Patent Application 2004/0033232 A1; U.S. Patent Application 2006/0018912 A1, each of which is incorporated herein by reference). The targeting antibody directed against a specific marker on the surface of the target cell may be generated using standard procedures. Alternatively, the targeting antibody may be commercially available.

One or more red blood cells may be modified with a target recognition moiety that is a cellular receptor that recognizes and/or binds to bacteria. For example, CD14, which is normally associated with monocyte/macrophages is known to bind lipopolysaccharide associated with gram negative bacteria as well as lipoteichoic acid associated with the gram positive bacteria Bacillus subtilis (See, e.g., Fan et al., Infect. Immun. 67:2964-2968 (1999), which is incorporated herein by reference). Other examples of cellular receptors include but are not limited to adenylate cyclase (Bordatella pertussis), Gal alpha 1-4Gal-containing isoreceptors (E. coli), glycoconjugate receptors (enteric bacteria), Lewis(b) blood group antigen receptor (Heliobacter pylori), CR3 receptor, protein kinase receptor, galactose N-acetylgalactosamine-inhibitable lectin receptor, and chemokine receptor (Legionella), annexin I (Leishmania mexicana), ActA protein (Listeria monocytogenes), meningococcal virulence associated Opa receptors (Meningococcus), \acute{α}5β3 integrin (Mycobacterium avium-M), heparin sulphate proteoglycan receptor, CD66 receptor, integrin receptor, membrane cofactor protein, CD46, GM1, GM2, GM3, and CD3 (Neisseria gonorrhoeae), KDEL receptor (Pseudomonas), epidermal growth factor receptor (Salmonella typhiurium), β1 integrin (Shigella), and nonglycosylated J774 receptor (Streptococci) (See, e.g., U.S. Patent Application 2004/0033584 A1, which is incorporated herein by reference).

A modified red blood cell may include an antibody or aptamer that binds a specific bacterium of interest. As such, the antibody may bring the red blood cell into close proximity to the bacteria. The red blood cell may be further modified with an additional component that has the ability to breach the outer membrane/cell wall of the bacterium such as, for example, lysozymes, bacteriocidal permeability increasing peptides, and other pore forming antimicrobials (See, e.g., U.S. Pat. No. 6,506,381 B1, which is incorporated herein by reference). For example, Zaitsev et al., (Blood 108:1895-1902 (2006), which is incorporated herein by reference) describe methods for modifying a red blood cell with a serine protease by linking the protease to an antibody to CR1, an abundant protein component of the red blood cell membrane. In this instance, the serine protease, tissue plasminogen activator (tPA), attached to the red blood cells retained its enzymatic activity in vivo. As such, lysozyme which hydrolyses 1,4-beta-linkages between N-acetylmuramic acid and N-acetyl-D-glucosamine residues in a peptidoglycan and between N-acetyl-D-glucosamine residues in chitodextrins of some bacteria may be similarly attached to the surface of a modified red blood cell through conjugation to a red blood cell binding antibody, for example. Alternatively, lysozyme may be expressed on the surface of a modified red blood cell as part of a membrane associated fusion protein, for example. Fusion proteins containing lysozyme have been described (See, e.g., U.S. Pat. Nos. 5,993,809 and 7,045,677, each of which is incorporated herein by reference). In addition, fusion proteins have been described that include a secreted protein that is retained in association with the exterior of a cell by fusion to a protein with a membrane anchor domain (See, e.g., U.S. Patent Application 2006/0068388 A1, which is incorporated herein by reference).

Alternatively, a modified red blood cell may include an antibody or aptamer that binds a specific bacterium of interest. In addition, the modified red blood cell may include one or more additional antibodies and/or aptamers to which is reversibly attached a therapeutic agent (See, e.g., U.S. Patent Application 2003/0215454 A1, which is incorporated herein by reference). In some instances, the red blood cell binds to its target and due to the concave nature of the red blood cell creates a small volume of space into which a subset of the therapeutic agent may diffuse to establish an equilibrium. As therapeutic agent is taken up by the target cell, more therapeutic agent is released from the modified red blood cell.

The modified red blood cell may be modified with an antibody and/or aptamer, for example, that specifically binds a target cell. Upon binding to the modified red blood cell, the target cell is immobilized and may be cleared in accordance with the body's red blood cell clearing mechanism through the phagocytic cells of the reticuloendothelial system.

In some instances, a therapeutic agent may be selectively released from a modified red blood cell using ultrasound energy. For example, red blood cells that have been sensitized with an electrical field are more sensitive to ultrasound-induced disruption of the cell membrane than normal, untreated red blood cells (See, e.g., U.S. Patent Application 2004/0071664, which is incorporated herein by reference). As such, modified red blood cells loaded with a therapeutic agent may be sensitized ex vivo with an electrical field prior to transfusion of the cells into an individual. The sensitizing electrical field may be as strong as that used for electroporation of a therapeutic agent into a modified red blood cell. Alternatively, lower electrical field strengths may be used. In general, electrical field strengths may range, for example, from about 0.1 kVolts/cm to about 10 kVolts/cm (See, e.g., U.S. Patent Applications 2002/0151004 A1 and 2004/0071664 A1, each of which is incorporated herein by reference). Ultrasound energy with a power density ranging from 0.05 to 100 W cm⁻² and frequency ranging from 0.015 to 10.0 MHz over a time frame ranging from 10 milliseconds to 60 minutes may be used to disrupt the sensitized red blood cells and induce release of the loaded therapeutic agent (See, e.g., U.S. Patent Application 2004/0071664, which is incorporated herein by reference). The sensitization step may be combined with the loading step using a specific device such as that described in U.S. Pat. No. 6,495,351 B2, which is incorporated herein by reference.

Examples of therapeutic agents (i.e., antibiotics) include, but are not limited to, beta-lactam compounds (penicillin, methicillin, nafcillin, oxacillin, cloxacillin, dicloxacilin, ampicillin, ticarcillin, amoxicillin, carbenicillin, piperacillin); cephalosporins & cephamycins (cefadroxil, cefazolin, cephalexin, cephalothin, cephapirin, cephradine, cefaclor, cefamandole, cefonicid, cefuroxime, cefprozil, loracarbef, ceforanide, cefoxitin, cefmetazole, cefotetan, cefoperazone, cefotaxime, ceftazidine, ceftizoxine, ceftriaxone, cefixime, cefpodoxime, proxetil, cefdinir, cefditoren, pivoxil, ceftibuten, moxalactam, cefepime); other beta-lactam drugs (aztreonam, clavulanic acid, sulbactam, tazobactam, ertapenem, imipenem, meropenem); cell wall membrane active agents (vancomycin, teicoplanin, daptomycin, fosfomycin, bacitracin, cycloserine); tetracyclines (tetracycline, chlortetracycline, oxytetracycline, demeclocycline, methacycline, doxycycline, minocycline, tigecycline); macrolides (erythromycin, clarithromycin, azithromycin, telithromycin); clindamycin; choramphenicol; quinupristin-dalfopristin; linezolid; aminoglycosides (streptomycin, neomycin, kanamycin, amikacin, gentamicin, tobramycin, sisomicin, netilmicin); spectinomycin; sulfonamides (sulfacytine, sulfisoxazole, silfamethizole, sulfadiazine, sulfamethoxazole, sulfapyridine, sulfadoxine); trimethoprim; pyrimethamine; trimethoprim-sulfamethoxazole; fluoroquinolones (ciprofloxacin, gatifloxacin, gemifloxacin, levofloxacin, lomefloxacin, moxifloxacin, norfloxacin, ofloxacin); colistimethate sodium, methenamine hippurate, methenamine mandelate, metronidazole, mupirocin, nitrofurantoin, and polymyxin B. Examples of anti-mycobacteria drugs include, but are not limited to: isoniazid, rifampin, rifabutin, rifapentine, pyrazinamide, ethambutol, ethionamide, capreomycin, clofazimine, and dapsone.

2. Methods of Treating Fungal Infection

Fungemia (also known as candidemia, candedemia, and invasive candidiasis) is the presence of fungi or yeasts in the blood. The most commonly known pathogen is Candida albicans, causing roughly 70% of fungemias, followed by Candida glabrata with 10%, and Aspergillus with 1%. However, the frequency of infection by T. glabrata, Candida tropicalis, C. krusei, and C. parapsilosis is increasing, especially when significant use of fluconazole is common.

A red blood cell may be modified with a moiety that allows the cell to target fungal cells in the subject's body. A target recognition moiety may be, for example, an antibody, antibody fragment, single chain antibody, DNA and/or RNA oligonucleotide, leptin, peptide, peptide nucleic acid (PNA), protein, receptor, drug, ligand, enzyme, and/or substrate, that is capable of specifically binding a target molecule associated with a fungal cell.

In an embodiment, a red blood cell may be modified with a targeting antibody that specifically recognizes and targets the modified red blood cell to fungi. The targeting antibody directed against a specific marker on the surface of the target cell may be generated using standard procedures. Alternatively, the targeting antibody may be commercially available.

In an embodiment, a modified red blood cell may be loaded with an antifungal agent that is released upon contact with the fungal cell. Examples of antifungal agents includes, but is not limited to: allylamines; terbinafine; antimetabolites; flucytosine; azoles; fluconazole; itraconazole; ketoconazole; ravuconazole; posaconazole; voriconazole; glucan synthesis inhibitors; caspofungin; micafungin; anidulafungin; polyenes; amphotericin B; amphotericin B Lipid Complex (ABLC); amphotericin B Colloidal Dispersion (ABCD); liposomal amphotericin B (L-AMB); liposomal nystatin; and griseofulvin.

3. Methods of Treating a Parasitic Infection

In an embodiment, the modified red blood cell may be administered to a subject for the treatment of a parasitic infection. The targeting compositions may be directed to intestinal or blood-borne parasites, including protozoa. Typically, blood-borne parasites are transmitted through an arthropod vector. Most important arthropod for transmitting parasitic infections are mosquitoes. Mosquitoes carry malaria and filarial nematodes. Biting flies transmit African trypanosomiasis, leishmaniasis and several kinds of filariasis. Examples of parasites include, but are not limited to, trypanosomes; haemoprotozoa and parasites capable of causing malaria; enteric and systemic cestodes including taeniid cestodes; enteric coccidians; enteric flagellate protozoa; filarial nematodes; gastrointestinal and systemic nematodes and hookworms.

A red blood cell may be modified with a moiety that allows the cell to target the parasite or particular cells of the parasite. A target recognition moiety may be, for example, an antibody, antibody fragment, single chain antibody, DNA and/or RNA oligonucleotide, leptin, peptide, peptide nucleic acid (PNA), protein, receptor, drug, ligand, enzyme, and/or substrate, that is capable of binding a target molecule associated with the parasite.

In an embodiment, a red blood cell may be modified with a targeting antibody that recognizes and targets the red blood cell to the parasite. The targeting antibody directed against a marker on the surface of the target may be generated using standard procedures. Alternatively, the targeting antibody may be commercially available. In an embodiment, a modified red blood cell may be loaded with an anti-parasitic agent that is released upon contact with the parasite. Examples of anti-parasitic drugs include, but are not limited to: antiprotozoal agents; eflornithine; furazolidone; melarsoprol; metronidazole; ornidazole; paromomycin sulfate; pentamidine; pyrimethamine; tinidazole; antimalarial agents; quinine; chloroquine; amodiaquine; pyrimethamine; sulphadoxine; proguanil; mefloquine; halofantrine; primaquine; artemesinin and derivatives thereof; doxycycline; clindamycin; benznidazole; nifurtimox; antihelminthics; albendazole; diethylcarbamazine; mebendazole; niclosamide; ivermectin; suramin; thiabendazole; pyrantel pamoate; levamisole; piperazine family; praziquantel; triclabendazole; octadepsipeptides; and emodepside.

4. Methods of Treating a Viral Infection

In an embodiment, the modified red blood may be administered to a subject for the treatment of a viral infection. A red blood cell may be modified with a moiety that allows the cell to target the virus or host cells of the virus. A target recognition moiety may be, for example, an antibody, antibody fragment, single chain antibody, DNA and/or RNA oligonucleotide, leptin, peptide, peptide nucleic acid (PNA), protein, receptor, drug, ligand, enzyme, and/or substrate, that is capable of specifically binding a target molecule associated with the virus.

In an embodiment, a red blood cell may be modified with a targeting antibody that specifically recognizes and targets the red blood cell to the virus. The targeting antibody directed against a specific marker on the surface of the virus may be generated using standard procedures. Alternatively, the targeting antibody may be commercially available. For example, the target recognition moieties of the modified red blood cells may be directed to clinically important viruses, including but not limited to adenovirus, coxsackievirus, hepatitis a virus, poliovirus, epstein-barr virus, herpes simplex, type 1, herpes simplex, type 2, human cytomegalovirus, human herpesvirus, type 8, varicella-zoster virus, hepatitis B virus, hepatitis C viruses, human immunodeficiency virus (HIV), influenza virus, measles virus, mumps virus, parainfluenza virus, respiratory syncytial virus, papillomavirus, rabies virus, and Rubella virus.

In an embodiment, a modified red blood cell may be loaded with an antiviral agent that is released upon contact with the virus. Examples of antiviral agents include: thiosemicarbazones; metisazone; nucleosides and nucleotides; acyclovir; idoxuridine; vidarabine; ribavirin; ganciclovir; famciclovir; valaciclovir; cidofovir; penciclovir; valganciclovir; brivudine; ribavirin, cyclic amines; rimantadine; tromantadine; phosphonic acid derivatives; foscarnet; fosfonet; protease inhibitors; saquinavir; indinavir; ritonavir; nelfinavir; amprenavir; lopinavir; fosamprenavir; atazanavir; tipranavir; nucleoside and nucleotide reverse transcriptase inhibitors; zidovudine; didanosine; zalcitabine; stavudine; lamivudine; abacavir; tenofovir disoproxil; adefovir dipivoxil; emtricitabine; entecavir; non-nucleoside reverse transcriptase inhibitors; nevirapine; delavirdine; efavirenz; neuraminidase inhibitors; zanamivir; oseltamivir; moroxydine; inosine pranobex; pleconaril; and enfuvirtide.

### III. Diagnostic and Imaging Methods

In one aspect, the disclosure provides methods of using modified red blood cells to deliver an imaging agent to a cell or a tissue within a subject. In an embodiment, the modified red blood cells may be engineered to express or carry one or more molecular markers; wherein the one or more molecular markers are configured to be activated by interaction with one or more molecules to be detected. For example, an aptamer-based molecular beacon may be located in the cytoplasm of a red blood cell and detect changes in cellular signaling associated with interaction of a modified red blood cell with a target.

In another embodiment, the red blood cells are loaded with an imaging agent that emits a detectable signal, such as light or other electromagnetic radiation. In another embodiment, the imaging agent is a radio-isotope, for example ³²P or ³⁵S or ⁹⁹Tc, or a molecule such as a nucleic acid, polypeptide, or other molecule, conjugated with such a radio-isotope. In an embodiment, the imaging agent is opaque to radiation, such as X-ray radiation. For example, the agent may comprise a radiolabelled antibody which specifically binds to defined molecule(s), tissue(s) or cell(s) in an organism.

In another embodiment, the imaging agent is a contrast dye. For example, an MRI contrast agent can comprise a paramagnetic contrast agent (such as a gadolinium compound), a superparamagnetic contrast agent (such as iron oxide nanoparticles), a diamagnetic agent (such as barium sulfate), and combinations thereof. Metal ions preferred for MRI include those with atomic numbers 21-29, 39-47, or 57-83, and, more typically, a paramagnetic form of a metal ion with atomic numbers 21-29, 42, 44, or 57-83. Particularly preferred paramagnetic metal ions are selected from the group consisting of Gd(III), Fe(III), Mn(II and III), Cr(III), Cu(II), Dy(III), Tb(III and IV), Ho(III), Er(III), Pr(III) and Eu(II and III). Gd(III) is particularly useful. Note that as used herein, the term “Gd” is meant to convey the ionic form of the metal gadolinium; such an ionic form can be written as GD(III), GD3+, etc. with no difference in ionic form contemplated. A CT contrast agent can comprise iodine (ionic or non-ionic formulations), barium, barium sulfate, Gastrografin (a diatrizoate meglumine and diatrizoate sodium solution), and combinations thereof. In another embodiment, a PET or SPECT contrast agent can comprise a metal chelate. Following administration of the contrast dye, the subject can be imaged using X-ray, MRI, CT, or PET scanning.

The compositions and methods described herein are further illustrated by the following examples, which should not be construed as limiting in any way.

### PROPHETIC EXAMPLES

### Prophetic Example 1

### Treatment of a Hyperproliferative Disorder

In this example, the modified red blood cells are used to treat a hyperproliferative disorder (e.g., cancer). In one instance, the modified red blood cells are targeted to surface antigens on a neoplastic cell, known or suspected to be present in a subject's body. Upon excitation with light of the appropriate wavelength and power, singlet oxygen radicals are generated, resulting in damage or destruction of the neoplastic cell. In another instance, the modified red blood cells are used to deliver a therapeutic agent to the neoplastic cell(s).

A photoactivatable molecule, such as a porphyrin, is conjugated, via an amide linkage, to a monoclonal antibody known to exhibit selective binding to an antigen expressed on the surface of a neoplastic cell. The antibody is also conjugated to a quenching agent such as a Dabcyl (4-(4′-dimethylaminophenylazo)benzoyl) group, by reaction with a commercially available agent such as dabcyl chloride. This target-binding agent is further modified by the addition of a suitable metal ion to an aqueous solution of the composition. The metal binds to the coordination pocket of the porphyrin ring-system and also coordinates the amine or azo group of the quenching group, ensuring that the quenching agent remains sufficiently close to the photoactivatable molecule to allow energy transfer and thereby quench the generation of singlet oxygen.

Next, red blood cells are isolated from the subject in need of treatment. In cases where it is desirable to deliver a therapeutic agent to the neoplastic cells, the cells are loaded with a chemotherapeutic agent, such as 5-fluorouracil, and biotinylated. The antibody is also conjugated with biotin and then linked to the biotinylated red blood cells by a streptavidin bridge to form an assembled target-binding agent. The assembled target-binding agent is mixed with a suitable excipient for intravenous administration to the subject. A therapeutically effective amount of this target-binding agent is administered to the subject.

Binding of the antibody to its target then disrupts the coordination binding environment, releasing the quencher molecule from the metal and allowing the quencher molecule to move away from the photoactivatable molecule, thereby activating the target-binding agent. After a sufficient time for the target-binding agent to bind to the intended target and clear from normal tissue, a light source of the appropriate wavelength is used to deliver a therapeutically useful amount of light to an area that includes the lesion or region of hyperproliferative tissue. The light causes the excitation of the photoactivable moiety, resulting in the production of a singlet oxygen radical molecule. The singlet oxygen radical molecule may act directly on the neoplastic cell, thereby damaging or destroying the cell. Alternatively, the singlet oxygen radical disrupts the cell membrane of the red blood cell, thereby releasing the chemotherapeutic agent. The chemotherapeutic agent is contacted with the neoplastic cell causing cell death.

The efficacy of treatment is assessed by reduction in the number of neoplastic cells or absence of the neoplastic cells; reduction in the tumor size; inhibition (i.e., slow to some extent and preferably stop) of tumor metastasis; inhibition, to some extent, of tumor growth; increase in length of remission, and/or relief to some extent, one or more of the symptoms associated with the specific cancer.

### Prophetic Example 2

### Treatment of a Pathogen Infection

In this example, the modified red blood cells are used to treat a pathogen infection (e.g, bacterial, fungal, viral or parasitic). In one instance, the modified red blood cells are targeted to surface antigens of the pathogen, where upon excitation with light of the appropriate wavelength and power, singlet oxygen radicals are generated, resulting in damage or destruction of the pathogen. In another instance, the modified red blood cells are used to deliver a therapeutic agent to the pathogen, known or suspected to have infected a subject.

A photoactivatable molecule, such as a porphyrin, is conjugated, via an amide linkage, to a monoclonal antibody known to exhibit selective binding to an antigen expressed on the surface of a pathogen, e.g., the bacterium Staphylococcus aureus. The antibody is also conjugated to a quenching agent such as a Dabcyl (4-(4′-dimethylaminophenylazo)benzoyl) group, by reaction with a commercially available agent such as dabcyl chloride. This target-binding agent is further modified by the addition of a suitable metal ion to an aqueous solution of the composition. The metal binds to the coordination pocket of the porphyrin ring-system and also coordinates the amine or azo group of the quenching group, ensuring that the quenching agent remains sufficiently close to the photoactivatable molecule to allow energy transfer and thereby quench the generation of singlet oxygen.

Next, red blood cells are isolated from the subject in need of treatment. In cases where it is desirable to use a therapeutic agent, the cells are loaded with the therapeutic agent (e.g., an antibiotic, antifungal, antiparasitic, or antiviral), such as ciprofloxacin, and biotinylated. The antibody is also conjugated with biotin and then linked to the biotinylated red blood cells by a streptavidin bridge to form an assembled target-binding agent. The assembled target-binding agent is mixed with a suitable excipient for intravenous administration to the subject. A therapeutically effective amount of this target-binding agent is administered to the subject.

Binding of the antibody to its pathogen target then disrupts the coordination binding environment, releasing the quencher molecule from the metal and allowing the quencher molecule to move away from the photoactivatable molecule, thereby activating the target-binding agent. After a sufficient time for the target-binding agent to bind to the intended target and clear from normal tissue, a light source of the appropriate wavelength is used to deliver a therapeutically useful amount to light to an area that includes the lesion. The light causes the excitation of the photoactivable moiety, resulting in the production of a singlet oxygen radical molecule. The singlet oxygen radical molecule may act on pathogen directly, thereby damaging or destroying the pathogen. Alternatively, the singlet oxygen radical disrupts the cell membrane of the red blood cell, which has been loaded with the therapeutic agent, thereby releasing the therapeutic agent. The therapeutic agent is contacted with the pathogen causing damage, death or inactivation of the pathogen.

The efficacy of treatment is assessed by reduction in the number of pathogen cells or absence of the pathogen cells; or reduction one or more of the symptoms associated with the infection.

### Prophetic Example 3

### Imaging a Target Tissue of a Subject

In this example, the modified red blood cells are used to transport an imaging agent, i.e. fluorescent molecule or radiocontrast dye, to a particular tissue or cell-type. A photoactivatable molecule, such as a porphyrin, is conjugated, via an amide linkage, to a monoclonal antibody known to exhibit selective binding to an antigen expressed in a particular tissue of the subject. The antibody is also conjugated to a quenching agent such as a Dabcyl (4-(4′-dimethylaminophenylazo)benzoyl) group, by reaction with a commercially available agent such as dabcyl chloride. This target-binding agent is further modified by the addition of a suitable metal ion to an aqueous solution of the composition. The metal binds to the coordination pocket of the porphyrin ring-system and also coordinates the amine or azo group of the quenching group, ensuring that the quenching agent remains sufficiently close to the photoactivatable molecule to allow energy transfer and thereby quench the generation of singlet oxygen.

Next, red blood cells are isolated from the subject in need of imaging. The cells are loaded with the imaging agent and biotinylated. The antibody is also conjugated with biotin and then linked to the biotinylated red blood cells by a streptavidin bridge to form an assembled modified red blood cell. The assembled modified red blood cell is mixed with a suitable excipient for intravenous administration to the subject. A therapeutically effective amount of this modified red blood cell is administered to the subject.

Binding of the antibody to its bacterial target then disrupts the coordination binding environment, releasing the quencher molecule from the metal and allowing the quencher molecule to move away from the photoactivatable molecule, thereby activating the target-binding agent. After a sufficient time for the target-binding agent to bind to the intended target and clear from normal tissue, a light source of the appropriate wavelength is used to deliver a useful amount to light to an area that includes the lesion. The light causes the excitation of the photoactivable moiety, resulting in the production of a singlet oxygen radical molecule, which disrupts the cell membrane of the red blood cell, thereby releasing the imaging agent, e.g., radiocontrast dye. The imaging agent is detected using X-ray, CT, or other means.

### Prophetic Example 4

### Construction of Artificial Antigen Presenting Cell with MHC Class II Receptor

An artificial antigen presenting cell (aAPC) is constructed from a MHC class II (MHCII) protein joined with an antigenic peptide (e.g., epitope) and a costimulatory molecule, B7.1. The MHCII-epitope-B7.1 complex is inserted in the lipid bilayer of a liposome.

An aAPC is constructed from a liposome including a lipid bilayer with an embedded MHCII protein, HLA-DR1, that includes a peptide epitope, influenza nucleoprotein amino acids 404-415 (NP 404-415). Methods of making single chain MHC II proteins joined to antigenic peptides are known in the art (see e.g., U.S. Pat. No. 7,141,656, which is incorporated herein by reference). Complementary DNA (cDNA) for the HLA-DR1 α chain and HLA-DR1 β chain are each obtained by molecular cloning using messenger RNA (mRNA) isolated from BLCL-K68 cells (a HLA-DR1 homozygous cell line). Methods to isolate mRNA, clone cDNA, and determine DNA sequences are known (see e.g., U.S. Pat. No. 7,141,656, ibid. and Sambrook and Russell, “Molecular Cloning: A Laboratory Manual”, (Third Edition, 2001, Cold Spring Harbor Laboratory Press, Woodbury, N.Y.), each of which is incorporated herein by reference). An oligonucleotide (available from Sigma-Aldrich Chem. Co., St. Louis, Mo.) encoding the influenza peptide, NP (404-415) is joined with the 5′ end of a cDNA segment encoding the HLA-DR1 β chain (see e.g., U.S. Pat. No. 7,141,656, Ibid). The NP (404-415)-DR1 β chain gene and a gene for DR1 α chain are inserted in a bicistronic mammalian cell expression vector (see e.g., Product Information Sheet: “pIRES Vector” available from Clontech Laboratories, Inc., Mountain View, Calif., which is incorporated herein by reference). A second mammalian cell expression vector encoding the costimulatory molecule B7.1 (also known as CD80) is constructed with an alternate selectable marker, dihydrofolate reductase (DHFR), to allow co-selection of the B7.1 vector and the HLA-DR1 vector with methotrexate and G418, respectively. Methods for molecular cloning and co-expression of MHC II and co-stimulatory genes are known in the art (see e.g., U.S. Pat. No. 7,439,335, which is incorporated herein by reference). Chinese hamster ovary (CHO) cells are co-transfected with HLA-DR1 and B7.1 vectors using Lipofectamine™ (available from Life Technologies Corp., Carlsbad, Calif.), and stable clones are selected for resistance to both G418 and methotrexate. To test for the expression of both proteins, the cells are stained with fluorescent antibodies and analyzed on a flow cytometer (antibodies, reagents, protocols and flow cytometers are all available from BD Biosciences, San Jose, Calif.). Stable CHO cell lines expressing both B7.1 and HLA-DR1 (with the joined epitope, NP 404-415) are expanded in a bioreactor to provide a source of HLA-DR1 and B7.1. Methods to purify proteins using, for example, affinity columns, is known in the art. See, e.g., J. Clin. Invest. 112 (6): 831-842 (2003); and Proc. Nat'l. Acad. Sci. USA 1998 95:11828-11833, each of which is incorporated herein by reference. Proper protein identified is made by immunoblotting with antibodies specific for HLA-DR1 and B7.1. In certain aspects, lipid rafts may alternatively be used for isolation and purification of proteins.

HLA-DR1 and B7.1 proteins are joined with a bispecific antibody (Bisp Ab) that recognizes both antigens. Methods to make Bisp Abs are known in the art (see e.g., Herrmann et al., Cancer Res. 68: 1221-1227, 2008 which is incorporated herein by reference). A bispecific single chain antibody with multiple variable region domains is derived from an anti-HLA-DR1 antibody (scFv) and an anti-B7.1 antibody (scFv). Methods to select human antibodies from phage display single chain variable fragment (scFv) libraries are known in the art (see e.g., Pansri et al., BMC Biotechnology 9(6): 2009; doi: 10.1186/1472-6750-9-6 which is incorporated herein by reference).

A DNA construct encoding a single chain Bisp Ab construct with tandem scFv regions is then inserted into a mammalian cell expression vector and transferred into SP2/0, a mouse myeloma cell line (available from American Type Culture Collection, Manassas, Va.). Production and purification of the Bisp Ab are well known in the art (see e.g., Herrman, Ibid.) Purified HLA-DR1 and B7.1 proteins are joined by a Bisp Ab that binds both proteins, and the joined proteins are incorporated in liposomes to create artificial antigen presenting cells.

Liposomes are prepared from cholesterol and L-α-phosphatidylcholine using methods known in the art (see e.g., U.S. Patent Application No. 2005/0208120, which is incorporated herein by reference). Cholesterol and L-α-phosphatidylcholine are combined at a molar ratio of 2:7 in chloroform. The chloroform is evaporated away using an argon stream. Next, the liposomes are resuspended in a 140 mM NaCl, 10 mM Tris HCl, 0.5% deoxycholate at pH 8, and sonicated for three minutes. The complexed HLA-DR1 and B7.1 joined by a Bisp Ab are inserted in the liposomes by combining the complexes with liposomes at a 1:10 molar ratio and dialyzing for 72 hours at 4° C. versus phosphate buffered saline. The liposomes are characterized to assess liposome size and the amount of HLA-DR1 and B7.1 protein incorporated in the liposomes. Liposome size is determined using dynamic light scattering and flow cytometry (see e.g., U.S. Patent Application No. 2005/0208120, Ibid). For example, liposomes containing HLA-DR may have a mean diameter of approximately 50 nanometers. To quantitate HLA-DR1 and B7.1 protein on the liposomes, the liposomes are analyzed on a flow cytometer after staining with FITC labeled anti-DR antibody. Liposomes are sorted based on FITC fluorescence, forward scatter and side scatter to isolate and count liposomes with HLA-DR1. HLA-DR and B7.1 protein on the liposomes is quantitated using an enzyme-linked immunosorbent assay (ELISA). Methods to analyze liposomes by flow cytometry and to quantitate HLA-DR and other proteins by ELISA are known in the art (see e.g., U.S. Patent Application No. 2005/0208120, Ibid.).

Artificial antigen presenting cells containing HLA-DR1 with the influenza epitope NP (404-415) fused to the DR1 β chain and B7.1 joined by a Bisp Ab can be purified prior to their use. The Bisp Ab, bound to aAPC via HLA-DR1 and B7.1, contains human kappa variable region (Vₖ) sequences which are recognized by an immunoaffinity ligand, protein L. An immunoaffinity column comprised of Protein L Agarose is used to purify the aAPC by binding the Bisp Ab bound to the aAPC. Methods to purify antibodies containing Vₖ regions are known in the art (see e.g., Instructions: Pierce® Protein L Agarose available from Pierce Biotechnology, Rockford, Ill. which is incorporated herein by reference). Bisp Ab containing human Vk regions are bound to a Protein L agarose column in Binding Buffer which contains 0.1 M phosphate and 0.15 M sodium chloride at pH 7.2. The column is washed with 2.5 to 4 column volumes of binding buffer and then the Bisp Ab-aAPC are eluted from the column with 2.5 column volumes of Elution Buffer which contains 0.10 M glycine, pH 2-3. At low pH Protein L releases Bisp Ab and allows the aAPC to flow through the column and be collected in a neutralizing buffer (e.g., 1M TrisHCl, pH 7.5).

Artificial APC containing HLA-DR1 with epitope NP (404-415) are used to stimulate human CD4+ T cells in vitro, ex vivo, and in vivo to promote anti-influenza immune responses. For example, CD4+ T lymphocytes are isolated from the peripheral blood an individual previously immunized with a standard influenza vaccine and incubated with aAPC bearing HLA-DR1-NP (404-415) and B7.1 in vitro for 72 hours at 37° C. in tissue culture media in 5% CO₂ in air. Methods for isolation, culture and evaluation of CD4+ T cells are known in the art (see e.g., U.S. Patent App. Publ. No. 2005/0208120, Ibid.). The CD4+ T cells are evaluated using flow cytometry to detect CD69, an activation antigen, on their cell surface and by measuring the amount of interleukin-2 (IL-2) produced by the CD4+ cells. Activated anti-influenza CD4+ T cells may be infused into the blood cell donor or into other HLA-matched individuals to promote anti-influenza immunity.

### Prophetic Example 5

### Construction of Artificial Antigen Presenting Cell with MHC-Peptide-B7.1 Fusion Protein with Proteinase-Activated Receptor (PAR2)

An artificial antigen presenting cell (aAPC) is constructed by expressing a fusion protein that contains a MHC class II (MHCII) protein, an antigenic peptide (i.e., an epitope), a co-stimulatory molecule, B7.1, and peptide sequences from the proteinase activated receptor 2 (PAR2). The fusion protein is expressed in red blood cell progenitor cells, and these are expanded and differentiated in vitro to yield red blood cells presenting joined MHC Class II and B7.1.

Artificial APC are constructed by transduction of red blood cell progenitor cells with lentiviral vectors encoding a fusion protein. The fusion protein encodes the influenza nucleoprotein epitope NP (404-415), the HLA-DR1 protein B7.1, and sequences from PAR2, including a cytoplasmic loop, transmembrane domain (TMD), and an exodomain with a protease cleavage site. Methods of making single chain MHC II proteins joined to antigenic peptides are known in the art (see e.g., Zhu et al., Eur. J. Immunol. 27: 1933-1941, 1997; and U.S. Pat. No. 7,141,656, each of which is incorporated herein by reference). Complementary DNA (cDNA) for the HLA-DR1 α chain and HLA-DR1 β chain are obtained by molecular cloning using messenger RNA (mRNA) from human lymphoblastoid cells (a HLA-DR1 homozygous cell line). Methods to isolate mRNA, clone cDNA and determine DNA sequences are known in the art (see e.g., U.S. Pat. No. 7,141,656, Ibid.; and Sambrook and Russell, “Molecular Cloning: A Laboratory Manual”, (Third Edition, 2001, Cold Spring Harbor Laboratory Press, Woodbury, N.Y.), each of which is incorporated herein by reference). An oligonucleotide (available from Sigma-Aldrich Chem. Co., St. Louis, Mo.) encoding the influenza nucleoprotein peptide NP (404-415) is joined with the 5′ end of a cDNA segment encoding the HLA-DR1 β chain (see e.g., U.S. Pat. No. 7,141,656, Ibid.). A construct encoding a single chain HLA-DR1 (scDR1) is constructed with the NP (404-415) epitope-DR1β chain (extracellular domain) and DR1α chain joined (see e.g., Zhu et al., Ibid.). The scDR1 construct is joined to DNA sequences derived from PAR2 and B7.1. The cytoplasmic domain of the scDR1 (derived from the carboxy terminus of the HLA-DR1 α chain) is joined to the first cytoplasmic loop (amino acids (a.a.) 101-107), the second TMD (a.a. 108-128), and part of the exodomain (a.a. 28-40) of PAR2, including a trypsin cleavage site (see Figures A and B). Methods and compositions to make PAR2 fusion proteins are known in the art (see e.g., Nystedt et al., Eur. J. Biochem. 232: 84-89, 1995 and Bae et al., J. Thromb. Haemost. 6: 954-961, 2008, which are incorporated herein by reference). The mature amino terminus of the B7.1 sequence is joined adjacent to the PAR2 trypsin cleavage site. A diagram of the encoded fusion protein (Figure A) displays the fusion protein starting at the amino terminus and encompassing NP (404-415), scDR1, PAR2 and B7.1 at the carboxyl terminus.

The DNA construct encoding the scDR1-B7.1 fusion protein is inserted into a lentivirus vector and expressed in red blood cell progenitor cells. Lentiviral vectors and methods of using the same for gene expression are known in the art (see e.g., “Lenti-X™ Lentiviral Expression Systems User Manual” available from Clontech Laboratories, Inc., Mountain View, Calif., which is incorporated herein by reference). DNA sequences encoding the scDR1-B7.1 fusion protein are cloned into a plasmid-based expression vector containing required elements for packaging the expression construct into virions. The plasmid is combined with a packaging mixture and transfected into a 293T cell line (available from American Type Culture Collection, Manassas, Va.) to produce a recombinant, non-replicating lentivirus. Lentiviral stocks with a titer of approximately 10⁵ to 10⁷ transducing units/ml are sufficient to transfer 10⁶-10⁸ hematopoietic stem cells (HSC) at a multiplicity of infection of 1.0. To determine the titer of the lentivirus stock, serial ten-fold dilutions of the stock are applied to a HT-1080 cell line (available from American Type Culture Collection, Manassas, Va.) and the number of transduced cells is counted after growth in puromycin since a puromycin resistance gene is incorporated in the lentiviral expression vector to allow selection of stably transduced cells. HSC are transduced with the lentiviral vector encoding the DR1-B7.1 fusion protein to create HSC that present joined NP (404-415)-scDRI and B7.1 on their plasma membrane. See Figure B.

Methods to obtain HSC from peripheral blood are known in the art (see e.g., Lane et al., Blood 85: 275-282, 1995, which is incorporated herein by reference). To mobilize HSC the donor is given granulocyte colony-stimulating factor (G-CSF; also known as filgrastim from Amgen Inc., Thousand Oaks, Calif.) 10 μg/kg/day subcutaneously for 4 days. HSC are harvested by leukapheresis on day 5 (see e.g., Lane et al., Ibid.). HSC are selected using magnetic beads and anti-CD34 antibodies (Magnetic beads, antibodies and protocols are available from Miltenyi Biotec, Inc., Auburn, Calif.). Approximately 10⁸ mononuclear CD34⁺ cells are obtained, and these are transduced with the lentiviral expression vector encoding the DR1-B7.1 fusion protein.

Hematopoietic stem cells transduced with the lentivirus encoding joined NP epitope, HLA-DR1 and B7.1 are treated with trypsin in vitro to cleave the trypsin site adjacent to B7.1 in the fusion protein. The trypsin cleavage site in PAR2 is cleaved by approximately 1 nM trypsin in vitro (see e.g., Nystedt et al., Ibid.), and since the normal range for human serum trypsin concentration is between 5.7-16.4 nM (Artigas et al., Postgrad. Med. J. 57: 219-222, 1981, which is incorporated herein by reference), it will also be cleaved in vivo. After trypsin cleavage of the DR1-B7.1 fusion protein, the HSC are tested for the expression of HLA-DR1 and B7.1. The cells are stained with fluorescent antibodies and analyzed on a flow cytometer (antibodies, reagents, protocols and flow cytometers are all available from BD Biosciences, San Jose, Calif.). HSC displaying DR1 and B7.1 on their cell surface are sorted based on immunofluorescence from fluor-conjugated antibodies. For example, anti-DR1 conjugated with fluorescein isothiocyanate (FITC) and anti-B7.1 conjugated with phycoerythrin (PE) is used to stain the HSC. Cells displaying both green and red fluorescence are sorted into a collection vessel. Conjugated antibodies, protocols and a FACS Vantage™ cell sorter are all available from BD Biosciences-Immunocytometry Systems, San Jose, Calif.

Isolated, double positive (DR1 and B7.1) HSC are cultured, expanded and differentiated ex vivo. For example, HSC isolated from peripheral blood are expanded and differentiated ex vivo into mature erythrocytes (Giarratana et al., Nature Biotech. 23: 69-74 2004; and U.S. Patent App. Pub. No. 2007/0218552; each of which is incorporated herein by reference. HSC expressing DR1 and B7.1 are subsequently cultured in modified serum-free medium supplemented with 1% bovine serum albumin (BSA), 120 μg/ml iron-saturated human 296ransferring, 900 ng/ml ferrous sulfate, 90 ng/ml ferric nitrate and 10 μg/ml insulin and maintained at 37° C. in 5% carbon dioxide in air.

Expansion and differentiation of the cell culture occurs in multiple steps. For example, in the initial growth step following isolation, the cells are expanded in the medium described above in the presence of multiple growth factors including, for example, hydrocortisone, stem cell factor, IL-3, and erythropoietin. In the second stage, the cells are co-cultured, for example, on an adherent stromal layer in the presence of erythropoietin. In a third stage, the cells are cultured on an adherent stromal layer in culture medium in the absence of exogenous factors. The adherent stromal layer includes murine MS-5 stromal cells, (see for example Issaad et al., Blood 81: 2916-2924, 1993 which is incorporated herein by reference. Alternatively, the adherent stromal layer includes mesenchymal stromal cells derived from adult bone marrow. The adherent stromal cells are maintained in RPMI supplemented with 10% fetal calf serum.

Various assays are performed to confirm the ex vivo differentiation of cultured hematopoietic stem cells into reticulocytes and erythrocytes, including, for example, microscopy, hematology, flow cytometry, deformability measurements, enzyme activities, and hemoglobin analysis and functional properties (e.g., Giarratana et al., Ibid.). The phenotype of cultured hematopoietic stem cells is assessed using microscopy of cells stained, for example, with Cresyl Brilliant blue. Reticulocytes exhibit a reticular network of ribosomal RNA under these staining conditions whereas erythrocytes are devoid of staining. Enucleated cells may also be monitored for standard hematological variables including mean corpuscular volume (MCV; fl), mean corpuscular hemoglobin concentration (MCHC; %) and mean corpuscular hemoglobin (MCH; pg/cell) using, for example, an XE2100 automat (Sysmex, Roche Diagnostics, Indianapolis, Ind.).

For the deformability measurements, for example, presumptive reticulocytes are separated from nucleated cells on day 15 of culture by passage through a de-leukocyting filter (e.g., Leucolab LCG2, Macopharma) and subsequently assayed using ektacytometry (instrument and protocols available from Bayer Corp., Tarrytown, N.Y.). The enucleated cells are suspended in 4% polyvinylpyrrolidone solution and then exposed to an increasing osmotic gradient from 60 to 450 mosM. Changes in the laser diffraction pattern (deformability index) of the cells are recorded as a function of osmolarity, to assess the dynamic deformability of the cell membrane. The maximum deformability index achieved at a physiologically relevant osmolarity is related to the mean surface area of red blood cells.

Alternatively, assays of hemoglobin may be used to assess the phenotype of differentiated cells (Giarratana et al., Ibid.). For example, high performance liquid chromatography (HPLC) using a Bio-Rad Variant II Hb analyzer (Bio-Rad Laboratories) is used to assess the percentage of various hemoglobin fractions. Oxygen equilibrium is measured using a continuous method with a double-wavelength spectrophotometer (e.g., a Hemox analyzer available from TCS, Medical Products Divsn., Southampton, Pa.). The binding properties of hemoglobin are assessed using flash photolysis. In this method, the rebinding of CO to intracellular hemoglobin tetramers is analyzed at 436 nm after photolysis with a 10 nanosecond pulse at 532 nm.

Red blood cells with HLA-DR1, NP (404-415) eptitope, and B7.1 on the cell surface are characterized and used for stimulating CD4+ T cell responses to influenza virus in vitro or in vivo. After trypsin cleavage of the DR1-B7.1 fusion protein, the HSC are tested for the expression of HLA-DR1 and B7.1 using fluorescent antibodies and a flow cytometer (see above for description of cytometry experiments). Artificial APC are sorted with a cytometer and used for in vitro or in vivo stimulation of CD4+ T cells.

Alternatively, to eliminate most of the intracellular components from aAPC, red cell ghosts are produced. Methods to produce red cell ghosts from red blood cells are known in the art (see e.g., Burgess et al., J. Physiol. 317: 67-90, 1981 which is incorporated herein by reference). Red blood cells are lysed by incubation in 1 mM CaCl₂, and 3 mM EGTA NaH₂PO₄ at 1° C. for approximately 3 minutes and resealed by the addition of KCl, to give an osmolarity of approximately 300 mosmol/L, and then incubated at 38° C. for 30 minutes. Prior to resealing, the red blood cell ghosts optionally take up at least one therapeutic agent (e.g., cytokine) for later release.

Artificial APC are used to stimulate anti-influenza immune responses from CD4+ T cells. Artificial APC are used in vitro with CD4+ T cells from a matched donor (i.e. same MHC Class II as aAPC) and antigen-specific proliferation is measured. A human T cell line, K68-36, specific for NP (404-415) presented in the context of HLA-DR1 is incubated with aAPC presenting the NP (404-415)-DR1-B7.1 fusion protein, and with control DR1 fusion proteins (e.g. DR1 with peptide from influenza hemaglutinin (HA). Proliferation assays are incubated 3-5 days at 37° C. in 5% CO₂ and pulsed with ³H thymidine for 18 hours. ³H thymidine incorporation into DNA is taken as a measure of proliferation (see U.S. Pat. No. 7,141,656 Ibid.) An aAPC with single chain HLA-DR1 joined with an influenza hemaglutinin (HA) epitope, HA (307-318) and B7.1 is used as a negative control.

### Prophetic Example 6

### Construction of Artificial Antigen Presenting Cells with Multiple Viral Epitopes

An artificial antigen presenting cell (aAPC) is constructed from a MHC class II (MHCII) protein joined with an antigenic peptide (e.g., epitope) and a co-stimulatory molecule, B7.1. The MHCII-epitope-B7.1 complex is inserted in the lipid bilayer of a liposome.

An aAPC is constructed from a liposome including a lipid bilayer with an embedded MHCII protein, HLA-DR1, that includes peptide epitopes: influenza nucleoprotein, amino acids 404-415 (NP 404-415), or influenza hemaglutinin, amino acids 307-318 (HA 307-318). Methods of making single chain MHC II proteins joined to antigenic peptides are known in the art (see e.g., U.S. Pat. No. 7,141,656, which is incorporated herein by reference). Complementary DNA (cDNA) for the HLA-DR1 α chain and HLA-DR1 β chain are obtained by molecular cloning using messenger RNA (mRNA) isolated from BLCL-K68 cells (a HLA-DR1 homozygous cell line), or mRNA from peripheral blood leukocytes of a HLA-DR1 positive individual. Methods to isolate mRNA, clone cDNA and determine DNA sequences are known (see e.g., U.S. Pat. No. 7,141,656, Ibid. and Sambrook and Russell, “Molecular Cloning: A Laboratory Manual”, (Third Edition, 2001, Cold Spring Harbor Laboratory Press, Woodbury, N.Y.), each of which is incorporated herein by reference). An oligonucleotide (available from Sigma-Aldrich Chem. Co., St. Louis, Mo.) encoding the influenza peptide NP (404-415) or HA (307-318) is joined with the 5′ end of separate cDNA segments encoding the HLA-DR1 β chain (see e.g., U.S. Pat. No. 7,141,656, Ibid.) The NP(404-415)-DR1 β chain gene or the HA(307-318)-DR1β chain gene, and a gene for DR1 α chain are inserted in a bicistronic mammalian cell expression vector with an internal ribosome entry site (IRES) (see e.g., Product Information Sheet: “pIRES Vector” available from Clontech Laboratories, Inc., Mountain View, Calif., the subject matter of which is incorporated herein by reference). A DNA sequence encoding a Myc tag is fused to the 3′ end of the DR1 α chain, thus encoding a Myc epitope adjacent to the carboxyl terminal cytoplasmic domain of the DR1 α chain. The NP (404-415)-DR1β-DR1α-Myc construct is shown in Figure A below. DNA sequences and a monoclonal antibody for Myc are known in the art (see e.g., the Product Information Sheet: “Myc and HA Tagged Mammalian Expression Vectors” available from Clontech Laboratories, Inc., Mountain View, Calif. which is incorporated herein by reference). A second bicistronic mammalian cell expression vector encoding the costimulatory molecule B7.1 (also known as CD80) and a bispecific antibody is constructed with an alternate selectable marker, e.g. dihydrofolate reductase (DHFR), to allow co-selection of the B7.1 vector and the HLA-DR1 vector with methotrexate and G418, respectively. A hemaglutinin (HA) tag sequence is added to the carboxyl terminus of B7.1 by fusing DNA sequences for B7.1 and HA “in frame” to encode a B7.1-HA fusion protein. See Figure B.

The bicistronic expression vector encoding B7.1-HA also contains a cistron for a single chain bispecific antibody (scBisp Ab) that recognizes Myc and HA. HLA-DR1 and B7.1 proteins are joined in the cytoplasm with a bispecific antibody (Bisp Ab) that recognizes the cytoplasmic tags on each membrane protein. An intracellular Bisp Ab recognizing Myc and HA is encoded in the bicistronic vector (see Figure B). Methods to make intracellular antibodies that function inside the cell are known in the art (see e.g., U.S. Patent App. Pub. No. 2010/0042072 and Visintin et al., P.N.A.S. USA 96: 11723-11728 (1999), each of which is incorporated herein by reference). Methods to make Bisp Abs are known in the art (see e.g., Herrmann et al., Cancer Res. 68: 1221-1227 (2008), which is incorporated herein by reference). A bispecific single chain antibody fragment with multiple variable region domains is derived from an intracellular anti-Myc antibody and an intracellular anti-HA antibody. Methods to select antibodies from phage display single chain variable fragment (scFv) libraries are known in the art (see e.g., Pansri et al., BMC Biotech. 9(6): 2009, doi: 10.1186/1472-6750-9-6 and Visintin et al. Ibid., which are incorporated herein by reference). A DNA construct encoding a single chain Bisp Ab construct with tandem scFv regions is inserted into the mammalian cell expression vector encoding B7.1. See Figure B. The NP (404-415)-DR1-Myc vector, the HA (307-318)-DR1-Myc vector and the B7.1-HA-scBisp Ab vector are co-transfected into Chinese hamster ovary (CHO) cells (available from American Type Culture Collection, Manassas, Va.). Production and purification of the Bisp Ab are well known in the art (see e.g., Herrman, Ibid.) HLA-DR1-Myc and B7.1-HA proteins are joined by the Bisp Ab that binds both proteins via their cytoplasmic tags, Myc and HA. See Figure C. Protein fractions containing HLA-DR1 and B7.1 proteins joined by a Bisp Ab are purified and incorporated into liposomes to create artificial antigen presenting cells.

Methods for molecular cloning and co-expression of MHC II and co-stimulatory genes are known in the art (see e.g., U.S. Pat. No. 7,439,335, which is incorporated herein by reference). Chinese hamster ovary (CHO) cells are co-transfected with the NP (404-415)-DR1-Myc vector or HA (307-318)-DR1-Myc vector and the B7.1-HA-scBisp Ab vector using Lipofectamine™ (available from Life Technologies, Carlsbad, Calif.) and stable clones are selected for resistance to both G418 and methotrexate. To test for the expression of both proteins, the cells are stained with fluorescent antibodies and analyzed on a flow cytometer (antibodies, reagents, protocols and flow cytometers are all available from BD Biosciences, San Jose, Calif.). Stable CHO cell lines expressing both B7.1 and HLA-DR1 (with the joined epitope NP (404-415) or HA (307-318)) are expanded in a bioreactor. The proteins are isolated using a Mem-PER Eukaryotic Membrane Protein Extraction Kit available from Thermo Fisher Scientific (Rockford, Ill.). As described herein, proteins are purified by affinity chromatography, or other means known in the art. Alternatively, in certain aspects, lipid rafts are utilized for protein isolation and purification.

Liposomes are prepared from cholesterol and L-α-phosphatidylcholine using methods known in the art (see e.g., U.S. Patent Application No. 2005/0208120, which is incorporated herein by reference). Cholesterol and L-α-phosphatidyl choline are combined at a molar ratio of 2:7 in chloroform and the chloroform is evaporated away using an argon stream. The liposomes are resuspended in a 140 mM NaCl, 10 mM Tris HCI, 0.5% deoxycholate at pH 8 and sonicated for three minutes. HLA-DR1 and B7.1 joined by a Bisp Ab are inserted into the liposomes by combining the HLA-DR1:B7.1 complexes with liposomes at a 1:10 molar ratio and dialyzing for 72 hours at 4° C. versus phosphate buffered saline. The liposomes are characterized to assess liposome size and the amount of HLA-DR1 and B7.1 protein incorporated into the liposomes. Liposome size is determined using dynamic light scattering and flow cytometry (see e.g., U.S. Patent Application No. 2005/0208120, Ibid.). For example, liposomes containing HLA-DR have a mean diameter of approximately 50 nanometers. To measure HLA-DR1 and B7.1 protein on the liposomes the liposomes are analyzed on a flow cytometer after staining with FITC labeled anti-DR antibody. Liposomes are sorted based on FITC fluorescence, forward scatter and side scatter to isolate and count liposomes with HLA-DR1. HLA-DR and B7.1 protein on the liposomes is measured using an enzyme-linked immunosorbent assay (ELISA). Methods to analyze liposomes by flow cytometry and to quantitate HLA-DR and other proteins by ELISA are known in the art (see e.g., U.S. Patent Application No. 2005/0208120, Ibid.).

### Prophetic Example 7

### Method for Vaccinating an Individual with an Antigen Presenting Cell Displaying Multiple Epitopes of a Virus

The elderly, young children, and other individuals at increased risk potential from influenza viral infection are vaccinated with an artificial APC (aAPC) comprised of fused vesicles containing selected major histocompatibility complex II (MHC II) proteins joined to multiple influenza epitopes that will elicit immunity to a number of influenza subtypes arising from antigenic drift. The aAPC contain fusion proteins that join influenza eptitopes, HLA-DR, B7.1 (CD80), and peptide sequences from PAR2 that contain a trypsin cleavage site. The aAPC are constructed with multiple fusion proteins containing different epitopes derived from the hemaglutinin (HA) proteins of different influenza subtypes. The aAPC are used for in vitro and in vivo immunization to elicit T cell and B cell immunity to a number of influenza subtypes.

Artificial APC are administered to children and the elderly to protect them from influenza A by eliciting T cell and B cell immunity to multiple influenza A virus subtypes. The aAPC protects against influenza A viruses that undergo recombination and emerge as different viral subtypes with different viral antigens. The aAPC is constructed from HLA-DR proteins that match the individual patient's MHC, and contains epitopes derived from the HA proteins found in different influenza subtypes (e.g., H1N1, H2N2, H3N2, or H5N1, where H denotes the hemaglutinin variant and N denotes the neuraminidase variant). To identify each patient's HLA-DR alleles, their DNA is genotyped at high resolution by using a combination of oligonucleotide sequence specific amplification and DNA sequencing; this will determine the identity of the 2 HLA-DR β-chain genes at the maternal and paternal HLA-DRBI loci. Methods to determine HLA genotypes and HLA antigen expression are known in the art (see e.g., Nowak, Bone Marrow Transplant. 42: s71-s76, 2008 which is incorporated herein by reference). For example, a patient may have HLA-DRB1*0101 at one locus and HLA-DRB1*0301 at the other locus. Once the patient's HLA-DR alleles are known, T cell epitopes from influenza HA antigens can be identified. HA peptide sequences that are bound by specific HLA-DR alleles and known to elicit CD4+ T cell responses are known in the art (see e.g., Bui, et al., Proc. Natl. Acad. Sci. USA 104: 246-251, 2007, which is incorporated herein by reference). For example, immunogenic HA epitopes from influenza subtypes H1N1, H3N2, and H5N1 that are restricted by HLA-DRB1*0101 and HLA-DRB1*0301 are known. The HA peptide PKYVKQNTLKLAT (amino acids number 322-334) from subtype H3N2, which is presented by DRB1*0101 and DRB1*0301, and other HA peptides derived from viral subtypes H1N1 and H5N1 that are presented by specific HLA-DR alleles, are selected from the Immune Epitope Database and Analysis Resource (IEDB; see e.g., Bul et al., Ibid.).

Artificial APC are constructed with membrane bound fusion proteins expressed using mammalian cell expression vectors. For example, a fusion protein encodes the influenza subtype H3N2 HA epitope HA (322-334), a HLA-DR protein (e.g., genes denoted DRB1*0101 and DRA), the co-stimulatory molecule B7.1, and sequences from PAR2, including a cytoplasmic loop, transmembrane domain (TMD), and an exodomain with a protease cleavage site. Methods to make single chain MHC II proteins joined to antigenic peptides are known in the art (see e.g., Zhu et al., Eur. J. Immunol. 27: 1933-1941, 1997; and U.S. Pat. No. 7,141,656, each of which is incorporated herein by reference). Complementary DNA (cDNA) for the HLA-DR α chain and HLA-DR β chain are obtained by molecular cloning using messenger RNA (mRNA) from human lymphoblastoid cells (a HLA-DRB1*0101 homozygous cell line). Methods to isolate mRNA, clone cDNA and determine DNA sequences are known in the art (see e.g., U.S. Pat. No. 7,141,656, Ibid. and Sambrook and Russell, “Molecular Cloning: A Laboratory Manual”, (Third Edition, 2001, Cold Spring Harbor Laboratory Press, Woodbury, N.Y.), each of which is incorporated herein by reference). An oligonucleotide (available from Sigma-Aldrich Chem. Co., St. Louis, Mo.) encoding the influenza HA peptide HA (322-334) is joined with the 5′ end of a cDNA segment encoding the HLA-DR1 β chain (see e.g., U.S. Pat. No. 7,141,656, Ibid.). A construct encoding a single chain HLA-DR1 (scDR1) is constructed with the HA (322-334) epitope-DR1 β chain (extracellular domain) and DR α chain joined (see e.g., Zhu et al., Ibid.). The scDR1 construct is joined to DNA sequences derived from PAR2 and B7.1. The cytoplasmic domain of the scDR1 (derived from the carboxy terminus of the HLA-DR1 α chain) is joined to the first cytoplasmic loop (amino acids (a.a.) 101-107), the second TMD (a.a. 108-128), and part of the exodomain (a.a. 28-40) of PAR2 including a trypsin cleavage site (see Figures A and B). Methods and compositions to make PAR2 fusion proteins are known in the art (see e.g., Nystedt et al., Eur. J. Biochem. 232: 84-89, 1995 and Bae et al., J. Thromb. Haemost. 6: 954-961, 2008, which are incorporated herein by reference). The mature amino terminus of the B7.1 sequence is joined adjacent to the PAR2 trypsin cleavage site. A diagram of the encoded fusion protein (Figure A) displays the fusion protein starting at the amino terminus and encompassing HA (322-334), scDR1, PAR2 and B7.1 at the carboxyl terminus. The DNA construct encodes the scDR1-B7.1 fusion protein and is inserted into a lentivirus vector, and expressed in Chinese hamster ovary (CHO) cells. Lentiviral vectors and methods for gene expression are known in the art (see e.g., “Lenti-X™ Lentiviral Expression Systems User Manual” available from Clonetech Laboratories, Inc., Mountain View, Calif., the subject matter of which is incorporated herein by reference). DNA sequences encoding the scDR1-B7.1 fusion protein are cloned into a plasmid-based expression vector containing required elements for packaging the expression construct into virions. The plasmid is combined with a packaging mixture and transfected into a 293T cell line (available from American Type Culture Collection, Manassas, Va.) to produce a recombinant, non-replicating lentivirus. Lentiviral stocks with a titer of approximately 10⁵ to 10⁷ transducing units/ml are sufficient to transduce 10⁶-10⁸ CHO cells at a multiplicity of infection of 1.0. To determine the titer of the lentivirus stock, serial ten-fold dilutions of the stock are applied to a HT-1080 cell line (available from American Type Culture Collection, Manassas, Va.), and the number of transduced cells is counted after growth in puromycin since a puromycin resistance gene is incorporated in the lentiviral expression vector to allow selection of stably transduced cells. CHO cells are transduced with the lentiviral vector encoding the scDR1-B7.1 fusion protein to create stable cell lines that express joined HA (322-334)-scDR1 and B7.1 on their plasma membrane. See Figure B.

CHO cells transduced with the lentivirus encoding joined HA epitope, scDR1, and B7.1 are treated with trypsin in vitro to cleave the trypsin site adjacent to B7.1 in the fusion protein (see Fig. B). The trypsin cleavage site in PAR2 is cleaved by approximately 1 nM trypsin in vitro (see e.g., Nystedt et al., Ibid.), and since the normal range for human serum trypsin concentration is between 5.7-16.4 nM (Artigas et al., Postgrad. Med. J. 57: 219-222 (1981), which is incorporated herein by reference), it will also be cleaved in vivo. After trypsin cleavage of the scDR1-B7.1 fusion protein, the CHO cells are tested for the expression of HLA-DR1 and B7.1. The cells are stained with fluorescent antibodies and analyzed on a flow cytometer (antibodies, reagents, protocols and flow cytometers are all available from BD Biosciences, San Jose, Calif.). Cells displaying DR1 and B7.1 on the cell surface are sorted based on immunofluorescence with fluor-conjugated antibodies to select a stable CHO cell line expressing the fusion protein. The CHO cell line is expanded in a bioreactor to provide a source of HLA-DRB1*0101/-DRA joined with a H3N2 HA epitope and B7.1. The proteins are isolated using a Mem-PER Eukaryotic Membrane Protein Extraction Kit available from Thermo Fisher Scientific (Rockford, Ill.). As described herein, the proteins are purified by affinity chromatography, or other means known in the art. Alternatively, in certain aspects, lipid rafts are utilized to isolate and purify proteins of an embodiment.

Liposomes are prepared from cholesterol and L-α-phosphatidylcholine using methods known in the art (see e.g., U.S. Patent Application No. 2005/0208120, which is incorporated herein by reference). Cholesterol and L-α-phosphatidyl choline are combined at a molar ratio of 2:7 in chloroform. The chloroform is evaporated away under nitrogen. Next, the liposomes are resuspended in a 140 mM NaCI, 10 mM Tris HCI, 0.5% deoxycholate at pH 8, and sonicated for three minutes. HA (322-334)-scDR1-B7.1 fusion proteins are inserted in the liposomes by combining the protein complex with liposomes at a 1:10 molar ratio and dialyzing for 72 hours at 4° C. versus phosphate buffered saline. The liposomes are characterized to assess liposome size and the amount of HLA-DR1 and B7.1 protein incorporated in the liposomes. Liposome size is determined using dynamic light scattering and flow cytometry (see e.g., U.S. Patent Application No. 2005/0208120, Ibid.). For example, liposomes containing HLA-DR are generally expected to have a mean diameter of approximately 50 nanometers. To measure HLA-DR1 and B7.1 protein on the liposomes, the aAPCs are analyzed on a flow cytometer after staining with FITC labeled anti-DR antibody. Liposomes are sorted based on FITC fluorescence, forward scatter and side scatter to isolate and count the liposomes with HLA-DR1. HLA-DR and B7.1 protein on the liposomes is measured using an enzyme-linked immunosorbent assay (ELISA). Methods to analyze liposomes by flow cytometry and to quantitate HLA-DR and other proteins by ELISA are known in the art (see e.g., U.S. Patent Application No. 2005/0208120, Ibid.). Liposomes may be constructed with multiple epitope-HLA-DR-B7.1 fusion proteins. For example, combining liposomes with fusion proteins encompassing variant epitopes derived from different influenza subtypes (e.g. H1N1, H3N2, and H5N1) creates aAPC immunogenic for multiple viral subtypes.

Individuals at increased potential risk from influenza infection (i.e., children and the elderly) are immunized with aAPC containing HLA-DR alleles matching their genotype and containing HA epitopes representative of multiple subtypes of influenza virus. Approximately 2×10⁸ aAPC are administered intravenously to elicit CD4⁺ T cell immunity versus seasonal and/or pandemic subtypes of influenza.

### Equivalents

The present disclosure is not to be limited in terms of the particular embodiments described in this application, which are intended as single illustrations of individual aspects of. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

For any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. All language such as “up to,” “at least,” “greater than,” “less than,” and the like include the number recited and refer to ranges which can be subsequently broken down into subranges as discussed above. Finally, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The various aspects and embodiments disclosed herein are for purposes of illustration and are not intended to be limiting, with the true scope and spirit being indicated by the following claims.

The various aspects and embodiments disclosed herein are for purposes of illustration and are not intended to be limiting, with the true scope and spirit being indicated by the following claims.

All publications and patent applications cited in this specification are herein incorporated by reference to the extent not inconsistent with the description herein and for all purposes as if each individual publication or patent application were specifically and individually indicated to be incorporated by reference for all purposes.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A composition, comprising:
a lipid surface including at least one actively controllable artificial antigen presenting cell complex, the at least one actively controllable antigen presenting cell complex including at least one epitope joined to at least one MHC receptor component.

2. (canceled)

3. The composition of claim 1, wherein the at least one actively controllable artificial antigen presenting cell complex includes at least one switchable complex.

4. The composition of claim 1, further comprising at least one immunomodulatory molecule component joined to the at least one MHC receptor component.

5. The composition of claim 4, wherein the at least one immunomodulatory molecule component joined to the at least one MHC receptor component is joined by at least one linker or linking component.

6. The composition of claim 4, wherein the at least one linker includes at least one cleavable linker.

7. The composition of claim 6, wherein the at least one linker includes at least one of a chemically cleavable linker, optically cleavable, thermally cleavable linker, optically cleavable linker, or enzymatically cleavable linker.

8. The composition of claim 7, wherein the at least one cleavable linker includes at least one of an intracellular linker, extracellular linker, or a linker embedded in the lipid surface.

9. The composition of claim 4, wherein two or more different immunomodulatory components are bound to the at least one MHC receptor component.

10-12. (canceled)

13. The composition of claim 4, wherein the at least one immunomodulatory molecule component includes at least a portion of one or more of a co-stimulatory molecule, accessory molecule, adhesion molecule, cytokine, cytokine receptor, chemokine, chemokine receptor, anergy-inducing molecule, apoptosis-inducing molecule, or differentiation-inducing molecule.

14-15. (canceled)

16. The composition of claim 1, wherein at least a portion of the at least one actively controllable artificial antigen presenting cell complex is anchored to the lipid surface.

17. The composition of claim 1, wherein at least a portion of the at least one actively controllable artificial antigen presenting cell complex is embedded within the lipid surface.

18. The composition of claim 1, wherein at least a portion of the at least one actively controllable artificial antigen presenting cell complex traverses the lipid surface.

19-21. (canceled)

22. The composition of claim 1, wherein the lipid surface includes at least a portion of at least one of a liposome, lipid droplet, chemical emulsion, phase separation, exosome, micelle, platelet, chip, device, cell, cerasome, lipid monolayer, lipid bilayer, lipid multilayer, or red blood cell ghost.

23. (canceled)

24. The composition of claim 22, wherein the cell further includes at least one death-initiating component.

25. The composition of claim 24, wherein the at least one cell death-initiating component includes at least one cell death-initiating nucleic acid construct.

26. The composition of claim 25, wherein the at least one cell death-initiating nucleic acid construct includes at least one inducible regulatory element.

27. The composition of claim 25, wherein the at least one cell death-initiating nucleic acid construct encodes at least one gene product sufficient to initiate death of the modified cell.

28. The composition of claim 25, wherein the at least one cell death-initiating nucleic acid construct is configured to initiate programmed cell death of the cell.

29. The composition of claim 25, wherein the at least one cell death-initiating nucleic acid construct is configured to initiate at least one of necrosis, pyroptosis, autophagocytosis, or apoptosis of the cell.

30. The composition of claim 25, wherein the at least one cell death-initiating nucleic acid construct encodes at least one programmed cell death gene product.

31. (canceled)

32. (canceled)

33. The composition of claim 25, wherein the at least one death-initiating nucleic acid construct includes at least One gene product configured to lyse the at least one cell.

34. The composition of claim 33, wherein the at least one gene product to lyse the at least one cell includes at least one of a nuclease gene, or lysis gene E.

35. The composition of claim 25, wherein the at least one death-initiating nucleic acid construct encodes at least one gene product configured to interfere with the utilization of at least one cellular metabolite.

36. The composition of claim 25, wherein the at least one cell death-initiating component includes at least one receptor configured to allow entry of at least one of a toxin or pathogen.

37. The composition of claim 25, wherein the at least one cell death-initiating component includes at least one energy absorbing structure.

38. (canceled)

39. The composition of claim 1, wherein the MHC receptor component includes at least a portion of one or more of a Major Histocompatibility Class I protein, Major Histocompatibility Class II protein, or Major Histocompatibility Class III protein.

40. The composition of claim 1, wherein the at least one MHC receptor component includes at least a portion of one or more of β-2 microglobulin, Transporter Associated with Antigen Processing (TAP), MHC class I α-1 domain, MHC class I α-2 domain, MHC class I α-3 domain, tapasin, calreticulum, ERP57, or calnexin.

41. The composition of claim 1, wherein the at least one MHC receptor component includes at least one of human leukocyte antigen (HLA), H-Y, H-2, dog leukocyte antigen (DLA), bovine leukocyte antigen (BOLA), equine leukocyte antigen (ELA), swine leukocyte antigen (SLA), Rhesus monkey leukocyte antigen (RhL-A), B-locus (chicken), feline leukocyte antigen (FLA), or chimpanzee leukocyte antigen (ChL-A).

42. The composition of claim 1, wherein the at least one MHC receptor component includes at least a portion of one or more of a MHC class II α domain, or a β domain.

43. The composition of claim 42, wherein the at least a portion of the MHC class II α domain includes at least a portion of one or more of α-1 domain or α-2 domain.

44. The composition of claim 42, wherein the at least a portion of the β domain includes at least a portion one of one or more of β-1 domain or β-2 domain.

45. The composition of claim 1, wherein the at least one MHC receptor component encodes at least one gene product of one or more of HLA-A, HLA-B, HLA-C, HLA-DPA1, HLA-DPB1, HLA-DRA, HLA-DRB1, HLA-DQA1, or HLA-DQB1 genes.

46. The composition of claim 1, further comprising at least one therapeutic agent.

47. The composition of claim 46, wherein the at least one therapeutic agent is included in one or more of internal to the lipid surface, embedded in the lipid surface, or transversing the lipid surface.

48-52. (canceled)

53. The composition of claim 46, wherein the at least one therapeutic agent includes at least one vaccine.

54. The composition of claim 53, wherein the at least one vaccine includes at least one of an antigenic peptide, antigenic protein, or antigenic carbohydrate.

55. The composition of claim 53, wherein the at least one vaccine includes at least one of an envelope protein, capsid protein, surface protein, toxin, polysaccharide, or oligosaccharide.

56. The composition of claim 53, further comprising at least one adjuvant.

57. The composition of claim 46, wherein the at least one therapeutic agent includes at least one cytokine.

58-72. (canceled)

73. The composition of claim 1, further comprising at least one nanoparticle.

74. The composition of claim 73, wherein the at least one nanoparticle includes at least one taggant, contrast agent, sensor, semiconductor, or electronic identification device.

75. The composition of claim 74, wherein the at least one electronic identification device includes at least one radio frequency identification device (RFID).

76. The composition of claim 73, wherein the at least one nanoparticle includes at least one of a diamagnetic particle, ferromagnetic particle, paramagnetic particle, super paramagnetic particle, particle with altered isotope, or other magnetic particle.

77. The composition of claim 73, wherein the at least one nanoparticle includes at least one quantum dot, silica nanoparticle, nanotube, or x-ray absorber.

78. The composition of claim 73, wherein the at least one nanoparticle includes at least one heavy metal.

79. The composition of claim 1, further comprising at least one radioactive, luminescent, colorimetric, or odorous substance.

80. The composition of claim 1, further comprising at least one photoactivatable molecule.

81. The composition of claim 80, wherein the at least one photoactivatable molecule includes psoralen.

82-83. (canceled)

84. The composition of claim 1, wherein the at least one antigen presenting cell complex includes at least two different epitopes joined to the at least one MHC receptor component.

85-89. (canceled)

90. The composition of claim 1, wherein at least two of the components of the antigen presenting cell complex are displayed in a pre-determined arrangement.

91. The composition of claim 90, wherein the at least one pre-determined arrangement is determinable by one or more desired immune responses.

92. The composition of claim 90, wherein the at least one pre-determined arrangement includes a pre-determined spatial arrangement.

93. The composition of claim 90, wherein the at least one pre-determined arrangement includes a pre-determined temporal sequence.
